(19) United States
(12) Patent Application Publication
(10) Pub. No.: US 2024/0342414 A1
(43) Pub. Date: Oct. 17, 2024

(54) PATIENT INTERFACE WITH ADHESIVE SURFACE

(71) Applicant: ResMed Pty Ltd, Bella Vista, New South Wales (AU)

(72) Inventors: Lachlan Richard GOLDSPINK, Sydney (AU); Michael Christopher HOGG, Sydney (AU); Stewart Joseph WAGNER, Sydney (AU); Vinay MANJUNATH, Sydney (AU); Bruce Richard DAVIES, Sydney (AU)

(21) Appl. No.: 18/682,417

(22) PCT Filed: Aug. 9, 2022
(86) PCT No.: PCT/AU2022/050865

§ 371 Date: Feb. 8, 2024

(30) Foreign Application Priority Data
Aug. 9, 2021 (AU) 2021902463
Apr. 11, 2022 (AU) 2022900954

(51) Int. Cl. A61M 16/06

(52) U.S. Cl. CPC A61M 16/0605; A61M 16/0688; A61M 2205/0216; A61M 2205/3331; A61M 2209/088; A61M 2210/0618

(57) ABSTRACT

A patient interface comprising a seal-forming structure with one or more adhesive surfaces is provided. The seal-forming structure is configured to form a seal with a region of the patient's face surrounding an entrance to the patient's airways. The patient interface may further comprise a plenum chamber pressurisable to a therapeutic pressure of at least 6 cmH₂O above ambient air pressure. A flow of breathable gas at the therapeutic pressure is provided through an air circuit. The seal-forming structure may be configured to adhere to inferiorly facing surfaces of the patient's nose, e.g. the alar rim region. In certain forms the patient interface comprises a decoupling structure configured to at least partly de-couple the seal-forming structure from the air circuit

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use. The present technology relates specifically to seal-forming structures for patient interfaces which form a seal with a patient's airways through adhesive surfaces.

### 1.2 DESCRIPTION OF THE RELATED ART

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

A range of therapies have been used to treat or ameliorate such conditions. Furthermore, otherwise healthy individuals may take advantage of such therapies to prevent respiratory disorders from arising. However, these have a number of shortcomings.

One of the major issues in respiratory therapy is adherence, which is also referred to as compliance. Usually, a patient may be required to don a patient interface for prolonged periods as part of the respiratory therapy. Bulky and/or obtrusive patient interfaces often lead to patients discontinuing the respiratory therapy due to discomfort, inconvenience or interference with sleep. In particular, it is difficult to ensure that infants and children do not remove patient interface during respiratory therapy.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and/or data management.

### 1.2.3.1 Patient Interface

A patient interface may be used to interface respiratory equipment to its wearer, for example by providing a flow of air to an entrance to the airways. The flow of air may be provided to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure.

Conventionally, mask systems are used as patient interfaces to convey the flow of air. These mask systems typically include a plenum chamber which is secured against the patient's face through headgear. The plenum chamber, with the patient's face, encloses a volume of space and protrudes away from the patient's face to accommodate the facial features of the patient such as their nose and/or mouth. Often, the plenum chamber may be made of a rigid material. These aspects of the design of some conventional patient interfaces can make sleeping while wearing the patient interface on inconvenient, uncomfortable and potentially claustrophobic for the patient.

Mask systems other than those typically used for respiratory therapy may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. As mentioned earlier, this discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy.

It is often recommended that a patient regularly wash their mask, if a mask is required to be cleaned, or if it is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and/or a nasal bridge region of a face. These different types of patient interfaces may be known by a variety of names by their manufacturer including nasal cushions, nasal pillows, and nasal puffs.

In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These patient interfaces may be referred in the art as oral cushions, oro-nasal cushions or full face cushions.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a molded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. A seal formed by an adhesive is usually highly effective with little or no leak for typical therapy pressures (e.g. up to 20 cmH₂O).

Regular application and removal of an adhesive-based seal-forming structure may cause skin trauma or irritation. Moreover, conventional adhesive-based seal-forming structures require cleaning of the area of the skin to which the seal-forming structure is to be adhered before affixing the seal-forming structure. Repeated cleaning, which may include alcohol swabbing, can cause damage to the skin.

Further, affixing an adhesive-based seal-forming structure in or around the nasal region may lead to weakening of adhesion due to moisture from the patient's breath. In other regions of the face, the skin may also release moisture which can loosen adhesion, thereby causing leakages which can lead to ineffective respiratory therapy. In addition, when a patient is subjected to oxygen therapy, leakages can lead to unnecessary loss of the oxygen gas. This leakage of oxygen may be particularly disadvantageous in developed countries where medical oxygen is a scarce and expensive resource.

Adhesive-based seal-forming structures may also leave a residue, odour or colour on the patient's skin, sometimes even after the seal-forming structure is removed.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 1.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. One advantage of the use of adhesives to position and stabilise the seal-forming structure on the patient's face is that it avoids the need for headgear (discussed below), which can be uncomfortable, claustrophobic and adds manufacturing cost and complexity. However, as mentioned before, the use of adhesives, as is known in the art, has some disadvantages.

Another technique is the use of one or more straps and/or stabilising harnesses. Many such harnesses suffer from being one or more of ill-fitting, bulky, uncomfortable and awkward to use. They tend to be less air-tight than adhesive-based seal forming structures. Moreover, straps and/or stabilising harnesses tend to leave markings on the face when used overnight.

### 1.2.3.1.3 Pressurised Air Conduit

In one type of treatment system, a flow of pressurised air is provided to a patient interface through a conduit in an air circuit that fluidly connects to the patient interface so that, when the patient interface is positioned on the patient's face during use, the conduit extends out of the patient interface forwards away from the patient's face. This may sometimes be referred to as a “tube down” configuration.

Conduits connecting to an interface at the front of a patient's face may sometimes be vulnerable to becoming tangled up in bed clothes.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus, RPT devices may also act as flow therapy devices. Examples of RPT devices include a CPAP device and a ventilator.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 1.2.3.5 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

An aspect of certain forms of the present technology is to provide methods and/or apparatus that improve the compliance of patients with respiratory therapy.

An aspect of one form of the present technology is a patient interface comprising a seal-forming structure which is configured to form a seal with a region of the patient's face surrounding an entrance to the patient's airways.

Another aspect of one form of the present technology is a patient interface comprising a seal-forming structure having an opening such that a flow of breathable gas is delivered to at least an entrance to the patient's nares.

Another aspect of one form of the present technology is a patient interface comprising a seal-forming structure which further comprises at least one adhesive surface configured in use to adhere to the region of the patient's face surrounding the entrance to the patient's airways to form the seal.

An aspect of one form of the present technology is a patient interface comprising a plenum chamber pressurisable to a therapeutic pressure of at least 6 cmH₂O above ambient air pressure, said plenum chamber including a plenum chamber inlet port configured to receive a flow of breathable gas at the therapeutic pressure for breathing by the patient.

In one form of the present technology, the seal-forming structure is configured to maintain said therapeutic pressure in the plenum chamber throughout the patient's respiratory cycle in use.

In one form, the seal-forming structure may comprise an opening through which breathable gas is conveyed to the patient's airways.

Another aspect of one form of the present technology is a patient interface that has a perimeter shape which is complementary to that of an intended wearer.

In one form, the seal-forming structure is configured to have a perimeter shape which is complementary to the region of the patient's face surrounding the entrance to the patient's airways to form the seal.

The regions to which the seal-forming structure is to be adhered to may be referred to as the target sealing regions.

In one form, the seal-forming structure is configured such that the region of the patient's face comprises regions of the patient's face adjacent to the nares.

In one form, the seal-forming structure is configured such that the region of the patient's face comprises inferiorly facing surfaces of the nose and a superior region of the lip superior. For example, the region may comprise inferiorly facing surfaces of the patient's nasal alar. For example, the region may comprise a superior-most region of the patient's lip superior. In one form, the region of the patient's face may comprise an anterior region of the nose that is immediately inferior to the patient's pronasale.

In one form, the seal-forming structure is configured such that the region of the patient's face comprises side regions of the nasal alar, for example inferior regions of the side regions of the nasal alar.

In one form, the seal-forming structure is configured such that, when the seal-forming structure is adhered to the patient's face, a superior-most portion of the seal-forming structure adheres to the patient's alar crease.

In certain forms, the seal-forming structure may be configured such that the region of the patient's face comprises regions between the patient's nasal alar and the patient's nasolabial sulcus.

In certain forms, the seal-forming structure is configured so that the shape of the at least one adhesive surface substantially resembles the region of the patient's face.

An aspect of one form of the present technology is a patient interface comprising a seal-forming structure which may be a substantially D-shaped band.

In one form, the D-shaped form may comprise one or more of a stem portion, two corner portions, two shoulder portions and an apex portion. In one form, the corner portions may be located at either end of the stem portion. In another form, the shoulder portions may connect each corner portion to the apex portion. In one form, the seal-forming structure may have reflectional symmetry about an axis which is positioned on the patient's sagittal plane, and which passes through the apex portion and a mid-section of the stem portion. In one form, the stem portion may be substantially straight. In another form, the stem portion may be arced radially inwardly such that to form a concave recess in a mid-section of its radially outer side and a convex protrusion in a mid-section of its radially inner side. In one form, the recess and the protrusion may be generally rounded in shape. In one form, the corner portions may be generally triangular in shape and may each comprise an edge on the radially outer side on the same side of the seal-forming structure as the recess of the stem portion. In one form, the edge may be angled to create an obtuse corner between the angled edge and the recess of the stem portion. In one form, the apex portion may comprise a concave recess on its radially inner side and a convex protrusion on its radially inner side. The recess and the protrusion may be generally rounded in shape. In one form, the apex portion is configured to adhere to an anterior region of the nose that is inferior to the pronasale. In one form, the shoulder portions are configured to adhere to inferior regions of the nasal alar. In one form, the corner portions are configured to adhere to the alar crease region including, but not limited to, the alar crease and/or the laterally outer regions of the nasal alar and/or the regions of the cheek adjacent the alar crease. In one form, the stem portion is configured to adhere to a superior region of the lip superior.

In one form, the seal-forming structure comprises a notch in an edge of a region of the seal-forming structure configured to adhere to a region of the patient's nose substantially inferior to the patient's pronasale in use.

An aspect of one form of the present technology is a patient interface system comprising multiple seal-forming structures having a plurality of shapes and sizes.

In one form, the seal-forming structures are interchangeably connectable to the plenum chamber of the patient interface. Any one of the plurality of seal-forming structures may be able to be used as part of the patient interface such that a patient may be able to select the seal-forming structure that is shaped and sized to best suit their needs. For instance, the patient may choose a seal-forming structure whose shape corresponds closest to their facial features which lie in the regions of the patient's face to which the seal-forming structure is to be attached. Further, the patient may also choose a seal-forming structure that provides maximum comfort.

A patient interface system for use in delivering breathable gas to a patient. The patient interface system may comprise a plenum chamber pressurisable to a therapeutic pressure of at least 6 cmH₂O above ambient air pressure. The plenum chamber may comprise a plenum chamber inlet port configured to receive a flow of breathable gas at the therapeutic pressure for breathing by the patient. The patient interface system may further comprise a vent structure to allow a continuous flow of gases exhaled by the patient from an interior of the plenum chamber to ambient. The vent structure may be configured to maintain the therapeutic pressure in the plenum chamber in use. The patient interface system may further comprise a first seal-forming structure and a second seal-forming structure. The first and second seal-forming structure may be interchangeably connectable to the plenum chamber. The first seal-forming structure may have a different size and/or shape from the second seal-forming structure. Each of the first and second seal-forming structures may be configured to form a seal with a region of the patient's face surrounding an entrance to the patient's airways. Each of the first and second seal-forming structures may have an opening therein such that the flow of breathable gas is delivered to at least an entrance to the patient's nares. Each of the first and second seal-forming structures may be configured to maintain said therapeutic pressure in the plenum chamber throughout the patient's respiratory cycle in use. Each of the first and second seal-forming structures may comprise at least one adhesive surface configured in use to adhere to a region of the patient's face to form the seal.

In one form, the shapes of the seal-forming structures are configured so that at least one of the shapes is able to fit most patients in a given population of patients. This means that a patient interface is able to be configured to provide a good level of fit with only a discrete number of generic shapes and sizes of seal-forming structures. Producing only a discrete number of seal-forming structures may provide cost efficiencies in manufacturing and supply.

In an alternative form, a different number of different shapes and sizes of seal-forming structures may be provided. The greater the number of seal-forming structures provided, the more likely any individual patient is to be able to find a seal-forming structure that fits them very effectively.

In yet another alternative form, the seal-forming structure may be customised to an individual patient. The customisation may take the patient's facial features into consideration, especially in and around the target sealing regions.

In one form, the shapes and size of seal-forming structures may be based on one or more principal components of the seal-forming structure as determined from principal component analysis (PCA) conducted in relation to the shape and size of nasal regions for different members of a population.

In one form, one of the principal components may relate to the size of the target sealing region. If the target sealing region is the nasal region, the size of the nose is relevant.

In another form, one of the principal components may relate to the degree of curvature of the target sealing region. If the target sealing region is the nasal region, the principal component relates to the degree of curvature of the nares.

In another form, one of the principal components may relate to overall shape and/or proportion of the facial features in the sealing region. If the target sealing region is the nasal region, the principal component relates to shape and/or proportion of the nose which may include length and/or breadth of the nose.

In one form, the patient interface further comprises a connection port configured, in use, to be connected to an air circuit and to convey the flow of breathable gas from the air circuit to the plenum chamber through the plenum chamber inlet port.

An aspect of one form of the present technology is a patient interface comprising a seal-forming structure which further comprises at least one adhesive surface having areas of different magnitudes of adhesion strength. In one form, a first region of the at least one adhesive surface may adhere to the patient's face during use with a greater adhesion strength than a second region of the at least one adhesive surface.

In one form, areas of the seal-forming structure which are configured to seal with sensitive regions of the target sealing region may have lower magnitudes of adhesion strength. A sensitive region may include, but is not limited to, regions which are more likely to be subject to skin trauma on repeated application and removal of the seal-forming structure.

In another form, areas of the seal-forming structure which are configured to seal with those regions of the target sealing region which are difficult to seal with may have higher magnitudes of adhesion strength.

In another form, areas of the seal-forming structure which are configured to seal with regions of the target sealing region which are both sensitive and difficult to seal with may have balanced magnitudes of adhesion strength.

In one aspect, there is provided a patient interface comprising a seal-forming structure having a thickness in the range of approximately 0.2 mm to 0.3 mm when the patient interface is worn by the patient.

In one aspect, there is provided a patient interface comprising a seal-forming structure comprising an adhesive surface provided with an adhesive that is suitable for at least one re-application of the seal-forming structure on the patient's face

An aspect of one form of the present technology is a patient interface further comprising a decoupling structure configured to at least partly decouple the seal-forming structure from the air circuit.

One aspect of the technology provides a patient interface for use in delivering breathable gas to a patient. The patient interface may comprise a plenum chamber pressurisable to a therapeutic pressure of at least 6 cmH₂O above ambient air pressure. The plenum chamber may comprise a plenum chamber inlet port configured to receive a flow of breathable gas at the therapeutic pressure for breathing by the patient. The patient interface may further comprise a seal-forming structure provided to the plenum chamber. The seal-forming structure may be configured to form a seal with a region of the patient's face surrounding an entrance to the patient's airways. The seal-forming structure may have an opening therein such that the flow of breathable gas is delivered to at least an entrance to the patient's nares. The seal-forming structure may be configured to maintain said therapeutic pressure in the plenum chamber throughout the patient's respiratory cycle in use. The seal-forming structure may comprise at least one adhesive surface configured in use to adhere to the region of the patient's face surrounding the entrance to the patient's airways to form the seal. The patient interface may further comprise a vent structure to allow a continuous flow of gases exhaled by the patient from an interior of the plenum chamber to ambient. The vent structure may be configured to maintain the therapeutic pressure in the plenum chamber in use. The patient interface may further comprise a connection port configured, in use, to be connected to an air circuit and to convey the flow of breathable gas from the air circuit to the plenum chamber through the plenum chamber inlet port. The patient interface may further comprise a decoupling structure configured to at least partly decouple the seal-forming structure from the air circuit.

In one form, the decoupling structure comprises a deformable component configured to deform when an end of the air circuit connected to the connection port is caused to tilt relative to the plenum chamber.

The deformable component can insulate the patient interface from forces incident on the air circuit, especially if the forces are greater than a pre-determined magnitude. By insulating the patient interface from excessive forces, it is ensured that the seal-forming structure does not get ripped off forcefully from the patient's face.

In one form, the deformable component is the plenum chamber.

In one form, the plenum chamber may have one or more areas which are made of a flexible material such as, but not limited to, thermoplastic elastomers (TPE) or silicone.

In one form, the patient interface comprises a tube having a first end and a second end, wherein the first end is fluidly connected to the plenum chamber inlet port and the second end comprises the connection port, and wherein the tube comprises the deformable component.

In one form, the tube comprises a flexible section configured to be more flexible than another section of the tube, wherein the deformable component comprises the flexible section.

In one form, the flexible section of the tube has a narrower diameter than another section of the tube.

In one form, the decoupling structure comprises a ball joint provided between the air circuit and the plenum chamber.

In one form, the ball joint may be a quick-release ball-joint which comprises a ball and a socket. The ball and the socket may be configured to disengage on application of a force greater than a pre-determined magnitude on the air circuit.

In one form, the decoupling structure comprises a first magnetic member provided to the connection port, the first magnetic member being configured to be magnetically coupled to a second magnetic member provided to the air circuit when the air circuit is connected to the connection port.

In one form, the first magnetic member separates from the second magnetic member when the air circuit is pulled away from the patient interface with a force greater than a magnetic force of attraction between the first magnetic member and the second magnetic member.

In one form, the first magnetic member is a magnet. In another form, the first magnetic member is a non-magnetised ferromagnetic material.

In one form, the first magnetic member is ring-shaped and encircles the connection port.

In certain forms, the patient interface comprises an adapter having a central bore to allow the passage of gas therethrough from a first end to a second end. The first end may be configured to fluidly connect directly or indirectly to the air circuit and the second end may be configured to be removably inserted into the plenum chamber inlet port. In an alternative form, the second end may be configured to connect to an anterior surface of the plenum chamber in use. The first end may be configured to magnetically couple with the air circuit (or a component that is itself coupled to the air circuit).

In some forms, the adapter may comprise a heat and moisture exchanger.

An aspect of one form of the present technology is a patient interface further comprising a seal-forming structure having a flange which has an outer surface and an inner surface, wherein the outer surface is on an opposite side of the flange to the inner surface, and wherein the adhesive surface is provided on the outer surface of the flange.

In one form, the flange may form a curved shape and pressure from the gases in the plenum chamber is incident on the inner surface.

An aspect of one form of the present technology is a patient interface further comprising a heat and moisture exchanger configured to capture humidity from gases exhaled by the patient and deliver humidity to the flow of breathable gas for breathing by the patient.

In one form, the heat and moisture exchanger comprises a body of heat and moisture exchanging material provided on an inside surface of the plenum chamber.

In one form, the patient interface comprises a tube having a first end and a second end, wherein the first end is fluidly connected to the plenum chamber inlet port and the second end comprises the connection port, and wherein the heat and moisture exchanger comprises a body of heat and moisture exchanging material provided inside the tube.

In one form, the patient interface comprises a heat and moisture exchanger distanced from the plenum chamber of the patient interface in use. For example, the patient interface may comprise a conduit fluidly connecting the heat and moisture exchanger with the plenum chamber. In some forms, the patient interface may comprise a heat and moisture exchange module comprising the heat and moisture exchanger. The heat and moisture exchange module may further comprise a vent.

An aspect of one form of the present technology is a patient interface which comprises a seal-forming structure and a plenum chamber which are integrally formed as a single component.

In one form, the seal-forming structure and the plenum chamber may be formed from the same material.

In another form, the seal-forming structure and the plenum chamber may be formed from different materials.

An aspect of one form of the present technology is a patient interface further comprising one or more adhesive tape sections provided to the seal-forming structure to form the at least one adhesive surface.

An aspect of one form of the present technology is a patient interface further comprising nasal prongs configured to engage with the patient's nares and to convey the flow of breathable gas into the patient's nares.

In one form, the nasal prongs may include a couple of nasal pillows, each of which is configured to engage with one nare of the patient.

An aspect of one form of the present technology is a patient interface comprising a seal-forming structure which further comprises at least one adhesive surface configured in use to adhere to the region of the patient's face surrounding the entrance to the patient's airways to form the seal, wherein the patient interface further comprises a positioning guide member configured to facilitate positioning of the patient interface against the patient's face in use.

In one form, the positioning guide member may comprise a member having a concave portion facing towards the patient's face. For example, the member may comprise a V-shaped member which is configured to engage with the nasal septum when the patient interface is positioned correctly against the patient's face.

In one form, the positioning guide member may form part of the plenum chamber. The positioning guide member may be formed from a soft and pliable material that allows the patient to wear a patient interface with the positioning guide member continuously, for a long period, with little discomfort.

In another form, the positioning guide member may be provided removably to the plenum chamber. The positioning guide member may be removed from the patient interface after positioning the patient interface.

An aspect of one form of the present technology is a patient interface further comprising an applicator.

In one form, the applicator may be removably attached to an anterior surface of the seal-forming structure and/or the plenum chamber. The applicator may be configured to assist in positioning the patient interface in a therapeutically effective position on the patient's face before being removed.

In one form, the applicator may be adhered to the anterior surface of the seal-forming structure and/or the plenum chamber.

In one form, a surface of the applicator may be removably attached to the anterior surface of the seal-forming structure and/or the plenum chamber.

In another form, the applicator may be shaped to complement the region of the patient's face with which the seal-forming structure is configured to form a seal.

An aspect of one form of the present technology is a patient interface further comprising a removable layer removably attached to, and covering, the adhesive surface.

In one form, the removable layer may be configured to be removed prior to the patient interface being positioned in sealing contact with the patient's face.

In one form, the removable layer comprises a tab which, when the removable layer is attached to the adhesive surface, extends beyond the perimeter of the seal-forming structure.

An aspect of one form of the present technology is a patient interface comprising a seal-forming structure which further comprises at least one adhesive surface configured in use to adhere to the region of the patient's face surrounding the entrance to the patient's airways to form the seal and one or more tabs for gripping the seal-forming structure, wherein the one or more tabs do not have an adhesive surface facing towards the patient.

In certain forms, the one or more tabs are located at a periphery of the seal-forming structure. For example, the one or more tabs may comprise a first tab located on a first side of the opening and a second tab located on a second side of the opening, the first side being opposite the second side.

An aspect of another form of the present technology is a patient interface comprising a seal-forming structure which further comprises at least one adhesive surface configured in use to adhere to the region of the patient's face surrounding the entrance to the patient's airways to form the seal, wherein the adhesive surface is configured such that the adhesive strength of the adhesive surface is reduced by the deformation of the adhesive surface, for example when the adhesive surface is stretched.

In certain forms, the seal-forming structure may comprise one or more tabs for gripping the seal-forming structure, wherein the one or more tabs do not have an adhesive surface facing towards the patient, and wherein the tabs are configured to be pulled in order to deform (e.g. stretch) the seal-forming structure and reduce the adhesive strength of the adhesive surface, for example to remove the seal-forming structure from the patient's face.

An aspect of one form of the present technology is a patient interface further comprising one or more shape retainers. The shape retainer may be configured to promote retention of the shape of a seal-forming structure and/or the plenum chamber before the seal-forming structure is made to adhere to the patient's face and optionally during use, i.e. after the seal-forming structure is made to adhere to the patient's face. For example, the shape retainer may assist in maintaining the shape of the seal-forming structure and/or plenum chamber to a sufficient extent to prevent the seal-forming structure and/or plenum chamber from crumpling, folding or sagging in a way that makes it difficult for the patient to affix the patient interface to their face.

In certain forms, the one or more shape retainers may be configured to substantially maintain the shape of the seal-forming structure after the seal-forming structure is made to adhere to the patient's face.

In certain forms, the seal-forming structure comprises the one or more shape retainers.

In one form, the shape retainer may be located on a side which in use faces away from the patient's face.

In certain forms, the one or more shape retainers comprise elongate members.

In one form, the shape retainer may be made of the same material as the seal-forming structure and/or plenum chamber. For example, the shape retainer may be integrally formed with the seal-forming structure and/or plenum chamber.

In another form, the shape retainer may comprise the one or more tabs.

In one form, the shape retainer may be configured to detach, e.g. be removed, from the seal-forming structure/plenum chamber once the patient interface is in position on the patient's face. In certain forms, the shape retainer may be relatively weakly adhered to the seal-forming structure/plenum chamber. In another form, the shape retainer may disconnect (e.g. delaminate) from the seal-forming structure/plenum chamber when force is applied on the seal-forming structure/plenum chamber in a certain direction.

In one form, the shape retainer may be configured such that the seal-forming structure can stretch in one direction more easily than it can stretch in a different direction.

An aspect of one form of the present technology is a patient interface system for use in delivering breathable gas to a patient. The patient interface system may comprise a patient interface. The patient interface may comprise a plenum chamber pressurisable to a therapeutic pressure of at least 6 cmH₂O above ambient air pressure. The plenum chamber may include a plenum chamber inlet port configured to receive a flow of breathable gas at the therapeutic pressure for breathing by the patient. The patient interface may further comprise at least one seal-forming structure configured to be provided to the plenum chamber in use. Each at least one seal-forming structure may be configured to form a seal with a region of the patient's face surrounding an entrance to the patient's airways. Each at least one seal-forming structure may have an opening therein such that the flow of breathable gas is delivered to at least an entrance to the patient's nares. Each at least one seal-forming structure may be configured to maintain said therapeutic pressure in the plenum chamber throughout the patient's respiratory cycle in use. Each at least one seal-forming structure may comprise at least one sealing surface configured in use to be adhered to the region of the patient's face surrounding the entrance to the patient's airways to form the seal. The patient interface may further comprise a vent structure to allow a continuous flow of gases exhaled by the patient from an interior of the plenum chamber to ambient. The vent structure may be configured to maintain the therapeutic pressure in the plenum chamber in use. The patient interface system may further comprise an adhesive for applying to the at least one sealing surface of the at least one seal-forming structure of the patient interface.

In certain forms, the patient interface may further include a diffuser configured to diffuse the vent flow of gases from the interior of the plenum chamber to ambient.

An aspect of one form of the present technology is a patient interface of any one or more of the other aspects of the technology, and further comprising a second vent configured to vent received air before the air is supplied to the patient interface. The second vent may be at a different location in the patient interface from the first vent.

In one form, the patient interface comprises a tube, the tube comprising a first end configured to connect to the patient interface and a second end configured to connect to an air circuit. In one form, the first vent may be located at or proximate the first end of the tube. In one form, the second vent may be located at or proximate the second end of the tube.

In one form, the adhesive may be a fluid.

An aspect of one form of the present technology is a patient interface system further comprising one or more adhesive tape sections configured to be provided to the at least one sealing surface of the at least one seal-forming structure.

An aspect of one form of the present technology is a patient interface system further comprising a first seal-forming structure comprising a first sealing surface. The first sealing surface may be configured to adhere to a first region of the patient's face. The patient interface system may further comprise a second seal-forming structure comprising a second sealing surface. The second sealing surface may be configured to adhere to a second region of the patient's face, the first region being different from the second region.

In one form, the first seal-forming structure and the second seal-forming structure are configured to be selectively interchangeably provided to the plenum chamber.

In one form, the first sealing surface and the second sealing surface are configured such that the second region of the patient's face surrounds the first region of the patient's face.

In one form, the first region comprises the alar rim, a superior region of the lip superior and, optionally, the columella, of the patient's face.

In one form, the second region comprises the alar, a region of the upper lip inferior to the upper region and the pronasale.

An aspect of one form of the present technology is a patient interface system further comprising an applicator which may be configured to assist in positioning the patient interface in a therapeutically effective position on the patient's face.

In one form, the applicator comprises a body comprising a recess configured to receive and retain the patient interface while the patient interface is put into the therapeutically effective position on the patient's face.

In one form, the body of the applicator comprises a surface shaped to complement the region of the patient's face with which the seal-forming structure is configured to form a seal, wherein the surface is provided around the recess to support the seal-forming structure while the patient interface is put into the therapeutically effective position on the patient's face.

In one form, the applicator comprises a gripping portion provided to the body, the gripping portion being configured to be held by the patient, or another user, while the patient interface is put into the therapeutically effective position on the patient's face.

In one form, the applicator is configured to be removably attached to an anterior surface of the patient interface to assist in positioning the patient interface in the therapeutically effective position on the patient's face before being removed.

In one form, the applicator is removably attached to the anterior surface by an adhesive.

In another form, the applicator may be magnetically coupled to the anterior surface.

In one form, the body comprises two prongs extending out of the recess, the prongs configured to be inserted into the nares of the patient while the patient interface is put into the therapeutically effective position on the patient's face.

An aspect of one form of the present technology is a method of customizing a patient interface for use in delivering breathable gas to a patient. The method may comprise receiving data indicative of a shape and/or size of a region of the patient's face surrounding an entrance to the patient's airways with which the patient interface is to form a seal. The method may further comprise selecting a pre-formed patient interface assembly. The pre-formed patient interface assembly may comprise a plenum chamber pressurisable to a therapeutic pressure of at least 6 cmH₂O above ambient air pressure. The plenum chamber may include a plenum chamber inlet port configured to receive a flow of breathable gas at the therapeutic pressure for breathing by the patient. The pre-formed patient interface assembly may further comprise a seal-forming structure provided to the plenum chamber. The seal-forming structure may be configured to form a seal with the region of the patient's face, said seal-forming structure having an opening therein such that the flow of breathable gas is delivered to at least an entrance to the patient's nares, the seal-forming structure being configured to maintain said therapeutic pressure in the plenum chamber throughout the patient's respiratory cycle in use. The seal-forming structure may comprise at least one adhesive surface configured in use to adhere to the region of the patient's face surrounding the entrance to the patient's airways to form the seal. The method may further comprise shaping and/or sizing the seal-forming structure to complement the region of the patient's face, the shaping and/or sizing being performed on the basis of the data indicative of the shape and/or size of the region of the patient's face.

In one form, selecting the pre-formed patient interface assembly comprises selecting the selected pre-formed patient interface out of a plurality of pre-formed patient interfaces, wherein each of the plurality of pre-formed patient interfaces has a different shape and/or size.

In one form, shaping the seal-forming structure comprises inserting the seal-forming structure in a molding device.

In one form, the molding device is a thermo-forming device and shaping the seal-forming structure comprises thermo-forming the seal-forming structure.

In one form, the method further comprises forming a mold part of the molding device using the data.

In one form, the method further comprises scanning the region of the patient's face in order to generate the data.

An aspect of one form of the present technology is a molding device which is configured to manufacture a plurality of patient interfaces.

In one form, the molding device may be configured to receive a sheet of material, for example tape, from which one or more seal-forming structures for the patient interfaces are configured to be cut.

In one form, the molding device may include a cavity component which configured to interact with a core component to shape the tape into the desired shape for the seal-forming structure.

In one form, the cavity component and the core component are configured to have a gap between them when they are brought together in the shaping process. In one form, molten overmold material may be injected into the gap to overmold a plenum chamber onto the seal-forming structure.

In one form, the molding machine may include cutting apparatus to detach each patient interface from the tape.

According to one aspect of the present technology, there is provided a patient interface. The patient interface may comprise a plenum chamber pressurisable to a therapeutic pressure above ambient pressure. The patient interface may further comprise a seal-forming structure provided to the plenum chamber. The seal-forming structure may be configured to form a seal with a region of the patient's face surrounding an entrance to the patient's airways. The seal-forming structure may be configured to maintain said therapeutic pressure in the plenum chamber throughout the patient's respiratory cycle in use. The seal-forming structure may comprise at least one adhesive surface configured in use to adhere to the region of the patient's face surrounding the entrance to the patient's airways to form the seal. The patient interface may further comprise a vent structure to allow a flow of gas from ambient to an interior of the plenum chamber for breathing by the patient, and to allow a flow of gases exhaled by the patient from the interior of the plenum chamber to ambient. The vent structure may be configured to maintain the therapeutic pressure in the plenum chamber in use.

In certain forms, the vent structure may be configured to create and maintain the therapeutic pressure in the plenum chamber from the flow of gases exhaled by the patient. Such a vent may be referred to as an expiratory resistance valve and therapy using such a vent may be referred to as expiratory positive airway pressure (EPAP).

According to one aspect of the technology, there is provided a patient interface. The patient interface may comprise a plenum chamber. The patient interface may further comprise a seal-forming structure provided to the plenum chamber. The seal-forming structure may be configured to form a seal with a region of the patient's face surrounding an entrance to the patient's airways. The seal-forming structure may comprise at least one adhesive surface configured in use to adhere to the region of the patient's face surrounding the entrance to the patient's airways to form the seal. The patient interface may further comprise a vent structure to allow a flow of gas from ambient to an interior of the plenum chamber for breathing by the patient, and to allow a flow of gases exhaled by the patient from the interior of the plenum chamber to ambient. The patient interface may further comprise a sensor module configured to detect characteristics of the patient's breathing and/or other characteristics of the patient's health and/or physiology.

In certain forms, the characteristics of the patient's breathing detected by the sensor module may be used to determine a medical condition of the patient, for example to diagnose a respiratory condition, e.g. obstructive sleep apnea (OSA).

According to one aspect of the technology, there is provided a respiratory diagnosis system comprising a patient interface. The patient interface may comprise a plenum chamber. The patient interface may further comprise a seal-forming structure provided to the plenum chamber. The seal-forming structure may be configured to form a seal with a region of the patient's face surrounding an entrance to the patient's airways. The seal-forming structure may comprise at least one adhesive surface configured in use to adhere to the region of the patient's face surrounding the entrance to the patient's airways to form the seal. The patient interface may further comprise a vent structure to allow a flow of gas from ambient to an interior of the plenum chamber for breathing by the patient, and to allow a flow of gases exhaled by the patient from the interior of the plenum chamber to ambient. The patient interface may further comprise a sensor module configured to detect characteristics of the patient's breathing and/or other characteristics of the patient's health and/or physiology.

An aspect of certain forms of the present technology is a medical device that is easy to use, e.g. by a person who does not have medical training, by a person who has limited dexterity, vision or by a person with limited experience in using this type of medical device.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

FIG. 1 shows a system including a patient 1000 wearing a patient interface 3000, receiving a supply of air at positive pressure from an RPT device 4000. The patient is sleeping lying on their side.

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoid and trapezius.

FIG. 2L shows an anterolateral view of a nose.

FIG. 3A shows a patient interface which is configured to adhere to an alar rim region of a patient's face in accordance with one form of the present technology.

FIG. 3B shows a side perspective view of a seal-forming structure of the patient interface of FIG. 3A (left) and a side perspective view of a nose showing the region of the face to which the seal-forming structure of FIG. 3A adheres in use (right).

FIG. 3C shows a front perspective view of the seal-forming structure of the patient interface of FIG. 3A.

FIG. 4A shows a perspective view of a patient interface which is affixed to a patient's face in accordance with one form of the present technology.

FIG. 4B shows a side view of the patient interface of FIG. 4A.

FIG. 4C shows a front view of the patient interface of FIG. 4A.

FIG. 4D shows a side perspective view of a nose showing the region of the face to which the seal-forming structure of FIG. 4A adheres in use.

FIG. 5A a perspective view of a patient interface which is affixed to an alar crease region of a patient's face in accordance with one form of the present technology.

FIG. 5B shows a front view of a seal-forming structure of the patient interface of FIG. 5A.

FIG. 5C shows a rear view of the patient interface of FIG. 5A.

FIG. 6 shows a cross-sectional view of a seal-forming structure according to another form of the technology.

FIG. 7 shows regions to which a first seal-forming structure and a second seal-forming structure of a patient interface system may adhere in accordance with one form of the present technology.

FIG. 8 is a perspective view illustration of a patient interface in accordance with another form of the present technology.

FIG. 9A is a perspective view illustration of a patient interface in accordance with another form of the present technology.

FIG. 9B is an exploded view illustration of the patient interface of FIG. 9A.

FIG. 9C shows a front view of the patient interface of FIG. 9A as positioned on the patient's face.

FIG. 9D shows a front view of the patient interface of FIG. 9A when the seal-forming structure adheres to the patient's face.

FIG. 10A shows an exploded view of a patient interface system.

FIG. 10B shows an exploded view of a patient interface system having a magnetic coupling structure in accordance with another form of the present technology.

FIG. 10C shows an exploded view of a tube and an air circuit of the patient interface system of FIG. 10B.

FIG. 10D shows a cross-sectional view of the tube and the air circuit of the patient interface system of FIG. 10B when assembled.

FIG. 10E shows a front view of the patient wearing the patient interface, wherein the air circuit of the patient interface system of FIG. 10B is detached from the patient interface.

FIG. 10F shows a side view of a patient lying on their side while wearing a patient interface according to one form of the technology.

FIG. 11 shows a cross-sectional view of a patient interface having a heat and moisture exchanger in accordance with one form of the present technology.

FIG. 12A illustrates a molding tool and patient interface according to one form of the present technology.

FIG. 12B shows the patient interface of FIG. 12A.

FIG. 12C shows a plurality of pre-formed patient interfaces according to another form of the technology.

FIG. 13A shows a patient interface system in accordance with one form of the present technology. The patient interface system includes a patient interface having a seal-forming structure, and an applicator which is configured to transfer the seal-forming structure to the patient's face.

FIG. 13B shows the arrangement of the seal-forming structure and the applicator before the seal-forming structure is transferred to the patient's face.

FIG. 13C shows the arrangement of the seal-forming structure and the applicator on transferring the seal-forming structure to the patient's face and releasing the applicator from the seal-forming structure.

FIG. 14A shows a patient interface system in accordance with one form of the present technology. The patient interface system includes a patient interface having a seal-forming structure, a removable layer, and a front layer.

FIG. 14B shows the arrangement of the components of the patient interface system of FIG. 14A when the patient interface is being transferred to the patient's face.

FIG. 14C shows the arrangement of the components of the patient interface system of FIG. 14A after transferring the patient interface to the patient's face.

FIG. 15A shows a rear view, a side view and a front view of a patient interface which includes a removable layer.

FIG. 15B shows an exploded view of a patient interface comprising a front layer with an extension in accordance with one form of the present technology.

FIG. 15C shows a perspective view of a patient interface of FIG. 15B when assembled.

FIGS. 16A-16F are plan view illustrations of seal-forming structures according to certain forms of the technology.

FIG. 17 is an exploded view illustration of a patient interface and applicator according to one form of the technology.

FIGS. 18A-18C are illustrations of applicators according to further forms of the technology.

FIG. 19A is a perspective view illustration of a patient interface which includes a positioning guide member according to one form of the technology.

FIG. 19B is a front view of a patient positioning the patient interface illustrated in FIG. 19A.

FIG. 19C is a front view illustration of a patient interface which includes a positioning guide member according to another form of the technology.

FIG. 19D is a perspective view illustration of the patient interface illustrated in FIG. 19C.

FIG. 19E is a cross-sectional view illustration of a patient interface which includes a positioning guide member according to another form of the technology.

FIG. 20A is an exploded cross-sectional view of a patient interface and an air circuit according to one form of the technology.

FIG. 20B is a perspective partial view of a seal-forming structure according to one form of the technology.

FIG. 20C is an exploded partial view of a patient interface and an air circuit according to one form of the technology.

FIG. 20D is a cross-sectional view of an adapter according to one form of the technology.

FIG. 20E is a rear perspective view of an adapter according to one form of the technology.

FIG. 20F is a front perspective view of an adapter according to one form of the technology.

FIG. 21A is a perspective view of an adhesive tape according to one form of the technology.

FIG. 21B illustrates an injection molding tool and patient interface blank according to one form of the present technology.

FIG. 21C illustrates the injection molding tool of FIG. 21B molding the patient interface blank.

FIG. 21D illustrates a cross-sectional view of FIG. 21C.

FIG. 22A illustrates a perspective view of a patient interface having shape retainers according to one form of the technology.

FIG. 22B illustrates a bottom perspective view of the patient interface of FIG. 22A.

FIG. 22C illustrates a top view of the patient interface of FIG. 22A when force is applied on its ends.

FIG. 23A illustrates a perspective view of a molding machine according to one form of the technology.

FIG. 23B illustrates an enlarged perspective view of a tape when the molding machine of FIG. 23A is in use according to one form of the technology.

FIG. 24A illustrates a disassembled view of the tape according to one form of the technology.

FIG. 24B illustrates a perspective view of a portion of the tape of FIG. 24A.

FIG. 25A illustrates one step of a manufacturing process for forming a patient interface according to one form of the technology.

FIG. 25B illustrates another step of the manufacturing process for forming a patient interface according to one form of the technology.

FIG. 25C illustrates another step of the manufacturing process for forming a patient interface according to one form of the technology.

FIG. 25D illustrates another step of the manufacturing process for forming a patient interface according to one form of the technology.

FIG. 25E illustrates another step of the manufacturing process for forming a patient interface according to one form of the technology.

FIG. 26 illustrates a front view of a patient interface according to one form of the technology.

FIG. 27 illustrates an exploded sectional view of a part of an adhesive seal-forming structure according to one form of the technology.

FIG. 28 illustrates a perspective view of a respiratory therapy system comprising a split-vent according to one form of the technology.

FIG. 29 illustrates a perspective view of a patient interface system according to one form of the technology.

FIG. 30 illustrates a perspective view of a patient interface and an expiratory resistance module according to one form of the technology.

FIG. 31 illustrates a perspective view of a patient interface and a sensor module according to one form of the technology.

FIG. 32 illustrates a perspective view of a respiratory therapy system according to one form of the technology.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

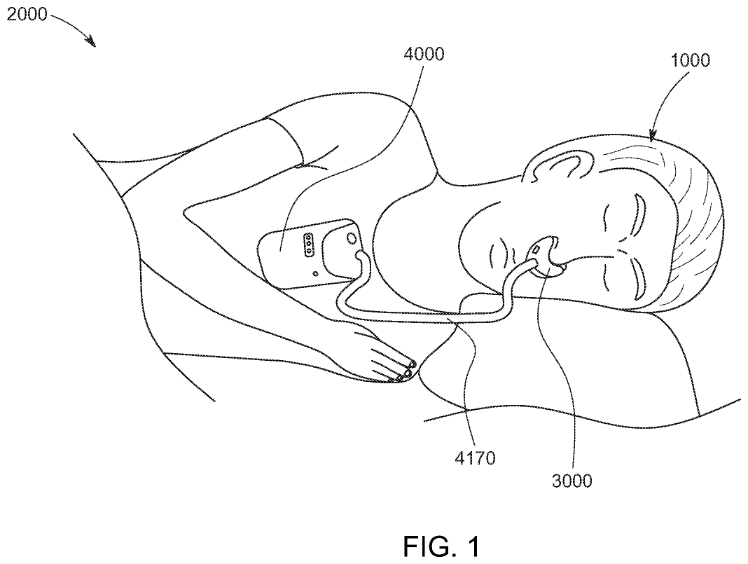

In one form, as shown in FIG. 1, the present technology comprises a respiratory therapy system 2000 for treating a respiratory disorder. The respiratory therapy system 2000 may comprise an RPT device 4000 for supplying a flow of air to the patient 1000 via an air circuit 4170 and a patient interface 3000.

In the form of the technology shown in FIG. 1, the RPT device 4000 is portable and can be carried by the patient 1000, for example attached to the patient's clothing. In an alternative form of the present technology (not shown in the Figs.), the RPT device may be a conventional RPT device configured to rest on a nearby surface during use, for example a bedside table.

Further, the respiratory therapy system 2000 may include a humidifier (not shown in the Figs.) to change the absolute humidity of air or gas for delivery to a patient relative to ambient air. Typically, the humidifier is used to increase the absolute humidity and increase the temperature of the flow of air (relative to ambient air) before delivery to the patient's airways.

In certain forms of the technology, the patient interface 3000 may comprise a heat and moisture exchanger 3700 as explained in more detail below.

### 4.3 Patient Interface

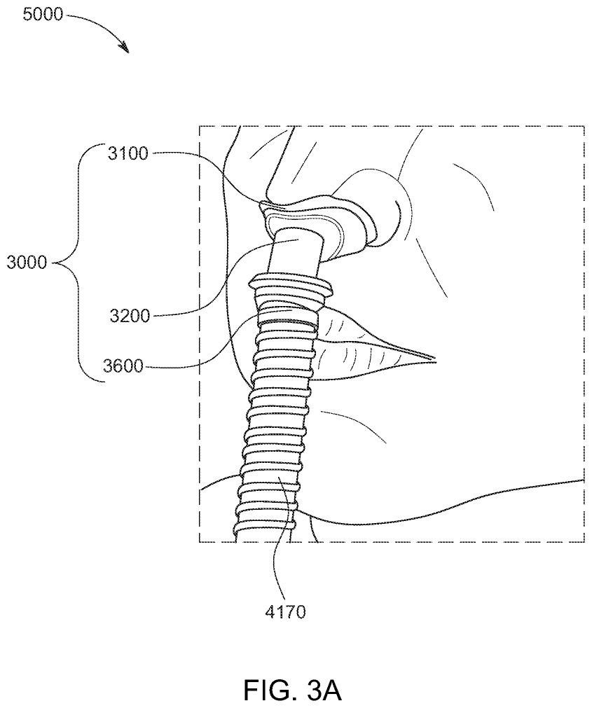

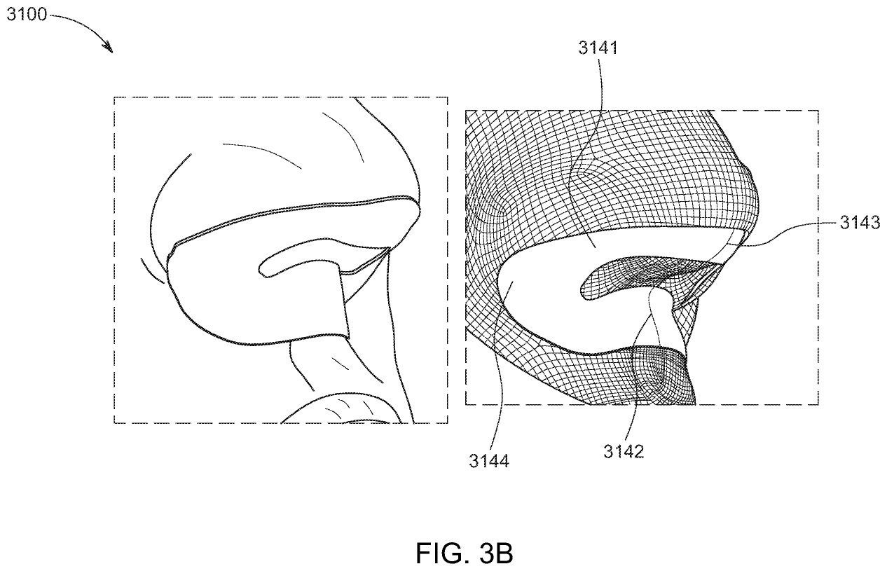

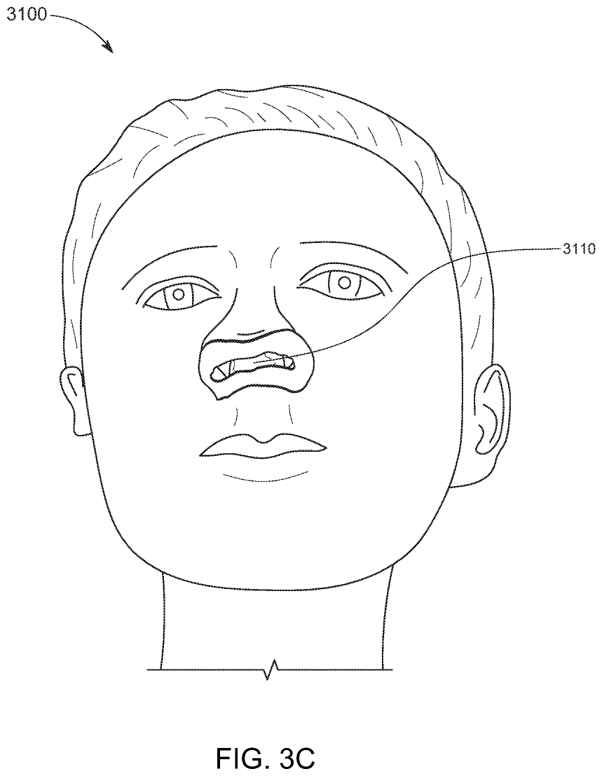

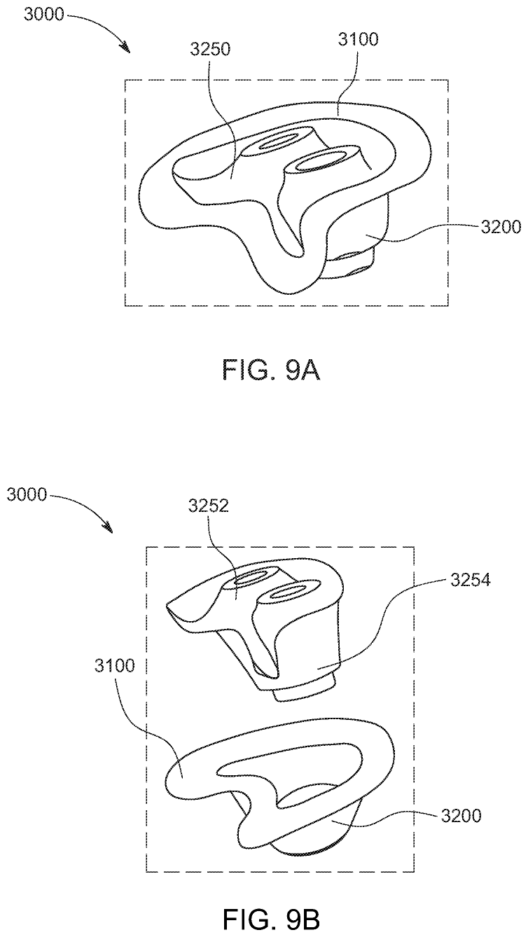

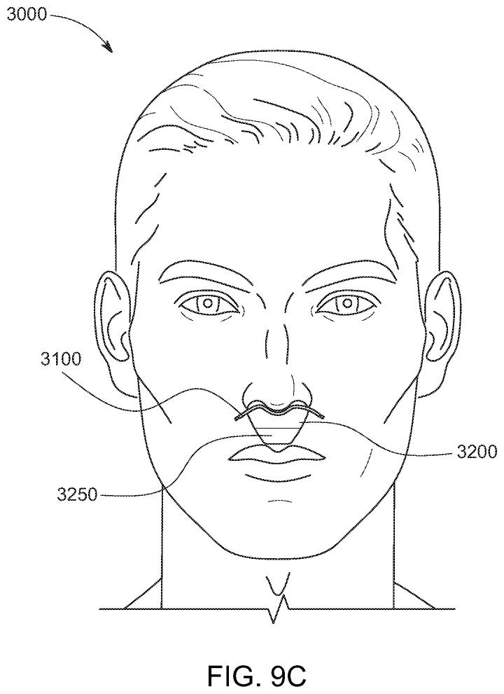

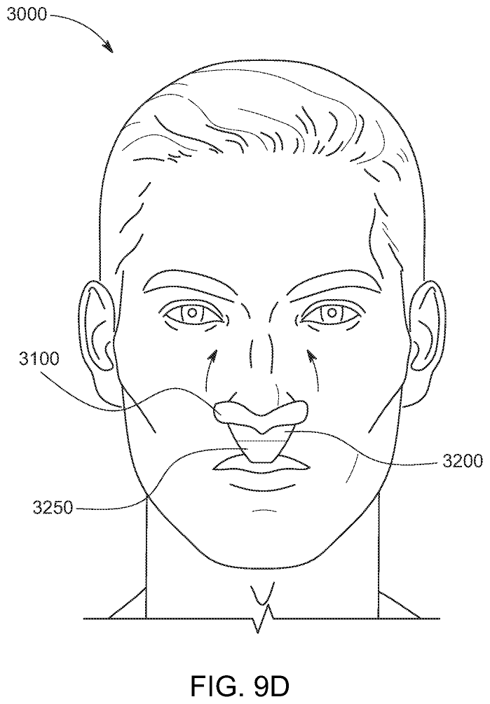

FIGS. 3A to 8, 10A to 18C, 20A to 22C and 26 to 32 show forms of the technology providing a patient interface 3000 (or parts thereof) in which the patient interface 3000 does not extend into the nares of the patient 1000. In the form of technology shown in FIGS. 9A to 9D, patient interface 3000 comprises nasal prongs that engage with and/or extend into the nares. These types of patient interfaces will be described in more detail in the following paragraphs.

A patient interface 3000, such as shown in FIGS. 3A, in accordance with one aspect of the present technology comprises the following functional aspects: a seal-forming structure 3100, a plenum chamber 3200, and a connection port 3600 for connection to an air circuit 4170.

In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

The plenum chamber 3200 may be formed of one or more modular components in the sense that it or they can be replaced with different components, for example components of a different size and/or shape.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 6 cmH₂O with respect to ambient.

The patient interface 3000 in accordance with one form of the present technology is constructed and arranged to be able to provide a supply of air at a positive pressure of at least 10 cmH₂O with respect to ambient.

The patient interface 3000 in accordance with one form of the present technology is constructed and arranged to be able to provide a supply of air at a positive pressure of at least 20 cmH₂O with respect to ambient.

### 4.3.1 Seal-Forming Structure

In one form of the present technology, the patient interface 3000 includes a seal-forming structure 3100 which is configured to form a seal with a region of the patient's face. The seal-forming structure 3100 is thereby configured to secure the plenum chamber 3200 in a sealing engagement with respect to the patient's face. The seal-forming structure 3100 may form an opening 3110 to allow a flow of breathable gas to be delivered to at least an entrance to the patient's nares.

In one form of the present technology, the seal-forming structure 3100 provides a target seal-forming region. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, and the shape of a patient's face.

The seal-forming structure 3100 of certain forms of the technology is configured to adhere, through an adhesive, to one or more regions of the patient's face so as to form a seal with a region of the patient's face surrounding an entrance to one or more of the patient's airways. For example, the seal-forming structures 3100 in FIGS. 3A to 32 are each configured to seal around the nasal airways of the patient 1000. However, in alternative forms of the present technology (not shown in the Figs.) the seal-forming structure may be configured to seal around the mouth of the patient 1000. The seal-forming structure may also be configured to seal with both the nasal airways and the mouth of the patient 1000.

The adhesive-based attachment of the seal-forming structure 3100 to the patient's face allows formation of a highly airtight seal. An adhesive-based seal has been found to allow far less leak than can be achieved with conventional compression seals held on the face by headgear. One reason for this is that, in forms where the seal-forming structure 3100 that is adhered to the patient's face is sufficiently flexible that it can distort dynamically with the patient's face, the seal is maintained even if the patient's face or skin moves. Such movement can cause leaks to occur with conventional compression seals. The shape of the face can particularly change as the patient moves from an upright posture to a lying down posture, and also between lying down positions (e.g. on the back (supine), on the front (prone), or on the side).

A high quality seal improves the effectiveness of positive pressure respiratory therapy since the desired pressure can be maintained in the patient interface. Furthermore, a high quality seal reduces the overall power required to be expended by an RPT device 4000 to maintain the pressure of the breathable gas in the patient interface 3000. This reduced power requirement means that RPT devices which run on relatively low power, such as a portable RPT device, can be used. This makes it easier for a patient to receive therapy when they are in situations other than their home, for example while travelling. Also, reduced power requirements may reduce the cost and complexity of the RPT device 4000.

With conventional compression seals, there is a trade-off between comfort and leakage. A greater retaining force holding the patient interface on the face (typically exerted by the pull of headgear straps) reduces the risk of leakage but makes the patient interface less comfortable to wear, and more likely to result in marks on the patient's face. In case of infants and children, use of headgear over an extended time may lead to development of deformities in the face and/or the skull which may have highly significant consequences.

On the other hand, if the retaining force is too low, leakage will occur, particularly at high therapy pressures. Furthermore, the level of interference that appropriately balances these considerations may vary around the perimeter of the seal for any given patient interface, and may also vary from patient to patient because of variations in an individual's facial shape. Adhesive-based seals may avoid these difficulties since the seal may be able to more effectively follow the shape of the patient's face, particularly if the seal-forming structure is sufficiently flexible, and to adhere to the skin with a consistent retaining force around the perimeter of the seal-forming structure.

Preventing leakage is advantageous in oxygen therapy, especially in countries, regions or periods of time when oxygen availability is quite scarce.

Moreover, the absence of headgear improves patient comfort significantly. This improves the overall efficacy of the respiratory treatment because the patient 1000 will tend to not adjust or otherwise move a patient interface 3000 which is comfortable.

In particular, children and infants typically have a lower tolerance for discomfort than adults. Consequently, they tend to remove or repeatedly attempt to remove the headgear during use. If a patient interface with headgear is disengaged or repeatedly disturbed during use, it may significantly affect the efficacy of the treatment or therapy. The seal-forming structure 3100 of forms of the technology described in this specification provides a more comfortable and therefore effective seal in contrast to headgear. Moreover, the patient interface 3000 with the seal-forming structure 3100 provides a safer means to administer respiratory therapy to children and infants because the use of headgear can be dispensed with, thereby eliminating application of force across the skull by the headgear.

It has also been observed that adhesive-based attachment of the seal-forming structure 3100 to the patient's face leads to less occlusion of the patient's nares than may be experienced with some other types of patient interface. As will be described, the seal-forming structure 3100 of exemplary forms of the technology are configured to adhere around the alar rim of the patient's nose. This means that substantially the whole of the nostril area is available for receiving incoming gas. Furthermore, in forms of the technology in which the seal-forming structure 3100 is able to flex with the patient's face, the nose is free to balloon slightly as a result of the incoming pressured gas, which increases the area of the nares further. These effects make it easier for a patient to breathe when using patient interfaces 3000 according to the forms of the technology described herein compared to some conventional patient interfaces. For example, patient interfaces that use compression seals may push inward on the nares, making it more difficult for them to expand outwardly, and some patient interfaces are structured so that both the inside and outside surfaces of the nose are contained within the pressurised plenum chamber. This means there is a lack of pressure differential between the inner and outer surfaces of the nose, so no ballooning effect occurs. In contrast, some forms of the technology described herein create a seal around the alar rim so that there is a pressure differential between the inner and outer surfaces of the nares, enabling this ballooning effect. This benefit may be particularly noticeable for patients with narrow nares.

### 4.3.1.1 Adhesive Surface

The seal-forming structure 3100 is configured to be secured in a therapeutically effective position against the patient's face through an adhesive. In some forms of the present technology the seal-forming structure may be configured to adhere to the patient's face through vacuum-induced attachment in addition to the one or more adhesives.

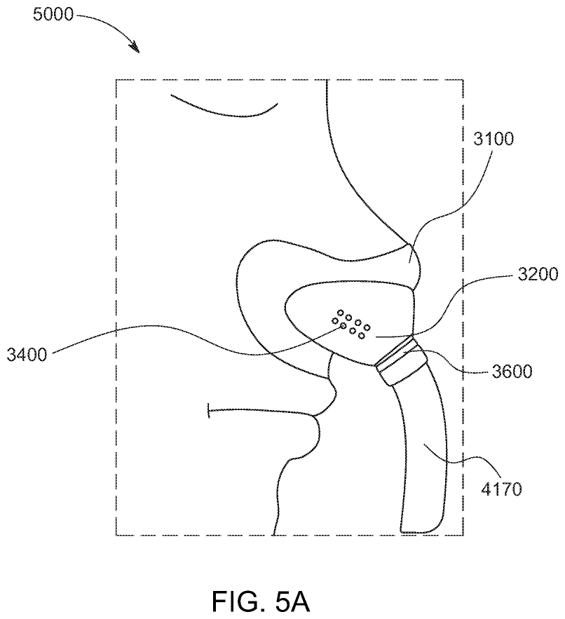

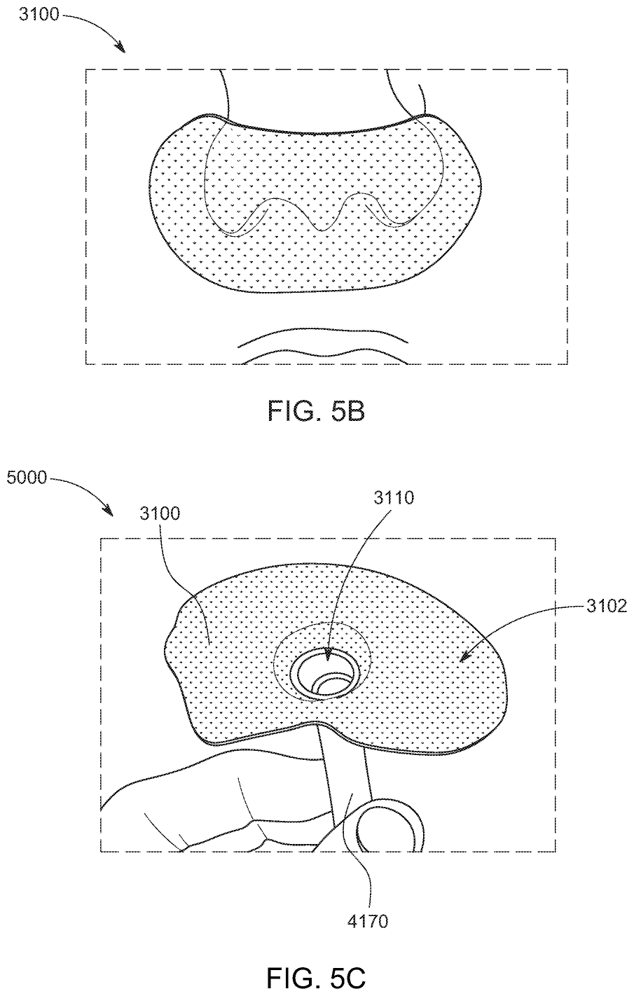

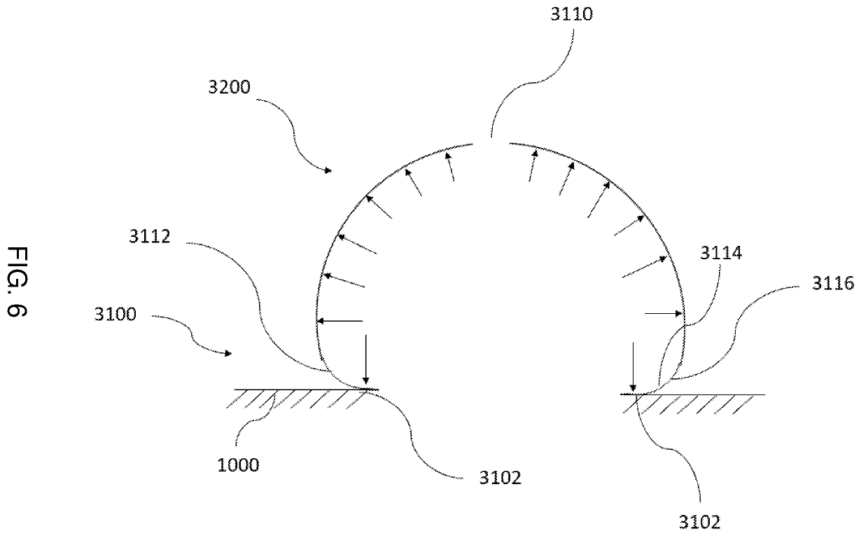

In one form, the seal-forming structure 3100 comprises a region having at least one adhesive surface 3102. An exemplary adhesive surface 3102 is illustrated in FIG. 5C in one form of the technology.

The adhesive is applied to the adhesive surface 3102, and in use, the adhesive surface 3102 contacts the patient's face, so that the adhesive adheres the adhesive surface 3102 of the seal-forming structure 3100 to the patient's face.

In certain forms of the present technology, the seal-forming structure 3100 is configured so that the shape of the adhesive surface 3102 substantially matches or resembles the shape of the region of the patient's face to which the seal-forming structure 3100 is in use attached. As will be explained in more detail later, in certain forms the seal-forming structure 3100 may be configured to substantially match/resemble the shape of a region of a particular patient's face, i.e. the seal-forming structure 3100 may be customised for an individual patient. Customising the seal-forming structure 3100 in patient interfaces 3000 of the types described herein may be more commercially viable than is the case with other types of patient interface because the small footprint of the seal-forming structure 3100 makes them a relatively small component and the seal-forming structure 3100 may be able to be cut out of a flat piece of material, such as adhesive tape, reducing the relative costs of manufacture for an individual patient. Alternatively, the seal-forming structure 3100 may be configured with a shape that substantially complements the shape of the appropriate target region of a generic face, or a generic face of a sub-group of the population (e.g. based on size, or type of facial shape). Alternatively, the seal-forming structure 3100 may be formed of a material and in a form that makes the seal-forming structure 3100 sufficiently flexible that it can adopt the shape of the region of the patient's face to which it is attached in use.

One advantage of the seal-forming structure 3100 and/or the adhesive surface 3102 being shaped to substantially match/resemble the region of the patient's face to which the seal-forming structure 3100 adheres, or to be sufficiently flexible that it is able to deform to do so, is that this avoids the adhesive surface 3102 pulling on the underlying skin when the patient interface 3000 is in use. This improves patient comfort significantly by eliminating uneven stresses developed by the adhesive exerting shear stresses on the skin.

Further, it may also allow a smaller amount of adhesive to provide the adhesive force required to secure the seal-forming structure 3100 against the patient's face than would otherwise be necessary because the adhesive does not exert force on pulling the skin laterally across the face.

The region of the seal-forming structure 3100 to which the adhesive is applied may be considered to be the target seal-forming region. In one form the adhesive is located on a ring-shaped region around an outer edge of the seal-forming structure 3100 with the ring-shaped region entirely surrounding the opening 3110. This may minimize the footprint of the seal-forming structure 3100 on the patient's face, thereby reducing the bulk of the patient interface 3000 and its inconvenience to the patient 1000.

The close proximity of the seal-forming structure 3100 and the presence of the adhesive on the surface of the skin may cause sweat from the underlying skin to accumulate and/or interact with the adhesive. The accumulation of sweat may be inconvenient when the patient interface 3000 is to be worn over an extended period of time, such as during sleep. Therefore, the smaller the area of the target seal-forming region and/or the area covered by the seal-forming structure 3100, the lesser is the inconvenience caused to a patient 1000.

### 4.3.1.2 Region of Patient's Face

The seal-forming structure forms a seal in use with a region of the patient's face surrounding an entrance of the patient's airways, for example surrounding the nares and/or the mouth.

There are several considerations in determining which region of the patient's face the seal-forming structure 3100 according to certain forms of the technology may be configured to attach to in use.

One factor is that it may be desirable for the seal-forming structure 3100 to have a small footprint on the patient's face. The larger the footprint, the more obtrusive the patient interface 3000. A patient interface 3000 occupying a large footprint may lead to feelings of claustrophobia and discomfort. A small footprint also typically means a smaller patient interface 3000, which uses less materials and is consequently cheaper to manufacture.

Similarly, the smaller the area of attachment between the adhesive surface 3102 and the underlying skin, the better it is for patient comfort since there is less area of skin that may react to having the seal-forming structure 3100 attached to it and less area of skin that can be pulled in different directions by the adhesive.

Moreover, since a smaller footprint of the seal-forming structure 3100 typically means a smaller patient interface, and consequently a smaller plenum chamber, this corresponds to a smaller area against which the pressure of the air in the plenum chamber 3200 acts. For any given pressure, a smaller area therefore leads to a reduced force which acts to push the patient interface 3000 off the patient's face. Consequently, a smaller footprint means that the adhesive does not need to be so strong to retain the patient interface 3000 on the patient's face.

Another advantage to having a seal-forming structure 3100 with a relatively small footprint is that, again since this typically means a smaller patient interface 3000, the patient interface 3000 does not tend to protrude too far away from the patient's face. This eliminates or reduces the likelihood of frictional interaction between the patient interface 3000 and surrounding objects such as pillowcases, counterpanes, etc. Certain regions of the patient's face are more likely to contact surrounding objects when a patient 1000 is asleep. Such regions include the cheeks, the chin, the pronasale and other such prominent features of the patient's face. If the patient interface 3000 protrudes out significantly from those regions of the patient's face, then the likelihood of the seal-forming structure 3100 peeling away from the skin slightly or completely is high. This is particularly so when the patient interface 3000 is worn for prolonged periods of sleep.

Certain areas of the face are prone to greater change in shape when a patient 1000 changes their body position, for example from sitting upright to lying down or between any of the following lying down positions: supine; prone; lying on the side. This is because, in the different positions, the force of gravity acts on the patient's skin and flesh differently, which causes them to move in different ways relative to the underlying bone structure. A seal-forming structure 3100 which avoids areas of greater variation in shape with change in a patient's body position provides greater comfort because variation in shape of the underlying facial regions while the patient interface 3000 is being worn will cause the adhesive surface 3102 to either stretch to accommodate change in shape or disengage with the underlying skin. If the adhesive surface 3102 and/or the seal-forming structure stretches, then a restorative force is exerted on the underlying skin, thereby causing discomfort to the patient 1000. If the seal-forming structure 3100 disengages from the patient's face, it could lead to ineffective sealing, and consequently ineffective respiratory therapy.

Some facial areas tend to have more facial hair than others, particularly in men. A seal-forming structure 3100 whose adhesive surface 3102 is configured to be attached to predominantly non-hairy portions allows application and removal of the seal-forming structure without causing pain due to hair removal. Pain caused by the yanking of facial hairs may discourage patients from adhering to respiratory therapy. Therefore, a seal-forming structure that adheres to non-hairy or less hairy regions of the patient's face is advantageous.

In addition to the above factors, the region of the patient's face to which the seal-forming structure 3100 attaches is also a factor when considering the shape of a seal-forming structure 3100 which is able to fit a number of members of a population, as opposed to being custom-made for an individual patient. Forming patient interfaces that are able to be used effectively by multiple members of the population may reduce manufacturing costs by enabling economies of scale.

Based on this consideration, extensive analysis has been undertaken to understand the extent of variation of different regions of the face across members of the population. In particular, Principal Component Analysis (PCA) of 3D facial scans of the regions around the airways of many members of the population was conducted. This analysis identified that certain regions of the face around the airways were found to vary to a lesser degree across a population as opposed to other regions.

In certain forms of the technology, the shape of the seal-forming structure 3100 is configured in such a way that it may be attached in use to these facial areas of lesser relative variation of shape. The following sections describe the regions of the face that have been identified as being suitable in this analysis, and shapes of seal-forming structures according to certain forms of the technology that are particularly suited to sealing with these regions.

In general, the regions of the face identified as being suitable for seal-forming structure 3100 of certain forms of the technology to adhere to, based on consideration of the factors described above, are regions adjacent to the nares, for example regions immediately adjacent to the nares. Such regions are the generally inferiorly facing surfaces of the nasal alar and a superior region of the lip superior, for example the superior-most region of the lip superior.

In one form of the technology, as shown in FIGS. 3A to 3C, the seal-forming structure 3100 is configured to adhere to regions of the patient's face immediately surrounding the nares. These regions are indicated by the non-hashed region in the right-hand side of FIG. 3B and comprise: the alar rim region 3141 (i.e. regions of the ala that are immediately adjacent the nares and may be generally inferiorly facing); the superior-most region of the lip superior 3142, which may comprise the subnasale and/or the region immediately inferior of the subnasale; and an anterior region of the nose that is inferior, e.g. immediately inferior, to the pronasale 3143. In the lateral direction, the seal-forming structure 3100 extends to a region 3144 slightly inferior to the alar crest point, for example a region immediately medial to the junction between the alar crest point and the nasolabial sulcus. In the form of the technology shown, the seal-forming structure 3100 does not adhere to a significant part of the side regions of the nasal alar, although in some forms, or for some faces, it may adhere to the inferior regions of the side regions of the nasal alar. Furthermore, the seal-forming structure 3100 of FIGS. 3A to 3C does not adhere to the pronasale.

In the form of the technology shown in FIGS. 3A to 3C, the region of the patient's face to which the seal-forming structure 3100 adheres is a band entirely surrounding both the patient's nares. The band may be approximately constant in width around the perimeter of the band.

The region of the face covered by the seal-forming structure 3100 in the form of the technology shown in FIGS. 3A to 3C has been found not to change shape substantially when a patient 1000 changes their position because this facial region predominantly comprises of cartilage and bone and has relatively little adipose tissue, as compared to the cheek or chin areas. This facial region is also typically free of facial hair or has very little facial hair (for example, some facial hair may be present on the superior-most region of the lip superior 3142). A seal-forming structure 3100 that adheres to this region may be particularly advantageous for a patient 1000 with upper lip hair.

The region of the face covered by the seal-forming structure 3100 in the form of the technology shown in FIGS. 3A to 3C has also been found to have relatively little variation in shape across patients in representative population samples, including patients of a variety of races.

Further, the size of this region (which may be referred to as the alar rim region) is small in size since it immediately surrounds the nares.

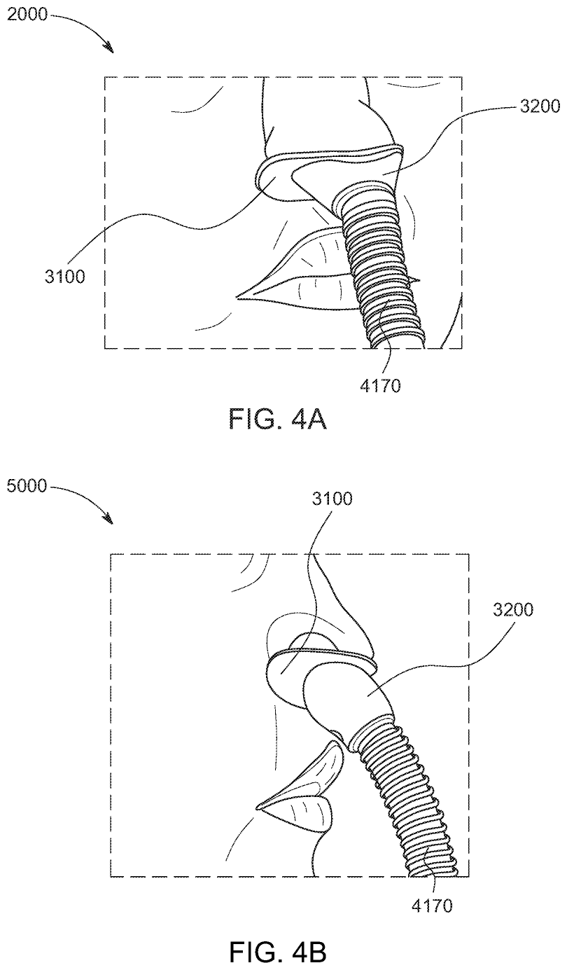

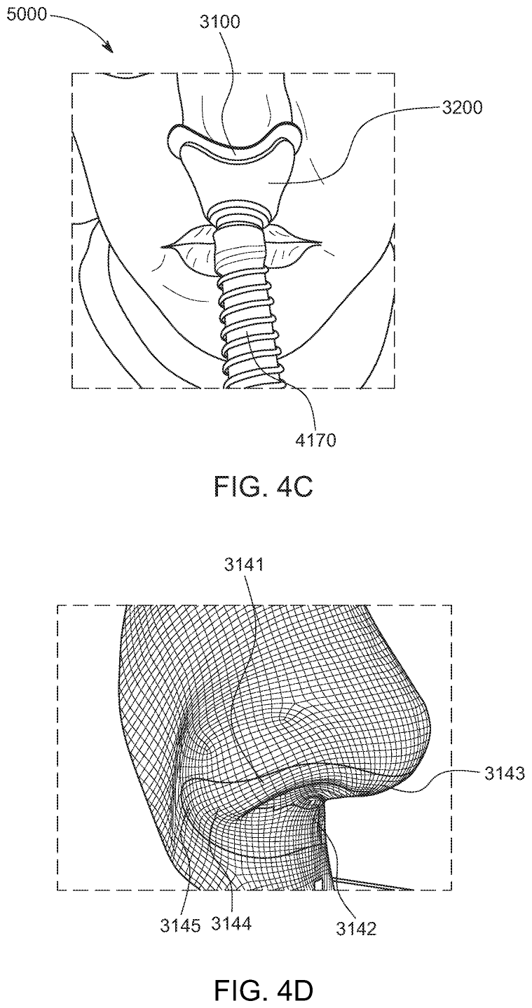

In another exemplary form, as shown in FIGS. 4A to 4D, the seal-forming structure 3100 is configured to adhere to a facial region that extends more superiorly compared to the region shown in FIG. 3B and includes the side regions of the nasal alar. In some forms, the seal-forming structure 3100 is configured to adhere to a side region of the nasal alar and may also extend radially outwardly sufficiently far to adhere in use to regions of the cheeks adjacent the nasal alar crest point 3145, e.g. the regions between the nasal alar and the nasolabial sulcus. This seal-forming structure 3100 may also adhere to the regions of the face to which the seal-forming structure of FIGS. 3A to 3C adheres. The larger area of adhesion of the seal-forming structure in FIGS. 4A to 4D may improve the amount of adhesion and result in fewer leaks compared to the seal-forming structure of FIGS. 3A to 3C, but may result in more discomfort for the patient.

In another exemplary form, as shown in FIGS. 5A to 5C, the seal-forming structure 3100 is configured to adhere to a larger region of the patient's face than the region to which the seal-forming structures of FIGS. 3A to 3C and 4A to 4D adhere. In this form the seal-forming structure adheres to a region that extends in a superior direction up to the alar crease region, i.e. when the seal-forming structure is adhered to the patient's face, a superior-most portion of the seal-forming structure adheres to the patient's alar crease. The seal-forming structure 3100 in this form may additionally, or alternatively, adhere to a substantial portion of the lip superior. Furthermore, the seal-forming structure 3100 in this form may additionally, or alternatively, adhere to the pronasale region, which may include a point which is immediately superior to the pronasale. In the lateral direction, the seal-forming structure 3100 in this form adheres to a region of the patient's cheeks adjacent to the nasal alar, including the regions between the nasal alar and the nasolabial sulcus, and the seal-forming structure 3100 may cover the nasal alar completely in use. This seal-forming structure 3100 may also adhere to the regions of the face to which the seal-forming structures of FIGS. 3A to 3C and/or FIGS. 4A to 4D adhere. Alternatively, the seal-forming structure of FIGS. 5A to 5C may be configured to adhere to some regions positioned radially outwardly from the nares compared to some regions to which the seal-forming structures of FIGS. 3A to 3C and/or FIGS. 4A to 4D adhere.

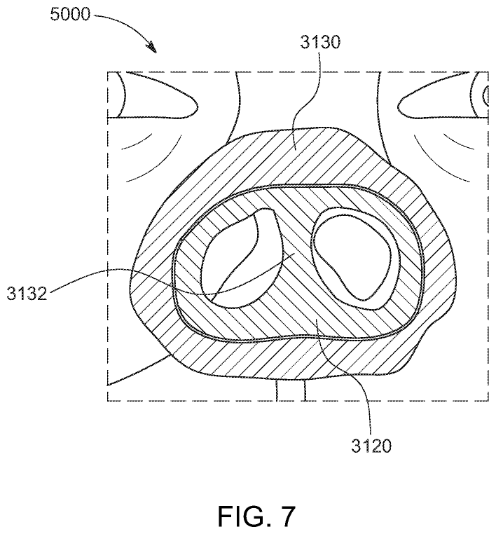

In certain forms of the technology, for example the form of the technology shown in FIG. 7, the seal-forming structure 3100 may be configured to adhere to the columella. For example, the seal-forming structure 3100 may comprise a septum region 3132 extending between two diametrically opposing regions of the seal-forming structure 3100, the septum region 3132 being configured to adhere in use to the columella.

### 4.3.1.3 Adhesion Strength Based on the Region of the Patient's Face

Certain regions of the face may be more sensitive than others to adhesives and/or the trauma caused by the skin being pulled at when the seal-forming structure 3100 is removed. In certain forms, there are provided seal-forming structures 3100 which have regions of the adhesive surface having different adhesion strengths depending upon the specific region of the face to which a particular part of the seal-forming structure 3100 is configured to adhere.

It may not be possible to achieve an air-tight seal while avoiding all or most of the sensitive regions of the patient's face. The seal-forming structure 3100 having different adhesion strength across the adhesive surface 3102 allows the seal-forming structure 3100 to adhere relatively strongly to relatively less sensitive regions of the patient's face, and with relatively less adhesion strength to the more sensitive regions of the face. The advantage of such an arrangement is that the load of bringing about an effective seal can be undertaken by the relatively less sensitive regions of the patient's face while avoiding acute skin trauma in the more sensitive regions.

Similarly, certain regions of the face are more difficult to achieve effective sealing with than others. Regions like the lip superior, for instance, are more difficult for the seal-forming structure 3100 to form an effective seal with because of the contours of the philtrum and the tendency for facial hair to grow here compared to relatively flatter, less hairy areas such as the cheeks. Adhesion strength of a greater magnitude may therefore be beneficial to seal more effectively in regions which are more difficult to seal with. Therefore, in one form, the adhesive surface of those parts of the seal-forming structure 3100 which are configured to adhere to the relatively difficult-to-seal-with-regions may possess greater adhesion strength than the adhesive of the other parts of the seal-forming structure 3100.

The alar crease is a region of the patient's face which is both tricky to form an effective seal with (because of the contours in the region), and is also a sensitive region. Therefore, the adhesive strength of a part of the seal-forming structure 3100 that seals with the alar crease may be selected to balance forming an effective seal but reducing trauma on the skin due to repeated application and removal of the seal-forming structure 3100.

Certain areas of the patient's face may be more suited for an effective seal to be formed with, and parts of a seal-forming structure 3100 contacting these areas may consequently require lesser adhesion strength. Examples of such areas include the regions of the patient's face on the side of the nose and cheeks region. Adhesion strength of parts of the seal-forming structure 3100 configured to seal to these areas may be either at a magnitude that is sufficient to bring about effective sealing, or it may be at a magnitude higher than necessary to compensate for lower adhesion strength in other regions.

In certain forms, a region of the adhesive surface 3102 of the seal-forming structure 3100 may be afforded greater adhesion strength by using a stronger adhesive in the region compared to another region. Alternatively, or additionally, in other forms, the greater adhesion strength of a region of the seal-forming structure 3100 may be provided by applying a greater amount of an adhesive, for example the same adhesive. In other forms there are provided other ways to vary the adhesion strength between different regions of the seal-forming structure 3100.

### 4.3.1.4 Exemplary Shapes of Seal-Forming Structure

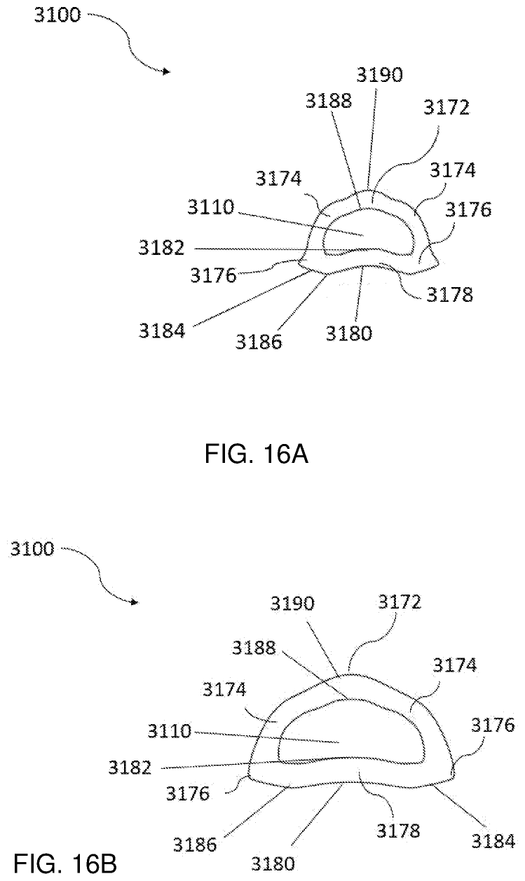

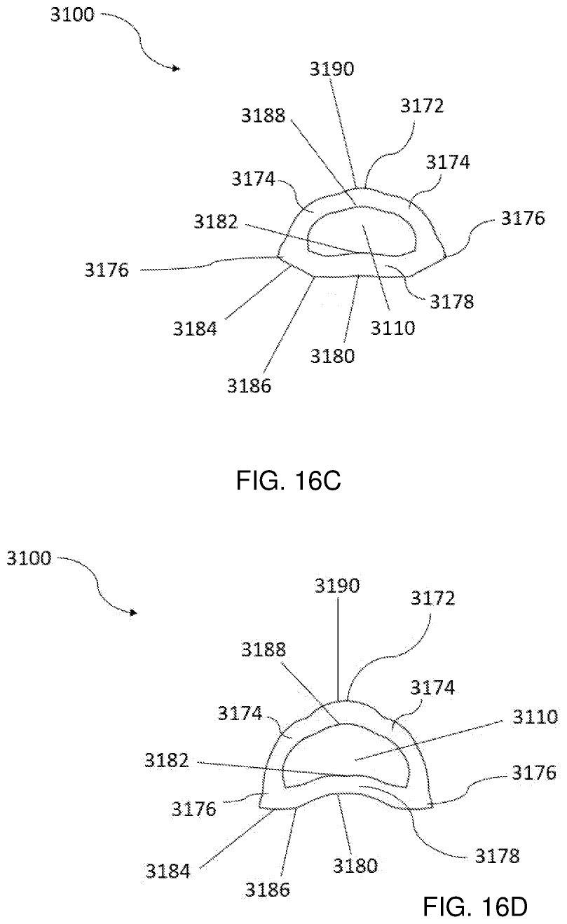

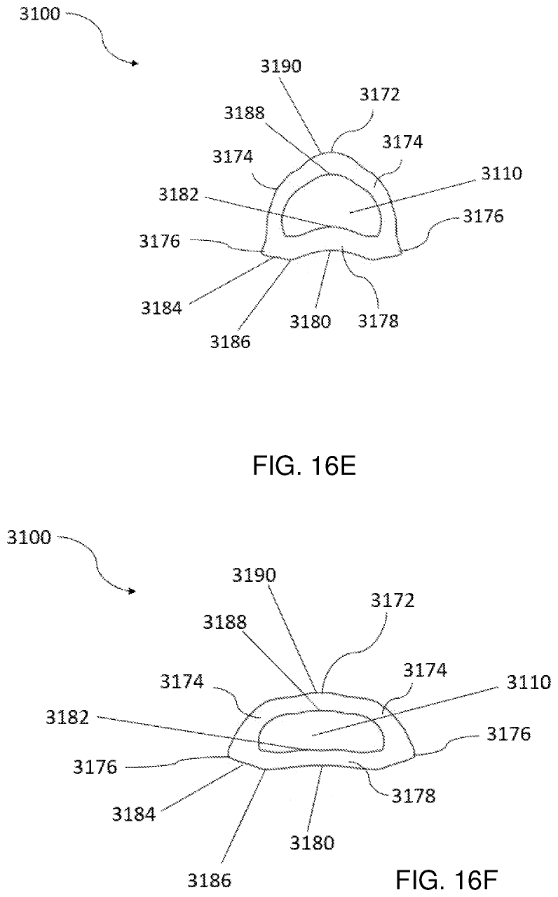

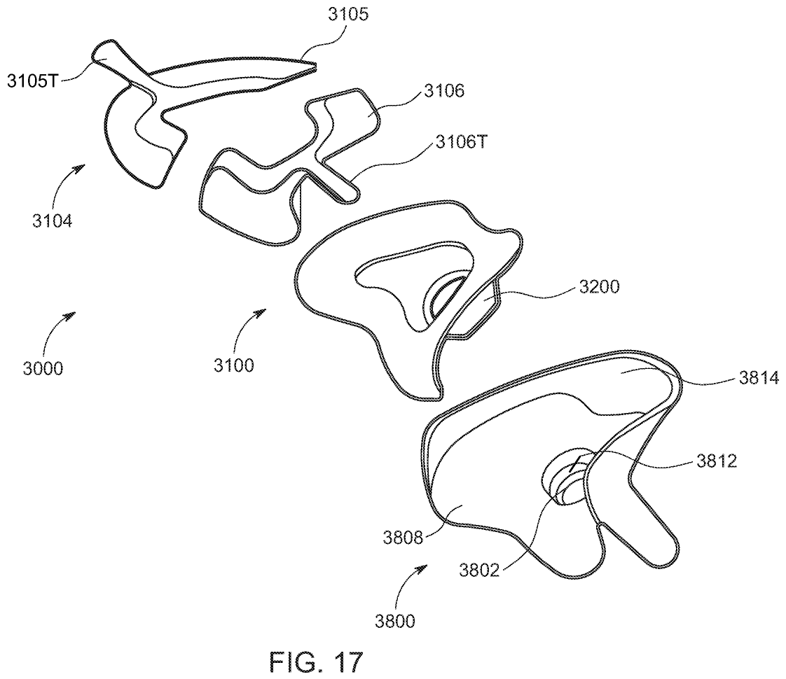

FIGS. 16A to 16F show exemplary seal-forming structures 3100 of certain forms of the technology which are shaped to be adhered to the highlighted region shown in FIG. 4D and described above.

### 4.3.1.4.1 General Shape of Seal-Forming Structures

The seal-forming structures 3100 shown in FIGS. 16A to 16F are formed of a sheet of material with an opening 3110 in the middle. The form of the seal-forming structures 3100 comprises a stem portion 3178, two corner portions 3176, two shoulder portions 3174 and an apex portion 3172. As shown in the FIGS. 16A to 16F, the corner portions 3176 are located at either end of the stem portion 3178, and the shoulder portions 3174 connect each corner portion 3176 to the apex portion 3172. Each of the seal-forming structures 3100 shown in FIGS. 16A to 16F has reflectional symmetry about an axis through the apex portion 3172 and a mid-section of the stem portion 3178. This axis is intended to be positioned on the patient's mid-sagittal plane in use.

The stem portion 3178 may in some forms be substantially straight, and in other forms may be arced radially inwardly such that the stem portion 3178 comprises a concave recess 3180 in a mid-section of its radially outer side and a convex protrusion 3182 in a mid-section of its radially inner side. The recess 3180 and the protrusion 3182 may be generally rounded in shape.

The corner portions 3176 may be generally triangular in shape and may each comprise an edge 3184 on the radially outer side on the same side of the seal-forming structure 3100 as the recess 3180 in the stem portion 3178. The edge 3184 may be angled to create an obtuse corner 3186 between the angled edge 3184 and the recess 3180 of the stem portion 3178.

The apex portion 3172 may comprise a concave recess 3188 on its radially inner side and a convex protrusion 3190 on its radially outer side. The recess 3188 and the protrusion 3190 may be generally rounded in shape.

The shoulder portions 3174 may have radially outer and/or radially inner edges that arc radially outwardly such that the shoulder portions 3174 comprise a convex protrusion on the radially outer edge and a concave recess on the radially inner edge.

In the form of technology shown in FIGS. 16A to 16F, the seal-forming structures 3100 generally have the form of a D-shaped band. In other forms of the technology, seal-forming structures 3100 formed from sheets may differ in shape, although may still comprise one or more of the portions described above.

The seal-forming structures shown in FIGS. 16A to 16F are configured to adhere, in use, to regions of the face such as those indicated by the outlined region in FIG. 4D. For example, in use, the apex portion 3172 is configured to be adhered to an anterior region of the nose that is inferior to the pronasale, the shoulder portions 3174 are configured to be adhered to inferior regions of the nasal alar, the corner portions 3176 are configured to be adhered to the alar crease region, for example, the alar crease and/or the laterally outer regions of the nasal alar and/or the regions of the cheek adjacent the alar crease, and the stem portion 3178 is configured to adhere to a superior region of the lip superior.

### 4.3.1.4.2 Variation in Shape of Seal-Forming Structures

FIGS. 16A to 16F show seal-forming structures 3100 in a plurality of shapes and/or sizes. Patient interfaces 3000 of the type described herein may be configured so that any one of the plurality of seal-forming structures 3100 may be able to be used as part of the patient interface, with the seal-forming structures 3100 being interchangeably connectable to the plenum chamber 3200 of the patient interface 3000. In this way, a patient may be able to select the seal-forming structure 3100 that is shaped and sized to best suit their needs, for example from a fit and comfort perspective.

The range of shapes of the seal-forming structures 3100 shown in FIGS. 16A to 16F may be selected so that at least one of the shapes is able to fit most patients in a given population of patients. This means that a patient interface is able to be configured to provide a good level of fit with only a discrete number of generic shapes and sizes of seal-forming structures 3100, for example six in the exemplary forms shown in FIGS. 16A to 16F. Producing only a discrete number of seal-forming structures 3100 (as compared to customising a seal-forming structure to fit an individual patient) may provide cost efficiencies in manufacturing and supply. It will be appreciated that, in other forms of the technology, a different number of different shapes and sizes of seal-forming structures 3100 may be provided. The greater the number of seal-forming structures 3100 provided, the more likely any individual patient is to be able to find a seal-forming structure that fits them very effectively, but the more different shapes of structure that are manufactured and supplied. In other forms of the technology, a seal-forming structure 3100 may be customised to an individual patient.

The shapes of the seal-forming structures 3100 shown in FIGS. 16A to 16F have been identified by taking a particular shape and size of seal-forming structure 3100 considered suitable for fitting a target region of a generically shaped and sized patient's face, and altering the shape and size based on changes to one or more principal components of the seal-forming structure as determined from PCA conducted in relation to the shape and size of nasal regions for different members of a population. For example, in the PCA conducted to generate the seal-forming structures 3100 shown in FIGS. 16A to 16F, it was determined that three main principal components of the target sealing region (such as shown in FIG. 4D) vary between members of a population, and the different shapes/sizes of seal-forming structures 3100 shown in FIGS. 16A to 16F are generated by independently varying each of these three principal components. More specifically, FIGS. 16A and 16B are generated by varying a first principal component by minus three standard deviations (FIG. 16A) and plus three standard deviations (FIG. 16B). FIGS. 16C and 16D are generated by varying a second principal component by minus three standard deviations (FIG. 16C) and plus three standard deviations (FIG. 16D). FIGS. 16E and 16F are generated by varying a third principal component by minus three standard deviations (FIG. 16E) and plus three standard deviations (FIG. 16F).

It should be appreciated that principal components identified in PCA do not necessarily equate to any particular characteristic or property of the size/shape of a region. Nevertheless, it may broadly be seen that the first principal component relates generally to the size of the target sealing region, and therefore the size of the nose, the second principal component relates generally to the degree of curvature of the nares, and the third principal component relates generally to the overall shape and/or proportion of the nose, for example whether the nose is long and thin or short and wide. In other forms of the technology, a range of seal-forming structures 3100 suitable for adhering to a patient's face may vary based on different principal components or by varying a plurality of principal components between each version of seal-forming structure.

In the exemplary forms of the seal-forming structures 3100 shown in FIGS. 16A and 16B, it can be seen that varying the first principal component varies the overall size of the seal-forming structure 3100, i.e. the seal-forming structure shown in FIG. 16B is a scaled-up version of the seal-forming structure shown in FIG. 16C. Furthermore, the seal-forming structure shown in FIG. 16B has wider obtuse corners 3186 compared to the seal-forming structure shown in FIG. 16A, i.e. the edge 3184 of the seal-forming structure shown in FIG. 16B is oriented more parallel to the outer edge of the stem portion 3178 than is the case in the seal-forming structure shown in FIG. 16A. Furthermore, the concave protrusion 3190 in the apex portion 3172 may be more pronounced in a seal-forming structure 3100 for a smaller nose (as per FIG. 16A) than for a larger nose (as per FIG. 16B).

In the exemplary forms of the seal-forming structures 3100 shown in FIGS. 16C and 16D, varying the second principal component results in a change in the degree of concavity and convexity of the recess 3180 and protrusion 3182 of the stem portion 3178 respectively. In the seal-forming structure shown in FIG. 16D, the amount of concavity and convexity in both cases is larger than that of the seal-forming structure shown in FIG. 16C, and consequently the shoulder portions 3174 are longer and the corner portions 3176 are located further from the apex portion 3172 than is the case with the seal-forming structure shown in FIG. 16C. For the seal-forming structure shown in FIG. 16C, the stem portion 3178 is substantially straight and the edges 3184 are angled relative to a line joining the two corner portions 3176. In contrast, the edges 3184 of the seal-forming structure shown in FIG. 16D are parallel to a line joining the two corner portions 3176. The seal-forming structure shown in FIG. 16C may be better suited for faces with relatively flat nares while the seal-forming structure shown in FIG. 16D may be better suited for faces with relatively curved nares.

In the exemplary forms of the seal-forming structures 3100 shown in FIGS. 16E and 16F, varying the third principal component results in a change in the length of the seal-forming structure 3100 in a direction perpendicular to a line joining the two corner portions 3176, i.e. the distance separating the stem portion 3178 and the apex portion 3172. The seal-forming structure shown in FIG. 16E may be generally more suited to patients with longer and/or thinner noses while the seal-forming structure shown in FIG. 16F may be generally more suited to patients with shorter and/or wider noses. In the seal-forming structure shown in FIG. 16E, the apex portion 3172 is close to the stem portion 3178 compared to that of seal-forming structure shown in FIG. 16F, i.e. the seal-forming structure 3100 of FIG. 16E is more stretched in a direction parallel to its axis of reflectional symmetry than the seal-forming structure shown in FIG. 16F. 4.3.1.4.3 Other Exemplary Shapes of Seal-Forming Structures

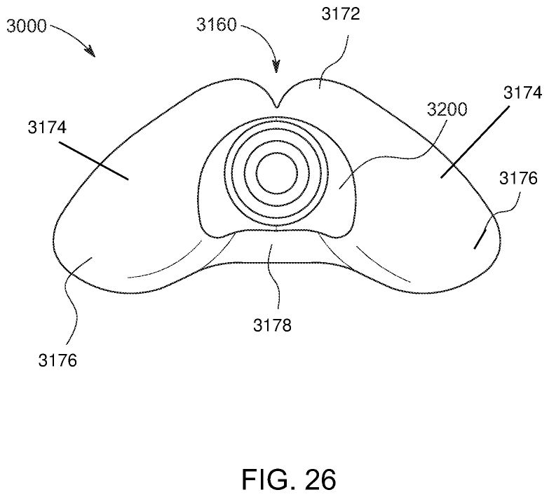

FIG. 26 illustrates a patient interface 3000 comprising a seal-forming structure 3100 and a plenum chamber 3200 according to another form of the technology. Similar to the seal-forming structures of FIGS. 16A-16F, the seal-forming structure 3100 of FIG. 26 comprises an apex portion 3172, first and second corner portions 3176 and a stem portion 3178. The seal-forming structure 3100 also comprises shoulder portions 3174 between the apex portion 3172 and the respective corner portions 3176.

When the patient interface 3000 of FIG. 26 is worn by a patient, the apex portion 3172 is configured to adhere to a region of the patient's nose substantially inferior to the pronasale of the patient 1000. In some forms, the apex portion 3172 may also adhere to the pronasale of the patient 1000. In some forms, the apex portion 3172 includes a notch 3160 in its radially outer edge. The notch 3160 is a region of the apex portion 3172 in which the radial distance from the outer side of the apex portion 3172 from the central opening in the seal-forming structure 3100 is less than adjacent portions. The outer side of the apex portion 3172 may curve inwardly to form notch 3160. When a seal-forming structure 3100 is affixed to the patient's face, the region of the seal-forming structure 3100 proximate the pronasale may crease because of an excess of material of the seal-forming structure 3100 converging in this region. The notch 3160 of the seal-forming structure 3100, as illustrated in FIG. 26, may assist in avoiding such creasing. Creasing of any part of the seal-forming structure 3100 may cause discomfort and/or form lines on the patient's face, especially if the seal-forming structure 3100 is worn for a long duration of time, such as overnight. Crease(s) in the seal-forming structure 3100 may also allow leaks, thereby rendering the seal partially or wholly ineffective and not air-tight, which may impact on the effectiveness of therapy. In other forms, the apex portion 3172 may be provided without notch 3160.

It should also be appreciated that a similar notch may be provided in other forms of seal-forming structure 3100, for example in the apex portion 3172 of seal-forming structures 3100 having different shapes, or in a portion of any seal-forming structure 3100 that is configured to adhere proximate to, or to, the pronasale in use.

The shoulder portions 3176 of the seal-forming structure 3100 of FIG. 26 may be configured to adhere substantially to the alar rim region 3141 of the patient 1000.

The stem potion 3178 may be configured to adhere to the lip superior 3142 of the patient's face, for example a superior portion of the lip superior.

The corner portions 3176 of the seal-forming structure 1000 in FIG. 26 are larger than the corner portions of the forms of the technology shown in FIGS. 16A-16F. The corner portions 3176 of the form of FIG. 26 may be described as wings of the seal-forming structure 3100. The corner portions 3176 may be configured to adhere in use to the patient's nasal alar on each side of the nose. In one form, the corner portions 3176 may be configured to extend, when worn, in a superior direction substantially up to the alar crease region on each side of the patient's face and in a lateral direction up to a region immediately medial to the junction between the alar crest point and the nasolabial sulcus. In other forms, the corner portions 3176 may also adhere to regions of the patient's face adjacent to the nasal alar, for example a region superior to the alar crease and/or regions of the patient's cheek adjacent to the nasal alar. For example, in one form, the corner portions 3176 may extended, when worn, in a lateral direction up to a region immediately lateral to the junction between the alar crest point and the nasolabial sulcus. It will be appreciated that the region of sealing for any given patient will depend on the size of the seal-forming structure 3100 relative to the patient's face, as well as the positioning of the seal-forming structure 3100 on the face.

### 4.3.1.5 Opening

The seal-forming structure 3100 includes at least one opening 3110 through which breathable gas is conveyed to the patient's airways. In one form, the opening 3110 may be a hole, as shown in FIGS. 3B, 3C, 5C and 16A. The hole may be generally D-shaped (for example as shown in FIGS. 16A to 16F) so that the seal-forming structure 3100 in which the hole is formed may adhere to a region of the patient's face around the nares. In other forms, the hole may be a different shape, for example substantially circular, oval or crescent-shaped.

### 4.3.1.6 Flange

The peripheral region of the seal-forming structure 3100 configured to be adhered to the patient's face in use may be considered to be a flange. The flange may have an inner surface and an outer surface, where the outer surface is on an opposite side of the flange to the inner surface. The inner surface is on the side of the flange that is contiguous with the inner surface of the plenum chamber 3200 and the outer surface is on the side of the flange that is contiguous with the outer surface of the plenum chamber 3200.

In the forms of the technology shown in FIGS. 3A to 5C and 8 to 11, for example, the adhesive surface is provided on the inner surface of the flange. That is, in use, the inner surface of the flange is adhered to the patient's face.

In the form of the technology shown in FIG. 6, the seal-forming structure 3100 includes a flange 3112. The flange 3112 includes an inner surface 3114 and an outer surface 3116. The adhesive surface 3102, in this form, is provided on the outer surface 3116 of the flange 3112. The outer surface 3116 may be a part of the outer surface of the seal-forming structure 3100. The flange 3112 is formed in way that it turns inwardly towards the internal volume inside plenum chamber 3200 and presents its outer surface 3116 towards the patient 1000 for adhesion to the patient's face.

The form of the technology shown in FIG. 6 may benefit from utilizing a pressure-assisted sealing mechanism. In use, the positive pressure of gas in the interior of the plenum chamber 3200 acts on the inner surface 3114 of the flange 3112, urging the outer surface 3116 into tighter sealing engagement with the face. Therefore, in such a form, the pressure of gas inside the plenum chamber 3200 may act to improve adhesion instead of acting to push the seal-forming structure 3100 away from the skin. This, in turn, may reduce the amount of adhesive force required to maintain the seal between the patient's face and the seal-forming structure 3100. The reduction in the required adhesive force may cause less removal of skin cells and less skin trauma despite repeated application and removal of the seal-forming structure 3100. It may additionally, or alternatively, mean that an adhesive of reduced strength, or less adhesive, be applied to the adhesive surface of the seal-forming structure 3100 in some forms.

### 4.3.1.7 Non-Adhesive Tabs

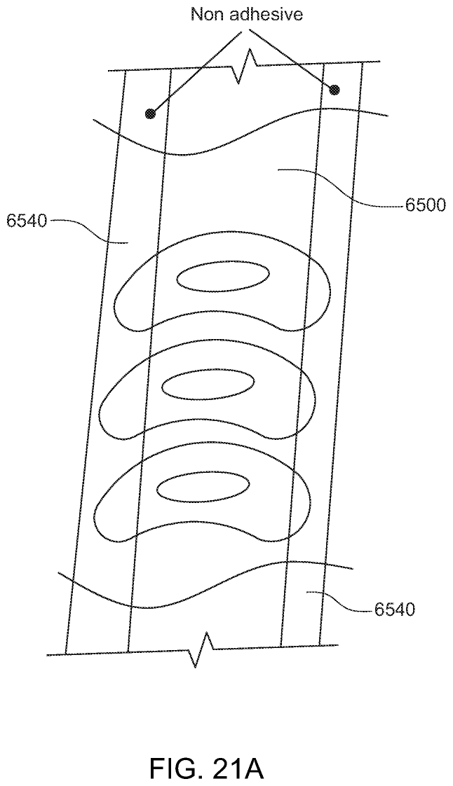

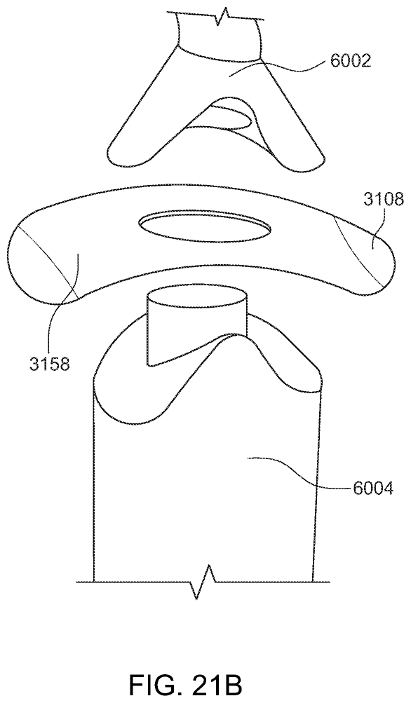

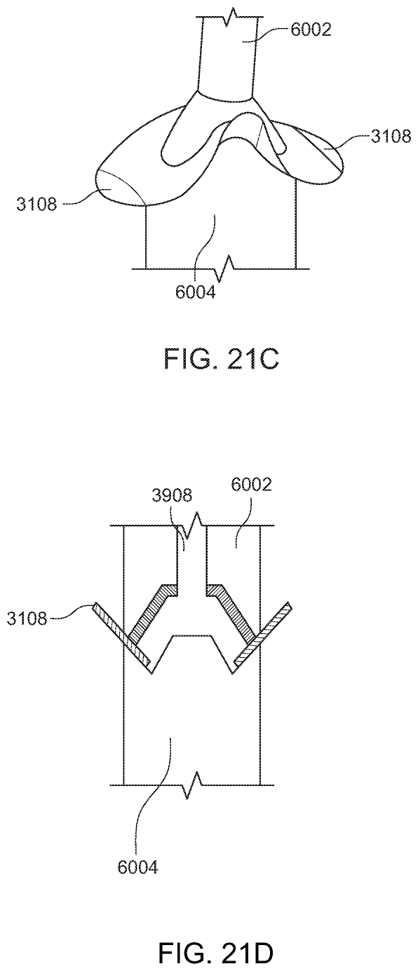

In certain forms of the technology, for example as illustrated in FIGS. 21A to 21D, the seal-forming structure 3100 includes one or more tabs 3108 for the patient 1000 or any other user to grip the seal-forming structure 3100. The tabs 3108 are regions of the seal-forming structure 3100 that do not have an adhesive surface facing towards the patient.

The tabs 3108 may be located at a periphery of the seal-forming structure 3100. For example, in the example illustrated in FIGS. 21A to 21D, the seal-forming structure 3100 comprises two tabs 3108, each located on a lateral periphery of the seal-forming structure 3100, and the two tabs 3108 are located opposite each other on either side of the opening 3110 in the seal-forming structure 3100 that allows gas to pass to the patient's nares.

The tabs 3108 may provide gripping portions for the patient 1000 to grasp the seal-forming structure 3100 and to transfer the seal-forming structure 3100 to their face without their fingers having to contact the adhesive or the adhesive surface 3102 and/or to grasp the seal-forming structure 3100 for the purposes of removing the seal-forming structure 3100 from the face. This ensures that adhesion strength of the adhesive is not lost in the process of affixing the seal-forming structure 3100 to the patient's face.

In certain forms, the tabs 3108 may facilitate the patient 1000 to pull and stretch the seal-forming structure 3100. This is particularly useful in forms of the technology using stretch-release adhesives, which are discussed in detail in subsequent paragraphs. In such forms, by stretching the adhesive surface 3102 of the seal-forming structure 3100, the adhesive strength of the stretch release adhesive may be reduced, enabling easy removal of the seal-forming structure 3100 from the patient's face.

In certain forms of the technology, the tabs 3108 may be formed as portions of non-adhesive material attached to the adhesive surface 3102. For example, the portions of non-adhesive material may be attached to an edge of the adhesive surface 3102. In other forms, the tabs 3108 may be formed as portions of non-adhesive material covering portions of the adhesive surface 3102. In other forms, the tabs 3108 may be formed as portions of seal-forming structure 3100 to which adhesive has not been applied.

### 4.3.1.8 Shape Retainers

In certain forms of the technology, the patient interface 1000 may include one or more shape retainers 3140. The shape retainer(s) may be configured to promote retention of the shape of the seal-forming structure 3100 before the seal-forming structure is made to adhere to the patient's face, for example to a sufficient extent to prevent the seal-forming structure 3100 from crumpling, folding or sagging in a way that makes it difficult for the patient 1000 to affix the seal-forming structure 3100 to their face.

The one or more shape retainers 3140 may be one or more components, an assembly or a structure which are formed with a shape and/or out of materials to provide a predetermined level of stiffness suitable to promote the desired level of retention of the shape of the seal-forming structure 3100. In certain forms, the one or more shape retainers 3140 are comprised as part of the seal-forming structure 3100. In other forms, the shape retainer(s) 3140 may be attached to the seal-forming structure 3100 to promote retention of the shape of the seal-forming structure 3100, for example by stiffening one or more regions of the seal-forming structure 3100.

### 4.3.1.8.1 Seal-Forming Structure Comprising Shape Retainers

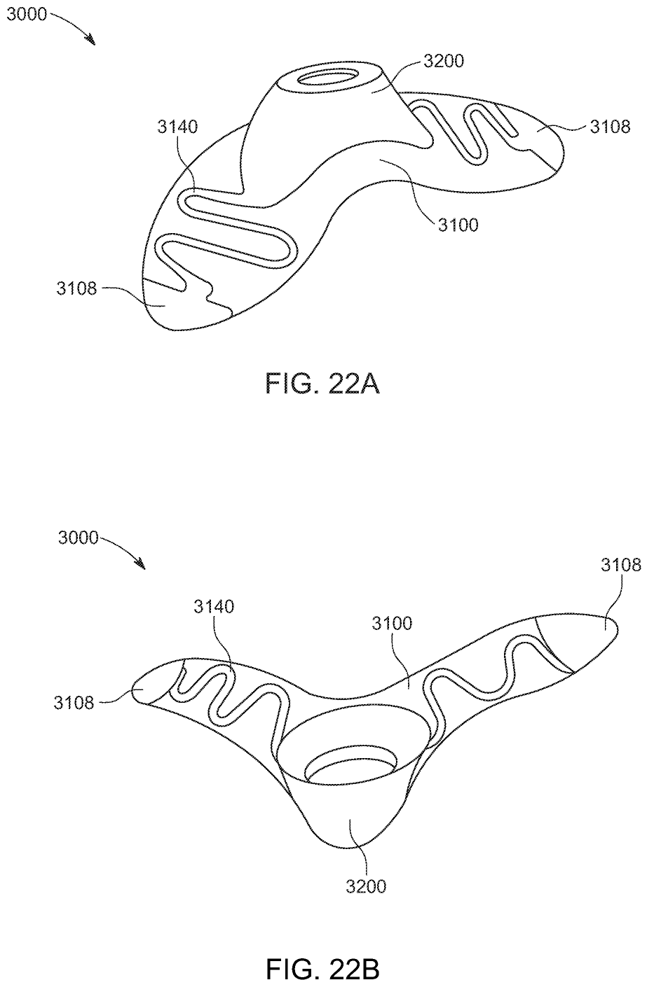

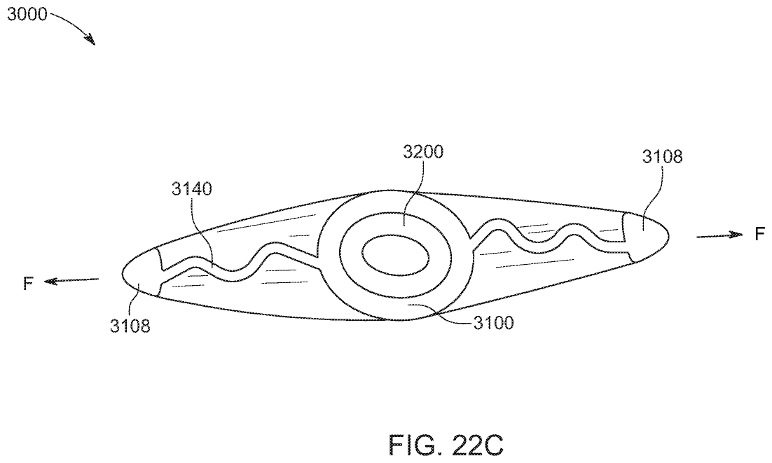

In certain forms, the seal-forming structure 3100 comprises the one or more shape retainers 3140. One such form is illustrated in FIGS. 22A to 22C, which will be described below, along with other forms of the technology in which the seal-forming structure 3100 comprises the one or more shape retainers 3140.

In certain forms, the shape retainer(s) 3140 may be provided on a side of the seal-forming structure 3100 that faces away from the patient when the patient interface 3000 is worn, for example as shown in FIG. 22A. The shape retainer 3140 may be configured to act to retain the shape of a substantial portion of the seal-forming structure 3100. For example, the shape retainer(s) 3140 may cover a substantial portion of the side of the seal-forming structure 3100 facing away from the patient.

In the illustrated exemplary form of FIGS. 22A to 22C, the shape retainers 3140 comprise elongate members extending across the front-facing surface of the seal-forming structure 3100. In this particular example, the shape retainers 3140 are sinuous members that wind across the surface of the seal-forming structure 3100 in a path that ensures any part of the seal-forming structure 3100 is sufficiently close to the shape retainers 3140 that they have the desired effect of assisting in maintaining the shape of the seal-forming structure 3100. The shape retainers 3140 thereby provide skeletal support to the seal-forming structure 3100.

In other examples, the shape retainers 3140 may have another shape, for example other configurations made up of one or more elongate members, or the shape retainers may be a shape other than elongate. In one form, the shape retainer 3140 is formed from of plurality of elongate members arranged in a lattice on the side of the seal-forming structure 3100 that faces away from the patient when the patient interface 3000 is worn. The members may be arranged with holes in the lattice in the shape of parallelograms. Other shaped lattices may also be formed in other forms of the technology, for example with triangular holes or holes of another shape(s). The holes in the lattice may not necessarily all be the same shape.

The weight and amount of shape retainer material used may not be so great as to cause discomfort to the patient 1000 when the patient interface 3000 is in use, for example through an excessive rigidity of the seal-forming structure 3100 which may make it difficult to attach the seal-forming structure 3100 securely to the patient's face, especially in the crevices of the patient's face. On the other hand, the shape retainer 3140 preferably has sufficient size and/or extent to adequately retain the shape of the seal-forming structure 3100 to assist in adhering the patient interface 3000 to the face. The amount of shape retainer material and the amount of area covered by the shape retainer material may be selected to achieve the desired balance of these considerations.

In some forms, the shape retainer 3140 comprises one or more shape retaining members, for example elongate members, arranged around a peripheral region of the seal-forming structure 3100. The central region(s) of the seal-forming structure 3100 positioned inside the peripheral members may be devoid of shape retaining members, or with few shape retaining members provided to the central region of the seal-forming structure 3100. This may provide the central region(s) of the seal-forming structure 3100 with sufficient flexibility to be able to match the contours of the patient's face while the peripheral shape retaining members provide an adequate level of overall shape retention to the seal-forming structure 3100. In some forms, one or more shape retaining members may span across the central region of the seal-forming structure 3100.

Similarly to the balance to be struck in terms of the amount of material making the shape retainers, a balance may also be found for the rigidity of the shape retainers. In certain forms, the shape retainer(s) 3140 may have sufficient flexibility that they are able to adapt to contours on the patient's face to be worn comfortably but also have sufficient rigidity that they sufficiently maintain the shape of the seal-forming structure 3100. It will be appreciated that the desired flexibility/rigidity may be obtained through appropriate selection of the hardness of the material used to form the shape retainers, and/or through appropriate selection of the material used to form them. In addition, some level of compliance in the shape retainer may be useful to avoid marking the patient's skin if the patient adopts a position where the shape retainers are pushed into their skin.

In the illustrated form of FIGS. 22A and 22B, the shape retainers 3140 are arranged to extend in a lateral direction relative to the body when worn, for example between the plenum chamber 3200 and lateral edges of the seal-forming structure 3100, where non-adhesive tabs 3108 may be located in some forms. In other forms, the shape retainer(s) 3140 may extend substantially between the plenum chamber 3200 and one or more portions of the edge of the seal-forming structure 3100 in a different direction.

In certain forms, the shape retainer 3140 may be formed together with the seal-forming structure 3100 and/or the plenum chamber 3200 as a single component. For example, in one form, the seal-forming structure 3100, which may include the shape retainer 3140, and the plenum chamber 3200 may be molded together. For example, the shape retainers 3140 may be relatively thick regions of the seal-forming structure 3100. In another example, the shape retainer 3140 and the plenum chamber 3200 may be overmolded on to the surface, for example the outer surface 3116, of the seal-forming structure 3100. Alternatively, the shape retainer 3140, and/or the plenum chamber 3200 may be laminated to the seal-forming structure 3100.

In other forms, the shape retainer(s) 3140 and the rest of the seal-forming structure 3100 and/or the plenum chamber 3200 may be components formed separately and connected together, for example by adhesion. In such forms, the shape retainer may be formed of a different material from the rest of the seal-forming structure 3100 and/or the plenum chamber 3200. Alternatively, the seal-forming structure 3100 and the shape retainer 3140 may be formed of the same material, for example they may be integrally formed.

Examples material that may be used to form the shape retainer(s) 3140 include silicone, for example low duro silicone, and TPE.

In certain forms, the shape retainer 3140 is provided to the surface of the seal-forming structure 3100 facing away from the patient in use and does not protrude outwardly from the surface of the inner surface of the seal-forming structure 3100 towards the patient. Irrespective of how the shape retainer is constructed as part of the seal-forming structure, the shape retainer 3140 may be configured to be substantially flush with the inner surface 3114. This may assist in avoid the shape retainers from marking the face when the patient interface 3000 is worn.

In one form, the shape retainer(s) 3140 may be configured such that the seal-forming structure 3100 can stretch in one direction more easily than it can stretch in a different direction. That is, the shape retainer(s) 3140 may be stress-strain anisotropic, or may provide stress-strain anisotropy to the seal-forming structure 3100. For example, the sinuous members of FIGS. 22A-22C may stretch more easily in the direction indicated by the letter F in FIG. 22C than in a perpendicular direction.

In certain forms, the shape retainer(s) 3140 may be further configured to alter the characteristic of the deformation of the seal-forming structure 3100 in different directions, for example the shape retainer(s) 3140 may be configured so that deformation of the seal-forming structure in one direction is plastic deformation and deformation of the seal-forming structure in another direction is elastic deformation. Alternatively, the transition between elastic and plastic deformation may vary for different directions.

In certain forms of the technology, the adhesive surface 3102 of the seal-forming structure 3100 may be provided with, or formed from, a stretch-release adhesive. These forms of the technology will be described in more detail later but aspects of these forms that relate to shape retainers 3140 will now be described.

In certain forms of the technology, the shape retainer(s) 3140 is configured such that a direction in which the seal-forming structure 3100 can be relatively easily stretched is aligned, or substantially aligned, with a direction in which the seal-forming structure 3100 may be stretched in order to release the stretch-release adhesive. For example, in the form of the technology shown in FIGS. 22A to 22C, the sinuous members are arranged such that the members are predominantly oriented in a direction perpendicular to the direction denoted by F in FIG. 22C, with curved portions connecting the portions oriented in this direction. This configuration means that the sinuous shape retainers wind outwardly in the direction of arrow F. in such a configuration, the shape retainer(s) 3140 may deform more easily when a force is applied in the direction F. In the case of the form shown in FIGS. 22A to 22C, this direction is in a lateral direction when the patient interface 3000 is worn. Where non-adhesive tabs 3108 are used, this direction may be in line with the direction of the non-adhesive tabs 3108 compared to the plenum chamber 3200. In contrast, the seal-forming structure 3100 is less easily stretched by applying a force in a direction perpendicular to F.

This form of technology may be useful in enabling the use of stretch-release adhesive while still benefiting from the shape retention of the shape retainers 3140.

In another form, the shape retainer(s) 3140 may be configured to delaminate or otherwise detach from the seal-forming structure 3100 when a force, F, is applied in a certain direction. For instance, the shape retainer(s) 3140 may be configured to detach from seal-forming structure 3100 when the patient 100 pulls the tab(s) 3108 to cause the seal-forming structure 3100 to detach from the patient's face by reducing the adhesive strength of a stretch-release adhesive.

In one form, silicon beads material may be used to form the shape retainer(s) 3140. Silicon beads may have a tendency to bond less strongly with the seal-forming structure 3100 than the internal bonds between the silicon beads. This may cause the shape retainer(s) 3140 to delaminate and/or detach from the seal-forming structure 3100 when a force is applied in a certain direction on the seal-forming structure 3100. In another form, the shape retainers 3140 may be formed as a TPE overmold on the rest of the seal-forming structure 3100, and this has been found to result in delamination of the shape retainer when the seal-forming structure 3140 is stretched.

By detaching from the seal-forming structure 3100, the shape retainer(s) 3140, once detached, does not add to the force required to cause the stretch-release adhesive to detach the seal-forming structure 3100. This may reduce the amount of stretching that is needed to release the stretch-release adhesive than would otherwise be the case, avoiding excessive forces to release the adhesive that may be uncomfortable to the patient 1000. 4.3.1.8.2 Front Layer Shape Retainer

As stated above, in certain forms of the technology, the shape retainer(s) 3140 may be attached to the seal-forming structure 3100 to promote retention of the shape of the seal-forming structure 3100, for example by stiffening one or more regions of the seal-forming structure 3100.

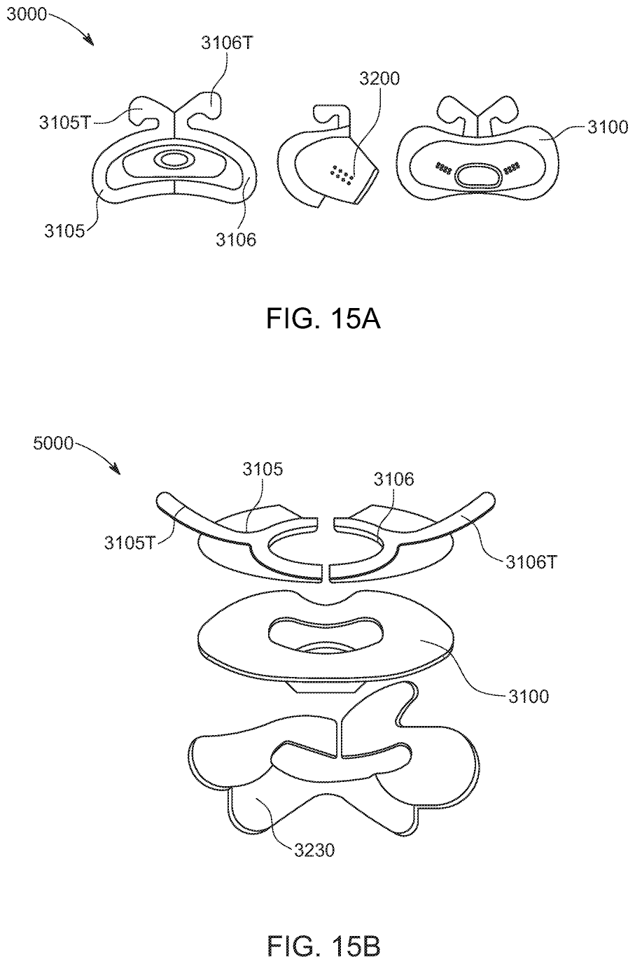

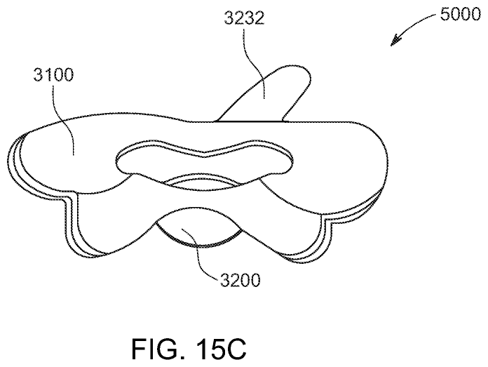

In certain forms of the technology, for example as shown in FIGS. 15B and 15C, the patient interface 3000 may further include a shape retainer 3140 in the form of a front layer 3230 which is configured to retain the shape of the seal-forming structure 3100 and thereby assist in positioning the patient interface 3000 in a therapeutically effective position on the patient's face before the front layer is removed. The front layer 3230 may be removably attached to an anterior surface of the seal-forming structure 3100, and optionally the plenum chamber 3200. For example, the front layer 3230 may be adhered to the anterior surface of the seal-forming structure 3100 and the plenum chamber 3200 through a weakly bonded adhesive such that the front layer 3230 is readily removable by the patient once the patient interface 3000 is in position. The adhesive may be provided on the surface of the front layer so that, when the front layer is removed, little or no adhesive can be felt on the anterior surface of the patient interface 3000.

In certain forms, the front layer 3230 may be configured to maintain the seal-forming structure 3100 in a certain shape prior to its removal. There are several advantages to this feature. For instance, the front layer 3230 can prevent the seal-forming structure 3100 and/or the plenum chamber 3200 from altering their shape before being affixed to the patient's face. Further, in a manufacturing process, the front layer 3230 may allow the material forming the seal-forming structure 3100 to be manipulated more easily, e.g. spooled and handled without deforming or otherwise damaging the material used to form the seal-forming structure 3100.

In one form, the front layer 3230 may be made of a material and be formed in a shape such that the front layer is relatively rigid compared to the seal-forming structure 3100 (which may be flexible, as explained above).

As explained above, the front layer 3230 may be removable from the seal-forming structure 3100 once the seal-forming structure 3100 adheres to the patient's face. The front layer 3230 may be provided with an extension 3232 (which may alternatively be referred to as a tab) which is configured to extend outside the perimeter of the seal-forming structure 3100 and/or plenum chamber 3200 when the front layer is attached thereto. The extension 3232 is useful for the patient 1000 to grip with their fingers and remove the front layer 3230 once the seal-forming structure 3100 is affixed to the face.

In certain forms, the front layer 3230 may be attached to the seal-forming structure 3100 through adhesion. In one form, the adhesive used may be applied so that the surface of the seal-forming structure 3100 which was in contact with front layer 3230 is not tacky when the seal-forming structure 3100 is separated from the front layer 3230.

In an alternative form, the front layer 3230 may remain attached to the seal-forming structure 3100 throughout the time when the patient interface 3000 is in use.

In certain forms, the front layer 3230 may be shaped to closely imitate or generally resemble the features of the patient's face in the region to which the seal-forming structure 3100 is to be attached in use. In the form illustrated in FIGS. 15B and 15C, for example, the front layer 3230 comprises two creases 3232 positioned and shaped in order to insert into the patient's alar crease when the patient interface 3000 is brought into contact with the face. When the patient pushes the front layer 3230 towards their face, the seal-forming structure 3100 is therefore pushed into the alar crease, assisting with the adhesion of the seal-forming structure 3100, which may otherwise be a region in which it is difficult to obtain adhesion because of the contours of that region. Similar features may be provided for other features of the target sealing region.

In certain forms, the shape and size of the front layer 3230 may be customized to an individual patient's face. Alternatively, the front layer 3230 may be one of a plurality of different front layers 3230 of different shapes and sizes so that the patient may select the front layer 3230 that most closely resembles their face shape and/or size. This may assist the patient in correctly positioning the patient interface 3000 on their face.

In the form of the technology shown in FIGS. 15B and 15C, the front layer 3230 has an annular shape with a hole in a central region. The hole may be configured to receive the plenum chamber 3200 with the front layer 3230 contacting the surface of the seal-forming structure 3100 facing away from the patient in use. The front layer may comprise one or more slits in order to make the front layer easier to remove.

In certain forms (not shown in the Figs.), the front layer 3230 may comprise multiple pieces. For instance, the front layer 3230 may be formed as two pieces, or preferably three pieces. The different pieces of the front layer 3230 may be changed in orientation relative to each other. In cases where the front layer 3230 is relatively rigid, this may allow the different sections of the seal-forming structure 3100 to be oriented to allow them to adhere well to the patient's face, for example to be pressed into the crevices and corners of the patient's face. In one form, the front layer 3230 may include three pieces—a central piece, a left piece and a right piece. The left piece and the right piece may be configured to lie on either side of the central piece. The central piece may have a substantially oval shape. The left piece and the right piece may correspondingly have concave shapes which are configured to mesh with the central piece.

In one form, the front layer 3230 may be made of plastic.

The front layer 3230 may be considered to be a form of applicator to assist in positioning the patient interface 3000 in a therapeutically effective position on the patient's face. In the form of the technology described in this section and illustrated in FIGS. 15B and 15C, the applicator forms part of the patient interface 3000 in the sense that it may be attached to the patient interface 3000 prior to donning the patient interface. In other forms of the technology there may be provided an applicator 3800 that is separate to the patient interface 3000, such as described in more detail below.

### 4.3.1.9 Multiple Seal-Forming Structures

In certain forms of the technology, a patient interface system 5000 may include a plenum chamber 3200 which is configured to be interchangeably attached to each of a set of multiple seal-forming structures to form patient interfaces. Each of the multiple seal-forming structures may be configured to adhere to a different facial region.

Regular use of the patient interface 3000 of the forms of the technology described involves repetitive application and removal of the seal-forming structure 3100. Every time the seal-forming structure 3100 is removed, it may remove some skin cells which get stuck to the adhesive and disengage from the skin. Over time, this removal of skin cells may cause trauma to the region of the patient's face to which the seal-forming structure 3100 adheres.

To mitigate against this problem, forms of the technology provide a patient interface system 5000 comprising multiple seal-forming structures which cover different regions of the facial skin. This allows the patient to vary which facial region is adhered to so that the same facial region is not subjected to trauma on sequential therapy sessions and the facial regions are allowed some time to recover in between.

In one form, for example as shown in FIG. 7, the patient interface system 5000 may include a first seal-forming structure 3120, a second seal-forming structure 3130 and a plenum chamber 3200. The first seal-forming structure 3120 comprises a first sealing surface which is configured to adhere to a first region of the patient's face. The second seal-forming structure 3130 comprises a second sealing surface which is configured to adhere to a second region of the patient's face. The first region may be different to the second region.

In certain forms, the second region 3130 of the patient's face may surround the first region 3120. That is, parts of the second region 3130 may be positioned radially outwardly from radially adjacent parts of the first region 3120. Further, in certain forms, there may be no overlap between the first region and the second region.

In one form, the first region may comprise, but is not limited to, one or more of the alar rim, a superior region of the lip superior (e.g. the subnasale region, which may include parts of the lip superior immediately inferior to the subnasale), and the columella.

In one form, the second region may comprise, but is not limited to, one or more of the alar, the alar crease, the lip superior, and the pronasale.

In other forms of the technology, the regions of the patient's face that the first and second seal-forming structures 3120 and 3130 are configured to adhere to may differ from those described above.

Each time there is a therapy session, the patient is able to select a seal-forming structure that is of the same type as either of the first seal-forming structure 3120 and the second seal-forming structure 3130 and connect it to the plenum chamber 3200 to form a patient interface 3000. The patient 1000 can interchange between the first seal-forming structure 3120 and the second seal-forming structure 3130 to allow the skin in each of the facial regions to recover, for example by alternating between the first and second seal-forming structure types for successive sessions. This is especially useful when respiratory therapy requires use of the patient interface 3000 every day or otherwise regularly.

### 4.3.1.10 Composition of the Seal-Forming Structure

### 4.3.1.10.1 Materials—Seal-Forming Structure

In certain forms of the present technology, the seal-forming structure 3100 is constructed from a material having one or more of the following properties: biocompatibility; soft; flexible; stretchable and optionally resilient. In exemplary forms of the technology, the seal-forming structure 3100 is formed from silicone or a thermoplastic elastomer (TPE).

TPE may be a particularly advantageous material for forming the seal-forming structure in certain forms of the technology as it is inexpensive, recyclable, soft and can be used with a variety of adhesives such as acrylate-based adhesives. Further, TPE can be molded into thin sections which can stretch and retain the stretched state. This may allow TPE-based seal-forming structures 3100 to form a very close seal with regions of the face such as alar creases. The plastic-deformability of TPE implies that a seal-forming structure 3100 which seals with a region of the patient's face does not attempt to spring back into its original shape. This, in turn, implies that there is no pulling force applied on that region, thereby reducing the discomfort caused by wearing the patient interface. This may be a particularly important consideration for sealing with sensitive areas of the patient's face, such as the alar crease region.

The ability of TPE to be cut into thin sections may allow the seal-forming structure to adhere closely to the skin surface. This has two advantages. Firstly, the thin sections allow highly close conformation to the underlying skin and facial features. Secondly, a thinner section may be more likely to stay flush with the skin surface and not to protrude from it. This may reduce the likelihood of the seal-forming structure 3100 coming undone, either partially or completely, due to friction between the patient's face and surrounding objects such as pillowcases, counterpanes, etc.

An advantage of forming the seal-forming structure 3100 from a soft material (such as TPE) and in a configuration which enables the seal-forming structure 3100 to flex is that the seal-forming structure 3100 is able to conform to the shape of a patient's face and to continue to do so as the shape of the patient's face changes, for example if they change position. It has been explained above why this is advantageous.

In certain forms it may be advantageous to use a material for the seal-forming structure 3100 that undergoes plastic deformation as it flexes and/or stretches, i.e., it does not spring back to its original shape or dimensions, or at least does not entirely spring back. This prevents the seal-forming structure 3100 imparting shear forces on the patient's skin as it attempts to revert to its original shape/size.

In certain forms, the material used to form the seal-forming structure 3100 may be stretchable. The material may be elastically deformable or plastically deformable when stretched. The ability to stretch may be useful for easy removal of the seal-forming structure 3100 from the patient's face when a stretch-release adhesive is used. These adhesives are discussed in further detail in the next section. An example of a stretchable material that may be suitable for use with a stretch-release adhesive is silicone rubber.

Examples of materials that may be suitable for use as a seal-forming structure in certain forms of the technology are the 3M™ Nexcare™ tape and Leukoplast tape. These tapes also have an adhesive surface. In some forms, the material may comprise a rayon substrate to which an adhesive is applied. In one form, the seal-forming structure may be formed from, or may comprise, 3M™ Product No. 2484, 3M™ Medical tape 9833, or a similar type of product or a product having a similar structure. Multiple layers of a tape or product may be used to form the seal-forming structure 3100.

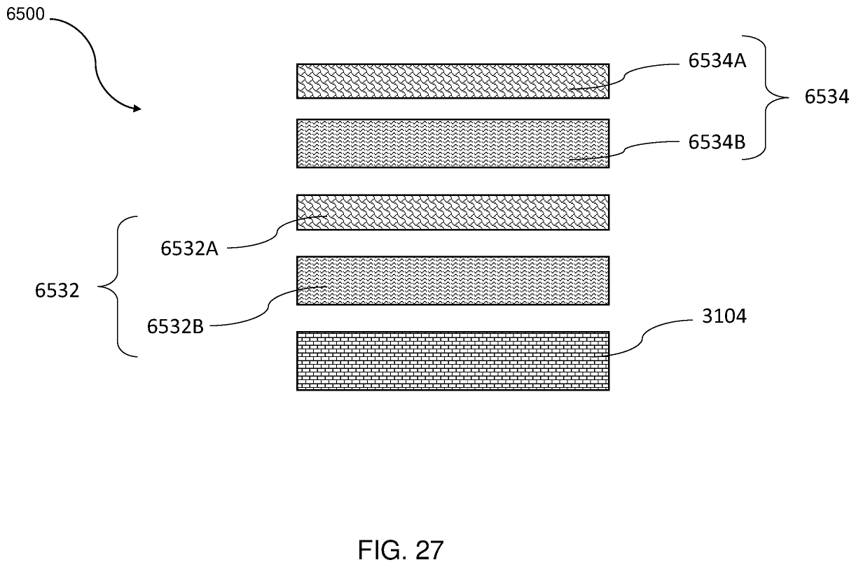

FIG. 27 illustrates an exploded sectional view of a part of an adhesive seal-forming structure 3100 according to one form of the technology. The seal-forming structure 3100 comprises a plurality of layers: a first layer assembly 6532, a second layer assembly 6534 and a removable layer 3104. While, in the form shown in FIG. 27, the seal-forming structure 3100 comprises two layer assemblies 6532 and 6534, in other forms, the seal-forming structure 3100 may comprise only one layer assembly. In other forms, the seal-forming structure 3100 may comprise three or more layer assemblies.

The first layer assembly 6532 comprises a backing layer 6532A and an adhesive layer 6532B. The second layer assembly comprises a backing layer 6534A and an adhesive layer 6534B. If there are additional layer assemblies in other forms, each of the additional layer assemblies may also comprise a backing layer and an adhesive layer.

As shown in FIG. 27, the layers may be arranged such that a backing layer, for example the second backing layer 6534A, forms one side of the seal-forming structure 3100 and the removable layer 3104 forms the opposite side of the seal-forming structure 3100. The adhesive layer 6534B lies sandwiched between the second backing layer 6534A and the first backing layer 6532A. The first adhesive layer 6532B lies sandwiched between the first backing layer 6532A and the removable layer 3104.

The removable layer 3104 acts to protect the second adhesive layer 6532B until the seal-forming structure 3100 is attached to the patient's face. The function and properties of exemplary removable layers 3104 are explained in greater detail in subsequent portions of this specification. The removable layer 3104 may alternatively be referred to as a liner.

In certain forms, each backing layer may be formed from a thin layer of plastic, for example a polyurethane film. Each adhesive layer may be formed from a suitable adhesive, for example a medical silicone adhesive. In certain forms, each adhesive layer may be formed from a Hi-Tack 3M™ Medical Silicon Adhesive. The removable layer 3104 may be formed from a thin plastic or polymer, for example a polypropylene film. In one form, the removable layer 3104 is formed from a polypropylene film with a non-silicone release on one side. In one exemplary form of the technology, the seal-forming structure 3100 is formed from two layers of the 3M™ Product No. 2484.

In some forms, the patient interface 3000 may be formed with the plenum chamber 3200 attached to outer backing layer 6534A of the seal-forming structure 3100.

### 4.3.1.10.2 Thickness—Seal-Forming Structure

If the seal-forming structure 3100 is too thin, then it may make it difficult for the patient interface 3000 to be removed and re-applied to the patient's face in case the patient interface 3000 is applied to an incorrect region of the patient's face. A seal-forming structure 3100 which is too thin tends to easily lose its shape and can fold onto itself when removed, getting stuck to itself and difficult to manipulate. A seal-forming structure 3100 that is too thin may also tear easily.

On the other hand, if the seal-forming structure 3100 is too thick, then the seal-forming structure 3100 may tend to feel quite stiff against the patient's face which may cause discomfort in use. Further, a stiff and/or thick seal-forming structure 3100 may be difficult to press into the corners and/or crevices of a patient's face, thereby impacting on the quality of seal against the face that can be achieved. The thickness of the seal-forming structure 3100 may also impede the seal-forming structure from altering its shape to accommodate facial movement of the underlying facial regions.

An appropriate thickness will depend on the selection of material used for the seal-forming structure 3100, and also on the shape and structure of the seal-forming structure.

In certain forms, the seal-forming structure 3100 may have a thickness which is substantially in the range of approximately 0.2 mm to 0.3 mm, excluding the thickness of the removable layer 3104, or around 0.3 mm to 0.45 mm including the removable layer. For example, in the case of the seal-forming structure 3100 of FIG. 27, where two layers of 3M™ Product No. 2484 are used, the thickness may be substantially 0.24 mm without the removable layer 3104, and 0.33 mm with it. In this exemplary form, the backing layer 6532A and the backing layer 6534A may each have a thickness of approximately 0.02 mm, and the adhesive layer 6532 B and adhesive layer 6534B may each have a thickness of approximately 0.1 mm. In other forms, the seal-forming structure 3100 may have a different thickness. The suitable thickness to provide a suitable balance of shape retention and comfort may depend on several factors, including for example the selection of materials used and the number of layers.

### 4.3.1.10.3 Other Characteristics—Seal-Forming Structure

In certain forms, it may be advantageous for the seal-forming structure 3100 to be able to transmit moisture through it. This may enable moisture from the patient's skin to pass through the seal-forming structure 3100. As the patient interface 3000 may typically be worn for long periods, this may be desirable to avoid moisture build up under the seal-forming structure 3100 and discomfort. In certain forms, the material used to form the seal-forming structure 3100 may have a moisture vapour transmission rate (MVTR) in a range of substantially 50 gm/m²/day to 1000 gm/m²/day, for example substantially 700 gm/m²/day in the case of the 3M™ Product No. 2484. Higher values of MVTR may be more desirable when the patient interface 3000 is to be worn for extended periods of time. When the patient interface 3000 is designed for use over relatively shorter periods of time, the seal-forming structure 3100 may have lower values of MVTR.

It has already been described that it may be advantageous for the seal-forming structure 3100 to be formed from a deformable material.

In some forms, it may be desirable for the seal-forming structure 3100 to be transparent or semi-transparent. This may reduce the visual footprint of the patient interface 3000 on the patient's face and help make the patient interface more desirable to wear for extended periods of time.

### 4.3.1.11 Forms of Adhesive

Forms of the technology provide a patient interface 3000 comprising a seal-forming structure 3100 that comprises at least one adhesive surface configured in use to adhere to a region of the patient's face surrounding the entrance to the patient's airways to form a seal.

Any suitable adhesive may be used, and in the following paragraphs is described suitable properties of an adhesive used in certain forms of the technology. It will be appreciated that, unless expressly indicated otherwise, forms of the technology are not limited to certain adhesives. Furthermore, in some forms, the adhesive may be comprised of one or more constituent adhesive materials.

In certain forms the adhesive applied to the adhesive surface of the seal-forming structure 3100 is an adhesive that adheres to skin with an adhesion strength that is able to maintain the adhesion when forces of the direction and magnitude that are typically experiences when using a patient interface 3000 are imparted so that the seal-forming structure 3100 does not become too easily disengaged during normal use. Similarly, the adhesion strength should not be so great that the patient interface 3000 cannot be removed following the cessation of therapy without causing trauma to the skin. In certain forms, the seal-forming structure may have an adhesion strength substantially in the range of approximately 2 N to 3.5 N per 25.4 mm width.

In certain forms, the adhesive may be configured to maintain the desired level of adhesion with the facial skin despite the presence of moisture (for example, sweat) on the patient's skin and/or heat from the skin.

It is desirable for the adhesive to be able to adhere to the skin irrespective of the contours of the patient's skin to which it is adhering, e.g. whether there are creases or crests or flat regions.

It is desirable for the adhesive to be odour-less (as far as can be typically perceived by a patient) and to be colourless or to have an aesthetically appealing colour. Furthermore, certain forms of the technology use adhesives in which no or little residue is left behind following removal from the skin. Adhesives not having these characteristics may be used in some forms of the technology and may operate effectively, but may be undesirable to patients.

Some adhesives require certain steps to be taken to prepare the surface to which the adhesive is to adhere in order to form an effective bond. For example, some adhesives require a patient's skin to be swabbed with a cleaning fluid, such as alcohol, prior to use. The need for such preparation may be undesirable as it requires extra steps from a patient and they may not prepare the skin effectively, particularly when tired. Therefore certain forms of the technology use adhesives that do not require such surface preparation.

It is desirable to use an adhesive that is effective at adhering to skin but does not adhere well to hair. This avoids the patient experiencing discomfort when the seal-forming structure is removed and hair is removed along with it.

It is desirable for the adhesive to be suitable for the seal-forming structure 3100 to be adhered to the skin and, if necessary, to be removed and then re-adhered to the patient's skin. A patient may not always correctly locate the seal-forming structure 3100 when first donning the patient interface 3000. For example, it may be located in an uncomfortable or otherwise undesirable position. In certain forms of the technology, the adhesive surface of the seal-forming structure 3100 is provided with an adhesive that is suitable for at least one re-application of the seal-forming structure 3100 on the patient's face.

The adhesives used on the 3M™ Product No. 2484, the 3M™ Nexcare™ tape and Leukoplast tape are examples of suitable materials carrying adhesives that possess one or more of the above characteristics and are used in certain forms of the technology. For example, a rubber zinc oxide adhesive may be used. In other forms, other adhesive tapes are used, for example acrylic or acrylate adhesives, or silicone adhesives. The adhesives on adhesive tapes are already provided on a substrate (i.e. the tape), which may be advantageously used as the seal-forming structure 3100, or part thereof, or may be readily attached to the seal-forming structure 3100. In the case of the 3M™ Product No. 2484, the adhesive is a silicone adhesive (“Hi-Tack 3M medical silicone adhesive”). The 3M™ Product No. 2484 has an adhesion strength of 2.8 N per 25.4 mm width. In the case of the 3M™ Medical tape 9833, the adhesive is an acrylic/acrylate adhesive.

In other forms of the technology, the adhesive for the seal-forming structure 3100 may be deposited directly on the adhesive surface 3102, for example as described below. The adhesive may be deposited on the adhesive surface as part of the manufacturing process. Alternatively, in some forms, the patient interface 3000 may be supplied in a form where the adhesive is not yet applied to the adhesive surface and the patient (or clinician) applies the adhesive to the surface prior to use.

### 4.3.1.11.1 Fluid Adhesive

In certain forms of the present technology, the patient interface 3000 comprises a seal-forming structure 3100 having at least one surface to which a fluid adhesive may be applied to form an adhesive surface.

### 4.3.1.11.2 Spray Adhesive

In alternative forms of the present technology, the patient interface 3000 comprises a seal-forming structure 3100 having at least one surface to which a sprayable adhesive may be sprayed to form an adhesive surface.

### 4.3.1.11.3 Action Release Adhesive

In some forms, the seal-forming structure 3100 may be attached to a patient's face using one or more action-release adhesives. The action-release adhesive(s) may be configured so that its adhesive strength is reduced when an “action” is effected. The reduction in the adhesion strength may be sufficient to allow the seal-forming structure 3100 to be easily removed from the patient's face while causing an acceptable level of discomfort. Examples of actions are provided below. In some forms, the action may be some change or effect that is applied to the adhesive, or to a component (such as the seal-forming structure 3100) to which the adhesive is applied.

In certain forms of the present technology, the patient interface 3000 comprises a seal-forming structure 3100 having an adhesive surface 3102 where the adhesive strength of the adhesive surface 3102 can be reduced by the deformation of the adhesive surface 3102. The adhesive surface 3102 may, when deformed, be configured to lose or alter its adhesive properties substantially or wholly, so as to reduce or lose its adhesion strength. This allows the patient 1000 to easily remove the seal-forming structure 3100 by deforming it.

In certain forms, the adhesive surface 3102 may be provided with, or formed from, a stretch-release adhesive. A stretch-release adhesive may have certain adhesive properties only when the adhesive surface 3102 is substantially unstretched. The adhesive surface 3102 may be configured to have reduced and/or no adhesive properties when the adhesive surface 3102 is stretched. In certain forms, acrylate-based adhesives may be used as a stretch-release adhesive for adhesive surface 3102, for example Fixomull™ or 3M™ Stretch Release Tape.

In certain forms, the adhesive surface 3102 may be formed from a carrier material to which the stretch-release adhesive may be applied. In some examples, the carrier material may be polyurethane. Polyurethane may be suitable as it is light, pliable, ductile, safe to use on skin, breathable, can carry an adhesive, and other components of the patient interface may be overmoulded to it (such as explained elsewhere in this specification). In other forms, other suitable carrier materials may be used.

In certain forms, an adhesive surface 3102 provided with a stretch-release adhesive may comprise a polyurethane removable layer 3104, for example a stretchable polyurethane layer. A layer that is removable from an adhesive layer to enable the adhesive to be adhered to the patient may be referred to as a liner in this specification. The use of a stretch-release adhesive may enable easy removal of the seal-forming structure 3100 from the patient's face by stretching the adhesive surface 3102.

In other examples, the action-release adhesive(s) may include heat-release adhesives which lose adhesion strength as they are heated, or when exposed to a temperature greater than a pre-determined level. In another example, the action-release adhesive may be a water-release adhesive that causes the seal-forming structure 3100 to detach when the adhesive (and/or the seal-forming structure 3100 carrying the adhesive) is made wet and/or moist. In yet another example, the action-release adhesive(s) may be configured to lose adhesion when exposed to radiation of a certain frequency, such as ultraviolet light.

4.3.1.11.4 Adhesive tape sections

In alternative forms of the technology, the patient interface 3000 comprises one or more adhesive tape sections configured to be provided to the at least one sealing surface of the at least one seal-forming structure. Each adhesive tape section may include a first adhesive on one side and a second adhesive on the other side. The first adhesive may be configured to secure the adhesive tape section to the seal-forming structure 3100. The second adhesive may be configured to secure the seal-forming structure 3100 against the skin of the patient 1000. Therefore, the second side of the adhesive tape sections provides the adhesive surface.

The adhesive tape section may be provided with a removable layer(s) to protect the first and second adhesives from contamination and/or loss of adhesion before the seal-forming structure 3100 is to be assembled.

In some forms (not shown in any of the Figs.), the adhesive tape section may be provided with one or more shape retainers which may improve shape retention of the adhesive tape section. The shape retainers may be provided only to the adhesive tape section but not to the removable layer(s).

The shape retainers provide structural support to the adhesive tape section when the removable layer(s) is/are removed, thereby preventing the adhesive tape section from crumpling or substantially altering shape or orientation. If the adhesive tape section's shape and/or orientation is sufficiently altered, it may lead to several disadvantages.

Firstly, the seal-forming structure 3100 may adhere to incorrect locations on the patient's face. For instance, if a seal-forming structure 3100 is designed to attach to the alar rim region of the nose, then an altered shape of the adhesive surface may cause other regions of the nose or face that are outside the alar rim region such as the septum 3132, columella, or the nostril region to come in contact with the adhesive surface. This may cause poor sealing, inconvenience, discomfort and/or pain to the patient 1000.

Secondly, if the adhesive tape section crumples or deforms when the removable layer(s) is/are released, one or more portions of the adhesive tape section may fold onto each other. This may result in the surface area of exposed adhesive being reduced, which may lead to ineffective sealing between the patient's face and the seal-forming structure 3100.

Thirdly, if one or more portions of the adhesive tape section fold onto each other, it may lead to an uneven adhesive surface 3102 which is likely to leave marks on a patient's face.

The shape retainers of the adhesive tape section may have the same properties, configuration, and/or advantages as those of shape retainer(s) 3140 described earlier in this specification. Further, the shape retainers of the adhesive tape section may be formed in the same manner as that of the shape retainer(s) 3140.

### 4.3.1.11.5 Removable Layer

In certain forms, the patient interface 3000 may further include a removable layer 3104 which is removably attached to, and covers, the adhesive surface 3102. The removable layer 3104 may be removed prior to the patient interface 3000 being positioned in sealing contact with the patient's face. As stated above, the removable layer 3104 may be referred to as a liner. The removable layer 3104 may be made of any appropriate material, including paper, such as wax paper, or plastic, such as polypropylene.

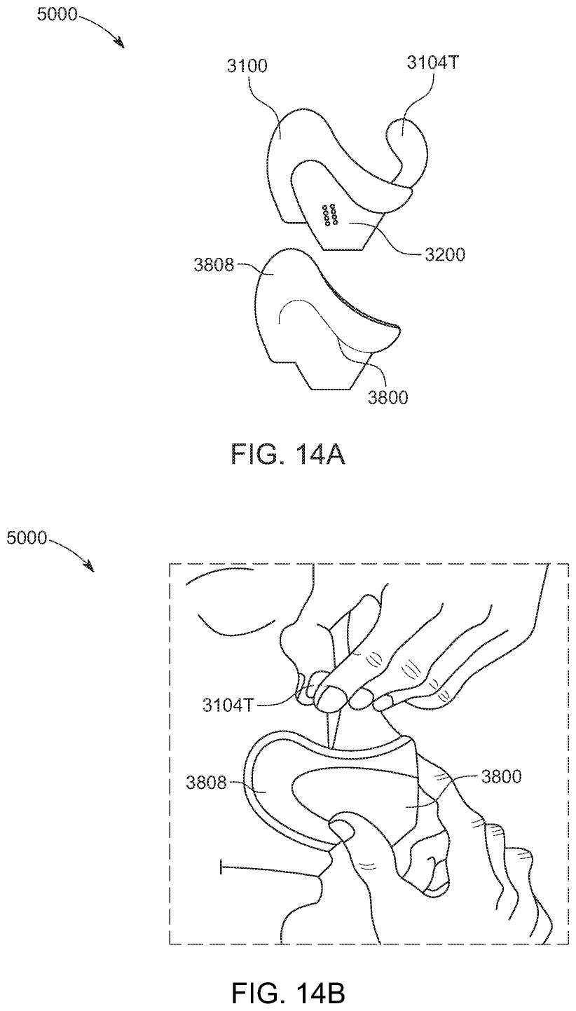

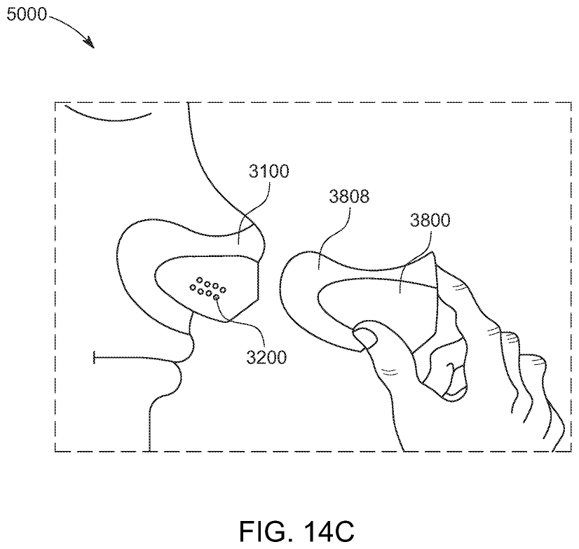

The removable layer 3104 may serve to protect the adhesive on the adhesive surface 3102 from contamination, unintentionally being adhered to other surfaces and/or loss of adhesion when the patient interface 3000 is not in use. The removable layer 3104 may be attached to the adhesive surface 3102 by the adhesive when the patient interface 3000 is transported, stored, or otherwise not in use, but may be configured to form a generally weak attachment with the adhesive surface 3102, for example by having a smooth surface that contacts that adhesive surface 3102. The removable layer 3104 may be removed by the patient 1000 before donning the patient interface 3000. Exemplary forms of the technology in which the patient interface 3000 comprises a removable layer 3104 are shown in FIGS. 14A, 14B, 15A, 15B and 17. In one form, the removable layer 3104 may include a tab 3104T which extends outside the footprint and/or perimeter of the seal-forming structure 3100. The removable layer 3104 may be peeled away by the patient 1000 by gripping the tab 3104T, as shown in FIG. 14B, and pulling the removable layer 3104 off the seal-forming structure 3100.

In one form, the removable layer 3104 comprises two parts—a first part 3105, and a second part 3106, as shown in FIGS. 15A and 15B. The first part 3105 of the removable layer 3104 may be configured to be peeled towards a first side of the patient's face, and the second part 3106 of the removable layer 3104 may be configured to be peeled towards a second side (which may be opposite to the first side). The first part 3105 may have a first tab 3105T, and the second part 3106 may have a second tab 3106T. When the first and second parts 3105 and 3106 are attached to the adhesive surface 3102 the first and second tabs 3105T and 3106T may extend in different, for example opposite, directions. The first part 3105 may be peeled away by the patient 1000 by gripping the first tab 3105T and pulling in the relevant direction. Similarly, the second part 3016 may be peeled away by the patient 1000 by gripping the second tab 3106T and pulling in the relevant direction.

The tabs 3104T, 3105T, and 3106T allow the respective removable layers 3104, 3105, 3106 to be peeled away after the patient interface 3000 is positioned correctly against the patient's face. The ability to position the patient interface 3000 against the face without the adhesive attaching (i.e. when the removable layer is in place) allows the patient to try different positions of the patient interface 3000 before it is adhered. This enables the patient to identify a comfortable and effective position for the seal-forming structure 3100 to be affixed to the patient's face. This is particularly important when the seal-forming structure 3100 is shaped to match/resemble specific regions of the patient's face to which the seal-forming structure 3100 adheres.

In a first step, the patient interface 3000, with the removable layers 3104, 3105, 3106, is placed against the patient's face until a suitable position is identified. In a second step, the patient 1000 may slip a finger beneath the tab 3104T, 3105T or 3106T and/or move the patient interface 3000 slightly forward to provide the clearance for the patient 1000 to grip the tab 3104T, 3105T or 3106T. In a third step, the tab 3104T, 3105T or 3106T is peeled away to expose the adhesive surface 3102 underneath. In the fourth step, the seal-forming structure 3100 is affixed to the patient's face by pressing the adhesive surface against the patient's face.

In one form, the tab 3104T, 3105T or 3106T may be configured to lie away from the patient's facial features when the patient interface 3000 is in the intended position. For instance, if the patient interface 3000 is configured to be attached in or around the nasal region, the tab 3104T, 3105T or 3106T may be positioned away from the nose such that the patient can grip the tab 3104T, 3105T or 3106T without the nose being in the way.

Alternatively, when the patient interface is in the intended position the tab 3104T, 3105T or 3106T may be configured to lie against a soft tissue which can deform while the finger is slipped under the tab 3104T, 3105T or 3106T for gripping. For instance, if the patient interface 3000 is configured to be attached in or around the mouth region, the tab 3104T, 3105T or 3106T may be positioned to lie against the cheek which can deform slightly to accommodate the patient's finger when gripping the tab 3104T, 3105T or 3106T.

### 4.3.2 Plenum Chamber

The plenum chamber 3200 of certain forms of the technology is configured to receive the flow of breathable gas at the therapeutic pressure for breathing by the patient from the air circuit 4170. The plenum chamber may be formed to be pressurisable to a therapeutic pressure of at least 6 cmH₂O above ambient air pressure, and up to pressures of around 20 cmH₂O or 30 cmH₂O in certain forms.

In one form, the plenum chamber 3200 has a perimeter that is shaped to be complementary to the surface contour of the face of an average person in the region where a seal will form in use. The complementary shape of the perimeter of the plenum chamber 3200 may be configured to facilitate correct positioning of the patient interface 3000 against the patient's face in use.

Alternatively, in certain forms, the plenum chamber 3200 may be shaped in a customised way to an individual patient. Alternatively, the plenum chamber 3200 of a patient interface 3000 may be selected from one of a plurality of possible forms of plenum chamber 3200, with the appropriate plenum chamber for an individual patient being selected as being most suitable for them.

In use, a marginal edge of the plenum chamber 3200 is positioned in close proximity to an adjacent surface of the face. Actual contact with the face may be provided by the seal-forming structure 3100. The seal-forming structure 3100 may extend in use about the entire perimeter of the plenum chamber 3200.

In some forms, the plenum chamber 3200 and the seal-forming structure 3100 are formed from a single homogeneous piece of material, for example silicone or TPE.

In certain forms of the technology, such as shown in FIGS. 4A to 4C, 5A, 8, 9A to 9D, 10B, 26 and 28 to 32, the plenum chamber 3200 may be substantially frustum-shaped. The plenum chamber 3200 in these forms has a first end proximal the patient in use and a second end distal from the patient in use, wherein the area of the opening at the first end is greater than that of the second end. In some forms, the plenum chamber 3200 may taper from the first end to the second end, or some portion of the plenum chamber 3200 may taper in this direction, for example a region of the plenum chamber 3200 around the plenum chamber inlet port 3202 may be tapered. The cross-section of the plenum chamber 3200 at the first and/or second ends may be substantially circular. Alternatively, the cross-section may be another shape, for example oval.

The first end and second end of the plenum chamber 3200 may be substantially open. The first end may be configured to be fluidly connected with the seal-forming structure 3100 to provide pressurised gas to the patient's airways. The second end may be substantially opposite to the opening 3110 and may form a plenum chamber inlet port 3202, as described further below.

The first end of the plenum chamber 3200 may have an area wide enough to cover the airways (e.g. span the nose or cover the nose and mouth) of the patient, thereby allowing air flow to and from the nostrils to be channelised through the patient interface 3000.

The second end of the plenum chamber 3200 may be configured to connect to the air circuit 4170 and may therefore have a smaller area in order to connect to the air circuit 4170. This facilitates a substantially air-tight connection between the air circuit 4170 and the plenum chamber inlet port 3202.

In certain forms, the walls of the plenum chamber 3200 may form a conical surface or they may be curved away from a true conical surface, for example bulging outwardly.

In an alternative form, the plenum chamber 3200 may have a substantially semi-spherical or other cup-like shape.

### 4.3.2.1 Plenum Chamber Alignment

It has already been described that a plenum chamber 3200 having a perimeter that is shaped to be complementary to the surface contour of the face of an average person in the region where a seal will form in use may facilitate correct positioning of the patient interface 3000 against the patient's face in use.

Alternatively, or in addition, a positioning guide member may be used to facilitate correct positioning of the patient interface 3000 against the patient's face when donning, which may enable the patient interface 3000 to be positioned correctly before the seal-forming structure 3100 adheres to the patient's face.

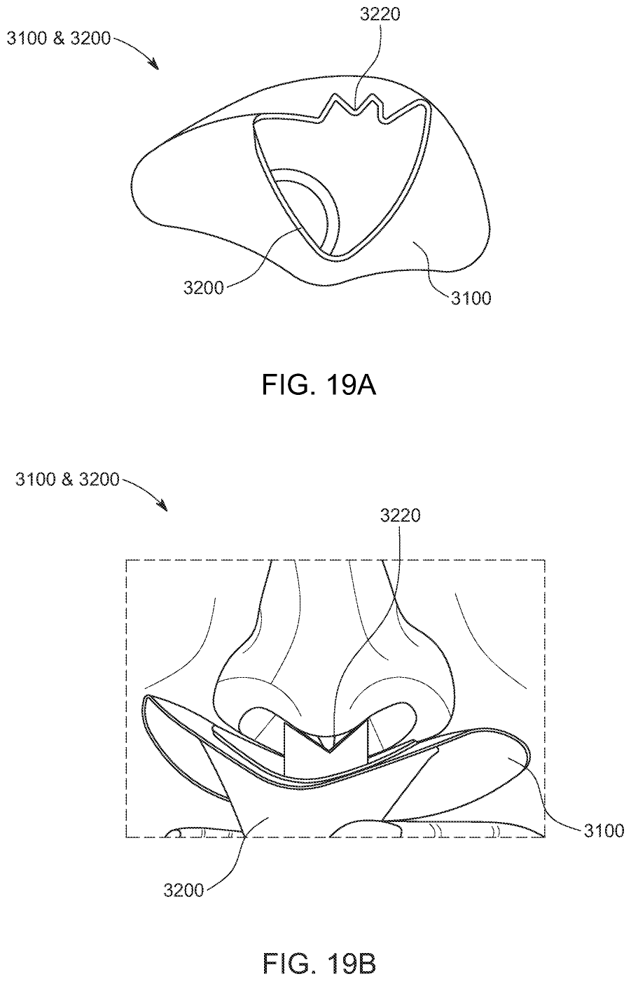

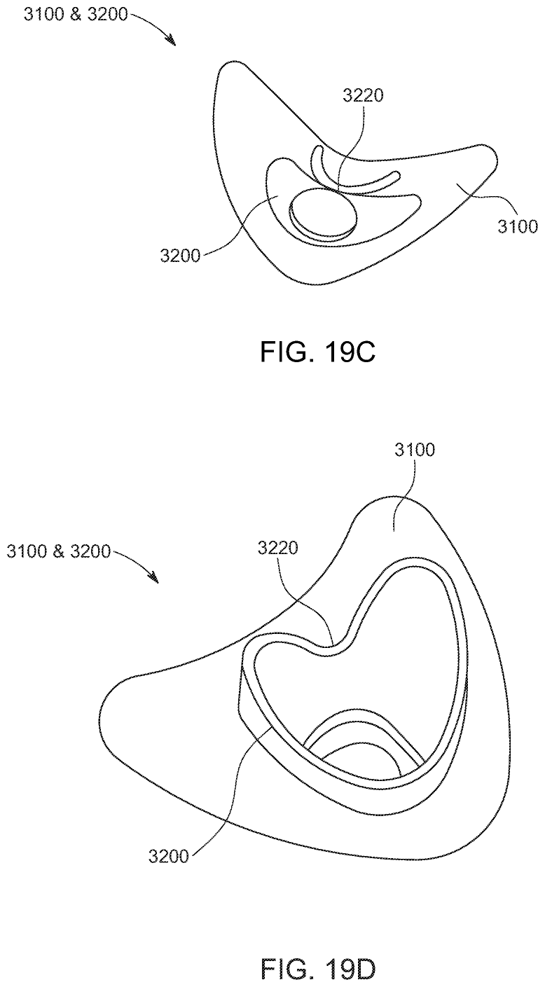

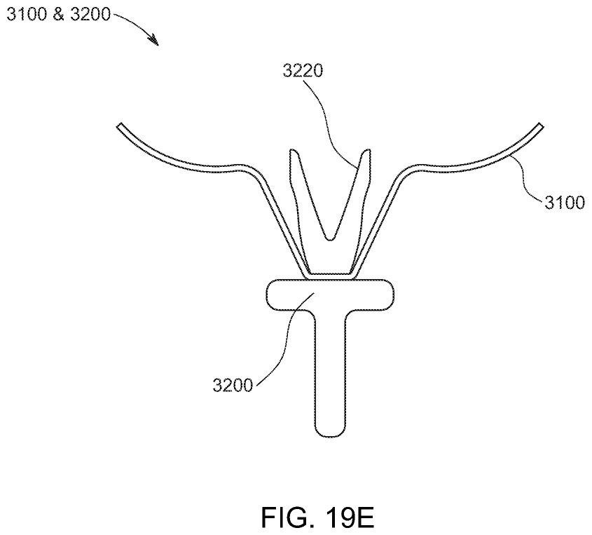

In certain forms, as shown in FIGS. 19A to 19E, the positioning guide member 3220 extends from an inner part of the plenum chamber 3200 towards the patient. The positioning guide member 3220 may be advantageous as it allows the patient 1000 to correctly position the patient interface 3000, and in some forms without the need to look at themselves to do so. This may be useful when positioning the patient interface 3000 in the dark or in the absence of a mirror or a device with a front camera.

Further, the positioning guide member 3220 may allow the patient 1000 to relocate and/or adjust the patient interface 3000 before the adhesive surface 3102 comes in contact with the patient's skin. If the adhesive surface 3102 contacts the patient's face before the patient 1000 is satisfied with the position of the patient interface 3000 against their face, there may be contamination and/or a loss in adhesion force when the patient 1000 removes the patient interface 3000 and relocates it. In addition, repeated application and removal of the seal-forming structure 1000 may inadvertently lead to skin trauma and/or ripping off of skin cells.

In certain forms, as shown in FIGS. 19A to 19E, the positioning guide member 3220 may comprise a member having a concave portion facing towards the patient's face. The concave portion may be configured to receive a portion of the patient's face when the patient interface 3000 is donned in order to assist the patient to correctly position the patient interface 3000 on their face.

In certain forms, as shown in FIGS. 19A, 19B and 19E, the positioning guide member 3220 may comprise a V-shaped member that forms part of, or protrudes outwardly from, the plenum chamber 3200. In use, the V-shaped member may be configured to receive the septum of the patient 1000 in contact with the inner apex of the V-shape. The V-shaped member may be formed from a soft material such that it can deform and not cause discomfort to the patient 1000 when the patient interface 3000 is worn. The V-shape of the V-shaped member may be rounded. The positioning guide member 3200 may also comprise a U-shaped member for a similar purpose.

In another form, as shown in FIGS. 19C and 19D, the positioning guide member 3220 comprises a member having a concave portion that has smoothly curved sides along the edge containing the concavity.

The dimensions of the positioning guide member 3220 may be such that it does not occlude the patient's airways in use. The positioning guide member 3220 may be made of a soft material, such as TPE.

In the forms of the technology shown in FIGS. 19A to 19D, the positioning guide members 3220 form part of the plenum chamber 3200, while in other forms, the positioning guide members 3220 are provided to a part of the plenum chamber 3200.

In certain forms of the technology, the positioning guide member 3220 may be removable from the patient interface 3000 after the patient interface 3000 has been donned and appropriately positioned. In some forms, it may be necessary to remove the positioning guide member 3220 before the patient interface 3000 is connected to the air circuit 4170 to ensure unimpeded flow of the supply of breathable gas.

In some forms of the technology, the positioning guide member 3220 may comprise an elongate body and a patient-contacting end. The patient-contacting end may be concave, e.g. V-shaped or U-shaped, to engage a portion of the patient's face, e.g. the septum, as explained above. The elongate body may be configured to extend through the plenum chamber inlet port 3202 with the patient-contacting end extending through the first end of the plenum chamber 3200 (the end proximal the patient in use). The positioning guide member 3220 is in this position when the patient interface 3000 is donned and, once the patient is happy with the positioning of the patient interface 3000, the positioning guide member 3220 may be removed through the plenum chamber inlet port 3202.

In certain forms of technology, prior to removal, the positioning guide member 3220 may be connected to the plenum chamber 3200 in a way that maintains the position of the positioning guide member 3220 relative to the plenum chamber 3200 prior to use and during donning of the patient interface 3000, but allows easy removal of the positioning guide member 3220 after the patient interface 3000 is appropriate positioned. For example, in one form, the positioning guide member 3220 may be lightly adhered to the plenum chamber 3200. In another form, the positioning guide member 3220 may be configured to be magnetically attached to the plenum chamber 3200. For example, the body of the positioning guide member 3220 may have a magnetic member which may magnetically attach to another magnetic member of the plenum chamber 3200.

It will be appreciated that the force necessary to be applied by the patient 1000 to remove the positioning guide member 3220 should preferably be of a magnitude that is substantially insufficient to disengage the adhesive attachment of the seal-forming structure 3100 to the patient's face.

In certain forms of the technology, the magnetic member of the plenum chamber 3200 may be configured to magnetically connect to the air circuit 4170 once the positioning guide member 3220 has been removed. For example, the magnetic member of the plenum chamber 3200 may be a magnetic member 3510 as described in detail further below.

In the illustrated forms of the technology, the positioning guide member 3220 may be located inferior to the opening in the first end of the plenum chamber 3200 (i.e. the end of the plenum chamber 3200 that connects to the seal-forming structure 3100 and is proximal to the patient in use when the patient interface 3000 is being worn).

For example, the positioning guide member 3220 may be comprised as part of, or may be provided, detachably or undetachably, to a rim around an opening in the plenum chamber 3200 through which the flow of gas is conveyed to the patient's nares. The positioning guide member 3220 may be comprised as part of, or may be provided to, a part of the rim, for example a part of the rim on an inferior side of the opening when the patient interface 3000 is being worn. Alternatively, the positioning guide member 3220 may form a substantial part of the rim of the plenum chamber 3200 around said opening. The rim may be configured to interface with one or more of the subnasale, columella, septum 3132 and/or the alar crest regions of the nose, and/or the lip superior.

In certain forms of the technology, as shown in FIG. 19E, the positioning guide member 3220 (which may be V-shaped or U-shaped) may comprise prongs that are configured to splay open when the patient interface 3000 is donned and the positioning guide member 3220 engages with the patient's septum 3132. For example, the prongs may be sufficiently long to have this flexibility, and/or the material used to form the positioning guide member 3220 may be sufficiently flexible. In the illustrated form, the prongs may extend substantially into the nostrils of the patient 1000. In forms of the technology in which the positioning guide member 3220 is configured to be removed once the patient interface 3000 is donned, the prongs come together as the positioning guide member 3220 is removed through the plenum chamber inlet port 3202, allowing the positioning guide member 3220 to be removed.

In certain forms of the technology, for example as shown in FIG. 19B, the adhesive surface 3102 of the seal-forming structure 3100 may be configured in such a way that, before application of the seal-forming structure 3100 to the face, the adhesive surface is positioned so that it does not come into contact with the face before the positioning guide member 3220 as the seal-forming structure 3100 is brought into contact with the face. For example, the adhesive surface 3102 may be oriented to angle away, or it may curl away from the patient's face, for example under its own weight. One advantage of this arrangement is that the patient interface 3000 can be positioned by adjusting it until the right position is achieved, using the positioning guide member 3220 as a guide, and the adhesive surfaces 3102 do not contact any part of the face during this process. This configuration may also allow the patient 1000 to locate the position of the seal-forming structure 3100 on their face correctly using only one hand, for example by bracing the positioning guide member 3220 against the face with one hand and then pressing the seal-forming structure 3100 against the facial skin firmly with the same hand.

In certain forms the plenum chamber 3200 may be generally formed in a shape that complements the shape of a patient's nose adjacent to which it will be positioned in use. In such forms, the portion(s) of the plenum chamber 3200 proximal to the patient 1000 when the patient interface 3000 is worn may be considered positioning guide member(s) 3220.

### 4.3.2.2 Plenum Chamber Materials

In certain forms of the present technology, the plenum chamber 3200 is constructed from a relatively hard material, e.g. polycarbonate.

In certain forms the plenum chamber 3200 is formed from a material and with a structure that makes the plenum chamber 3200 relatively rigid and able to maintain its form when pressurised with typical therapeutic pressures. The plenum chamber 3200 may provide strength and structure to the patient interface 3000.

In other forms the plenum chamber 3200 is formed from a softer and/or more flexible material, for example silicone or a thermoplastic elastomer (TPE). Some of the advantages of TPE described above in relation to its use for the seal-forming structure 3100 may also apply for a plenum chamber 3200 formed from TPE in certain forms of the technology. In addition, forming the plenum chamber 3200 from a flexible material, such as TPE, allows the walls of the plenum chamber 3200 to deform. A further advantage of a deformable plenum chamber is explained later in the specification.

### 4.3.2.3 Plenum Chamber Inlet Port

In certain forms of the technology, for example as shown in FIGS. 4A, 4B, 4C, 5A, 10A, and 10E, the plenum chamber 3200 comprises a plenum chamber inlet port 3202. Other forms of the technology illustrated in the figures also have a plenum chamber inlet port 3202 but it is not labelled in every figure. The flow of breathable gas may be configured to be conveyed to the plenum chamber 3200 through the plenum chamber inlet port 3202. In certain forms the plenum chamber inlet port 3202 is configured to be directly or indirectly attached to the air circuit 4170. In the exemplary forms of the technology, the plenum chamber inlet port 3202 is preferably located at the second end of the plenum chamber 3200. The plenum chamber inlet port 3202 may be formed as an opening in the body of the plenum chamber 3200.

### 4.3.3 Integral Seal-Forming Structure and Plenum Chamber

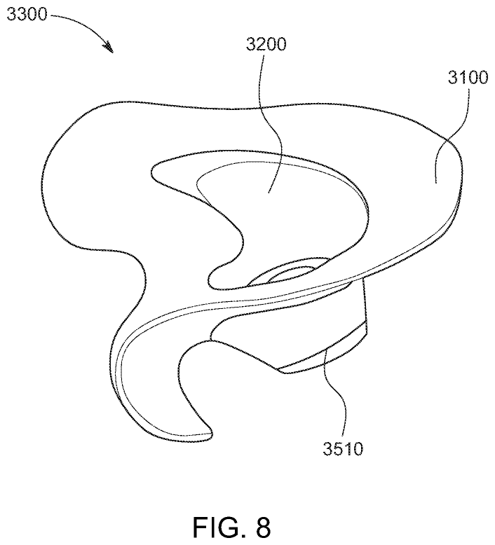

In certain forms of the technology, the plenum chamber 3200 and the seal-forming structure 3100 are integrally formed together as a single component 3300. An exemplary such form is shown in FIG. 8.

The advantages of integrally forming the plenum chamber 3200 and the seal-forming structure 3100 as a single component 3300 may include reduced part count, ease of manufacturing, and reduced packaging.

### 4.3.4 Connection Port

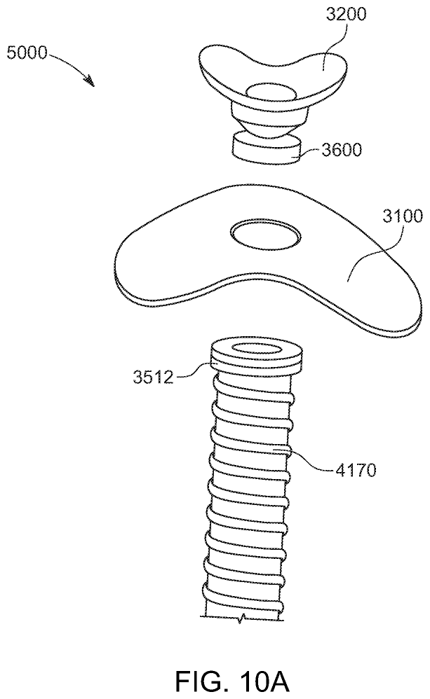

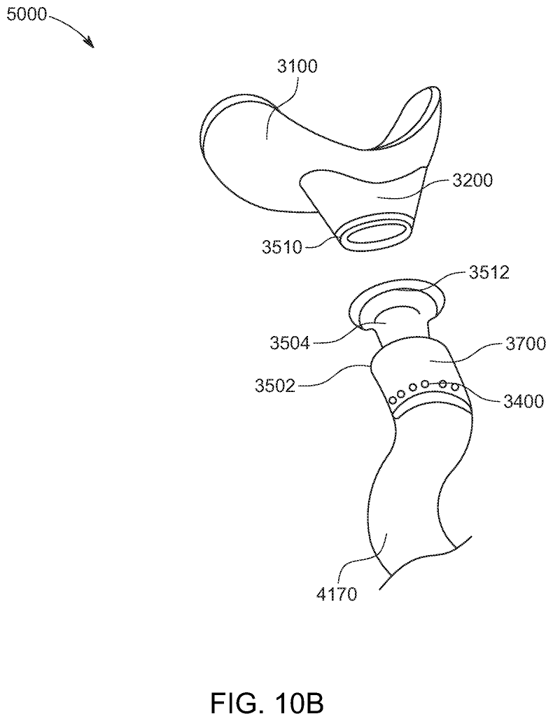

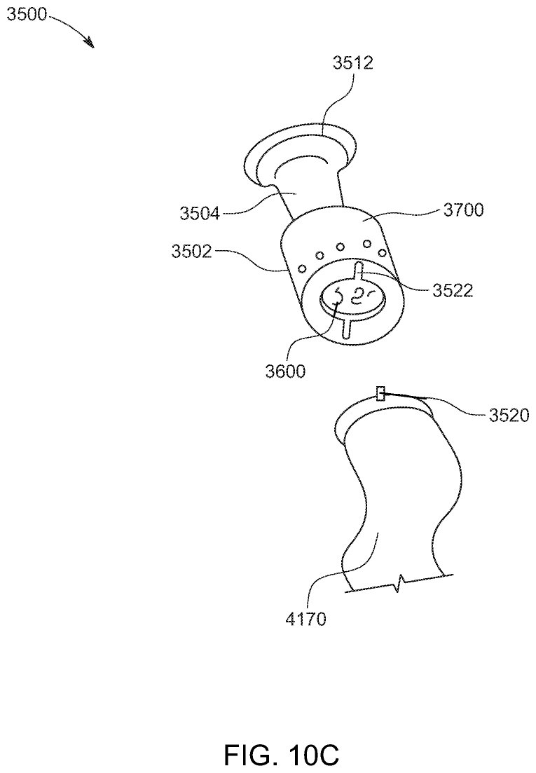

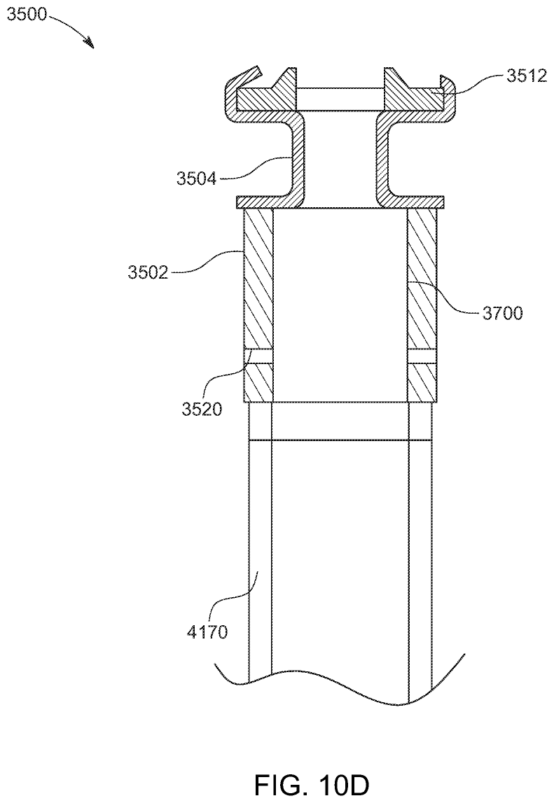

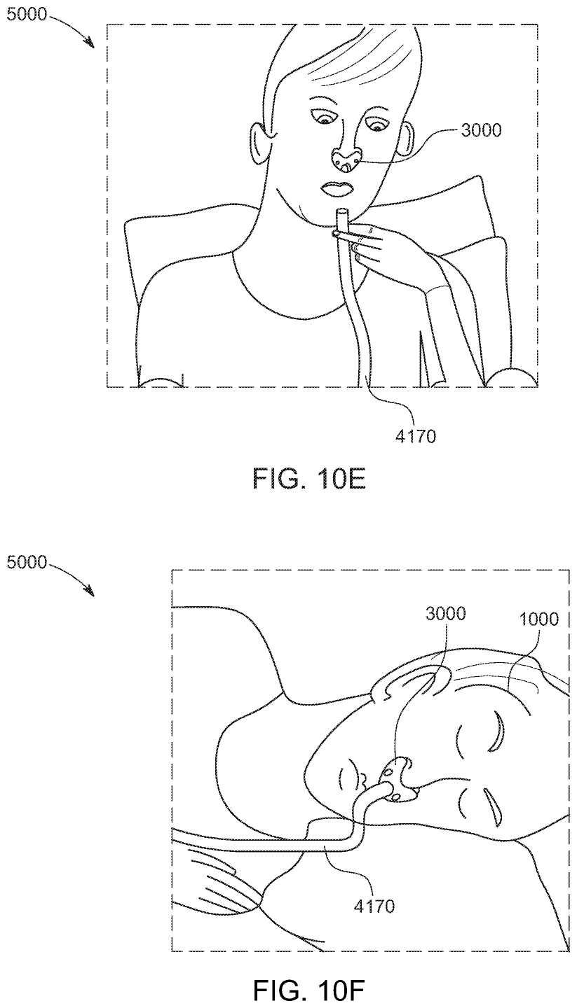

In certain forms of the technology, for example in the forms shown in FIGS. 10B, 10C and 10D, the patient interface 3000 further comprises a connection port 3600 which, in use, may be connected to the air circuit 4170 to convey the flow of breathable gas from the air circuit 4170 to the plenum chamber 3200 through the plenum chamber inlet port 3202. In other forms of the technology, the connection port 3600 is the same opening as the plenum chamber inlet port 3202. The connection port 3600 is not labelled in every figure.

In certain forms, such as shown in FIGS. 10B to 10D, the patient interface 3000 comprises a tube 3502 having a first end and a second end, where the first end of the tube is fluidly connected to the plenum chamber inlet port 3202 and the second end of the tube comprises the connection port 3600. The first and second ends of the tube 3502 are opposite to each other. The first end is configured to connect with the inlet port 3202 of the plenum chamber 3200. The second end forms the connection port 3600.

In one form, the first end of the air circuit 4170 may be connected to the second end of the tube 3502 (i.e., the connection port 3600) through a fastener. In the illustrated embodiment of FIG. 10C, for example, the fastener is a twist-lock, or a bayonet fastener where the air circuit 4170 has prongs 3520 which are inserted into a receiver 3522 of the tube 3502. Alternatively, the tube 3502 and the air circuit 4170 may be formed together.

### 4.3.5 Vent

In certain forms of the technology, the patient interface 3000 comprises a vent 3400 constructed and arranged to allow for the washout of exhaled gases, e.g. carbon dioxide. The vent 3400 may be implemented through a vent structure, which may be formed or provided in any one or more components of the patient interface 3000.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO₂ by the patient while maintaining the therapeutic pressure in the plenum chamber in use.

One form of vent 3400 in accordance with the present technology comprises a plurality of holes, for example, about 5 to about 80 holes, or about 10 to about 40 holes, or about 20 to about 25 holes.

In certain forms of the technology, for example as shown in FIG. 5A, the vent 3400 may be located in the plenum chamber 3200.

Alternatively, the vent 3400 may be located in a tube 3502 fluidly connected to the plenum chamber 3200 as described above. In still other forms, the vent 3400 may be located in a decoupling structure (such as described in further detail below), e.g., a swivel. In other forms, for example the forms of FIGS. 10B and 10C, the vent may be provided in a heat and moisture exchanger 3700, as described in more detail below.

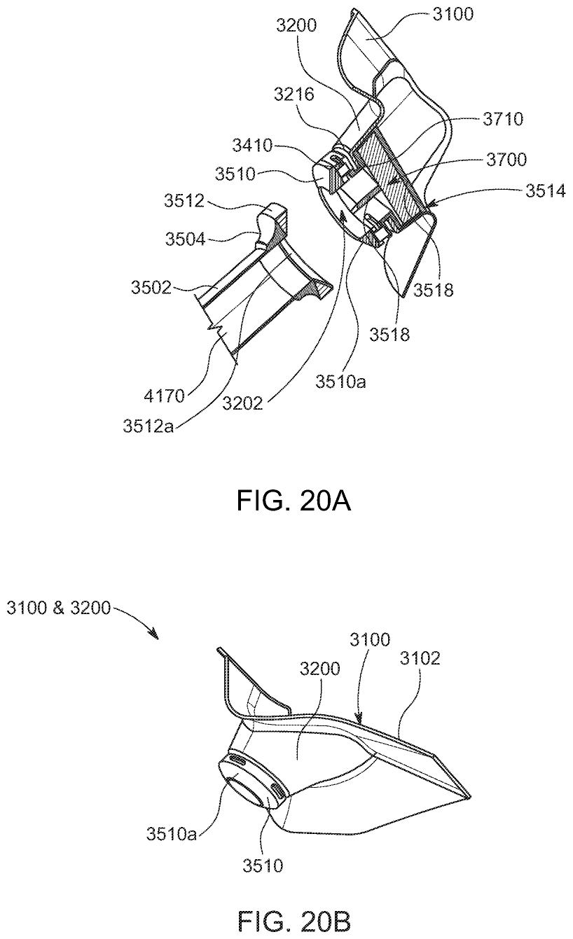

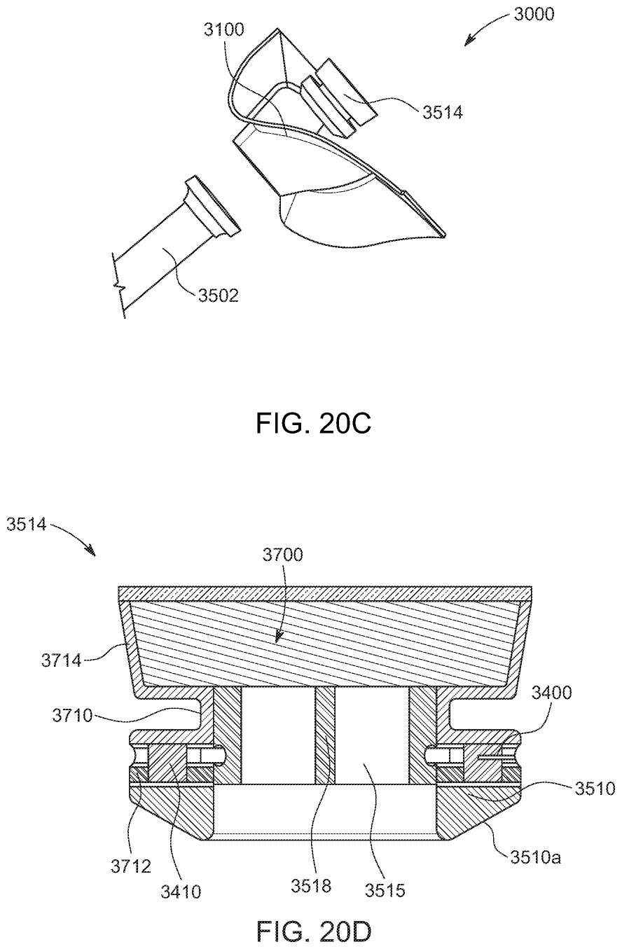

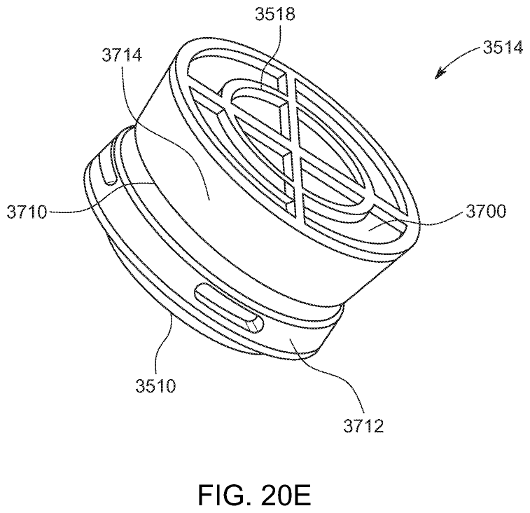

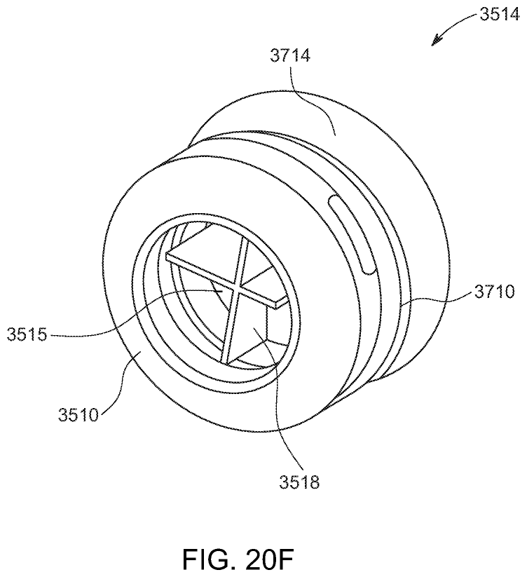

In certain forms, for example as illustrated in FIG. 20D, the patient interface 3000 further includes a diffuser 3410 which is configured to diffuse the vent flow of gases from the interior of the plenum chamber 3200 to ambient. The diffuser may be located in a channel in the vent 3400. For example, in the form illustrated in FIG. 20D, the diffuser 3410 comprises a body of diffuser material located in a chamber along a channel of vent 3400.

The advantages of the diffuser 3410 is that it reduces noise generated by vent flow, and prevents jetting. When jets of air are expelled through vents, these jets may be directed towards a sleeping partner or cause noise by contact with nearby surfaces. The noise and jetting may disturb the patient 1000 and/or the sleeping partner in their sleep, thereby reducing their quality of sleep, or cause other discomfort.

### 4.3.5.1 Ports

In certain forms of the present technology, a patient interface 3000 includes one or more ports that allow access to the volume within the plenum chamber 3200. In certain forms this allows a clinician to supply supplementary oxygen. In one form, this allows for the direct measurement of a property of gases within the plenum chamber 3200, such as the pressure.

### 4.3.5.2 Breath-to-Atmosphere Vent

In certain forms of the technology, the patient interface 3000 may include a vent 3400 configured to be able to adopt at least two configurations. In one configuration, which may be termed an open configuration, the vent 3400 allows the patient to inhale and exhale through the vent 3400 without significant impedance, or with a level of impedance that is largely unnoticeable by the patient. In another configuration, which may be termed a closed configuration, the vent 3400 is more occluded than in the open configuration. In some forms, in the closed configuration, the vent 3400 allows the washout of exhaled gases from an interior of the plenum chamber 3200 to ambient whilst substantially maintaining the pressure within the plenum chamber as positive with respect to ambient. In other forms, in the closed configuration, the vent may substantially block all washout of gases through the vent, and instead exhaled gases exhaust through a separate vent structure. Such a vent 3400 may be referred to as a “breathe-to-atmosphere” vent (BTA vent).

Whether the BTA vent adopts the open or closed configuration may be based on the pressure of the supply of breathable gas provided from the RPT device 4000 to the patient interface 3000. When no breathable gas is supplied, or when the flow of breathable gas is supplied at a pressure below a certain threshold, for example below a therapeutic pressure level such as 6 cmH₂O, the BTA vent may be configured to adopt the open configuration. When the flow of breathable gas is supplied at a pressure above a certain threshold, for example above a therapeutic pressure level such as 6 cmH₂O, the BTA vent may be configured to adopt the closed configuration.

A further explanation of a patient interface system comprising a vent that may be considered to act in the manner of a BTA vent, as described above, is provided in PCT Application No. PCT/US2012/055148, the contents of which are hereby incorporated by reference.

In one application, a BTA vent may be used in a patient interface system in which the BTA vent is configured to adopt the open configuration when the patient first dons the patient interface 3000 and while the patient is detected as being awake by the RPT device 4000. In this configuration, the RPT device may not supply a flow of breathable gas, or may be configured to provide a small flow of breathable gas to help flush out exhaled CO₂ from the plenum chamber 3200. Once the RPT device 4000 detects that the patient has gone to sleep, the flow of breathable gas may be supplied at a therapeutic pressure, which causes the BTA vent to adopt the closed configuration.

The patient interface 3000 having a seal-forming structure 3100 that adheres to the patient's face has several advantages over conventional patient interfaces 3000 with respect to BTA vents.

In exemplary forms of such patient interfaces 3000, the volume within the plenum chamber 3200 may be relatively small compared to some patient interfaces that are held on the face by other means, for example headgear straps. This volume, or a related volume, may be referred to as the “deadspace” within the patient interface. The small volume of the plenum chamber 3200 means that, when the patient breathes through the BTA vent in the open configuration, the breathing is unlikely to feel stuffy. Furthermore, if, when the BTA vent is in the open configuration and the patient is awake, a small flow of breathable gas is supplied by the RPT device 4000 to assist in CO₂ washout, the small volume means that the flow rate may be lower than would otherwise be required to achieve the same level of CO₂ washout if the volume in the plenum chamber 3200 was larger. These factors may help to minimise or reduce the flow rate when the patient is wearing the mask and still awake, reducing discomfort and helping the patient get to sleep.

One challenge with patient interfaces that are used with BTA vents and where the RPT device increases the pressure of the supply of breathable gas once the patient is detected as sleeping, is that the desired tension of headgear straps changes as the pressure inside the plenum chamber increases. The higher the pressure inside the plenum chamber, the greater the force acting to push the patient interface off the patient's face. The positioning and stabilising structure counteracts this force, and this is conventionally implemented through headgear straps having the necessary tension. In such a system, patients sometimes put the patient interface mask on when awake with headgear tension that is sufficient to keep the patient interface in sealed contact with the face when breathable gas is supplied at low pressure, or not supplied at all, but the tension may be insufficient to maintain the sealed contact of the patient interface with the face when the breathable gas is supplied at a higher, e.g. therapeutic, pressure. A patient interface 3000 such as described in forms of the technology herein avoids this issue because the seal-forming structure 3100 is adhered to the patient's face with an adhesive. Provided the adhesive is selected to be sufficiently strong to maintain the patient interface 3000 in place when gas is supplied at therapeutic pressures, the patient interface 3000 will remain in place with a good quality seal even as the pressure of the gas increases once the patient is asleep.

In addition, it has been observed that a seal-forming structure 3100 that adheres to a region around the patient's nares may be less likely to occlude a patient's nares than some conventional patient interfaces that supply a flow of gas to the nares and are maintained on the face using headgear strap tension, which can act to close the nares. This means that a patient may find it easier to breathe while wearing a patient interface 3000 with a seal-forming structure 3100 that adheres to the patient's face when there is no supply of breathable gas, or the supply is at a low pressure, as compared to some conventional patient interfaces.

### 4.3.5.2.1 Anti-Asphyxia Valve

One form of BTA vent is an anti-asphyxia valve (AAV) which is conventionally used in patient interfaces which cover both the nose and mouth as a measure to mitigate the risk of asphyxiation. The AAV ensures ventilation to the airways of the patient 1000 in case of disruption of the supply of breathable gas to the plenum chamber 3200 and/or the airways of the patient 1000. In certain forms of the present technology, a patient interface 3000 may comprise a conventional design of AAV acting in use as a BTA vent, as described above.

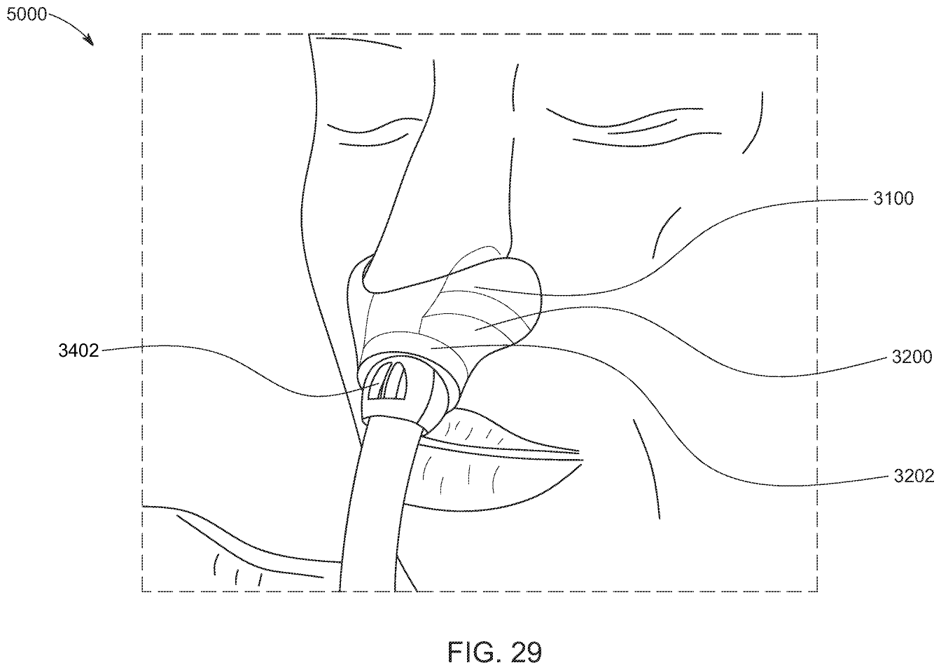

For example, in the form of the technology illustrated in FIG. 29, the patient interface 3000 comprises a vent 3400 that, in use acts as a BTA vent in the manner described above. The BTA vent may be in the form of an anti-asphyxia valve 3402. In the illustrated form of the technology, the anti-asphyxia valve 3402 is located on or proximate an end of the air circuit 4170 that connects to the inlet port 3202 of the plenum chamber 3200. In other forms, the AAV may be located in another location, for example in the plenum chamber 3200, or in a tube connected between the air circuit 4170 and the plenum chamber 3200.

The anti-asphyxia valve 3402 may comprise a flap which, when the valve 3402 is in the closed configuration, is configured to cover an opening in a wall of the air circuit 4170 and to prevent or limit leakage of gas contained in the plenum chamber 3200 in use. When the valve is in the open configuration, for example in case of a disruption or reduction in the flow of the breathable gas to the plenum chamber 3200, or if the pressure of the breathable gas supplied by the RPT device 4000 has not yet ramped up (e.g. if the patient is detected as still being awake), the flap is in a position where the opening is less occluded to allow the patient 1000 to breathe directly to and from the ambient atmosphere.

### 4.3.5.3 Expiratory Resistance Valve

In certain forms of the technology, positive pressure within the plenum chamber 3200 may be created in a manner other than through the supply of air into the plenum chamber 3200 from a RPT device 4000. For example, in one form of respiratory therapy system 2000, positive pressure is created in the plenum chamber 3200 from the flow of gases exhaled by the patient. Such a system may be referred to as an expiratory positive airway pressure (EPAP) system. EPAP systems according to certain forms of the technology may not comprise a RPT device or an air circuit as described herein. Instead, the EPAP system may comprise a vent that is configured to create and maintain the therapeutic pressure in the plenum chamber from the flow of gases exhaled by the patient.

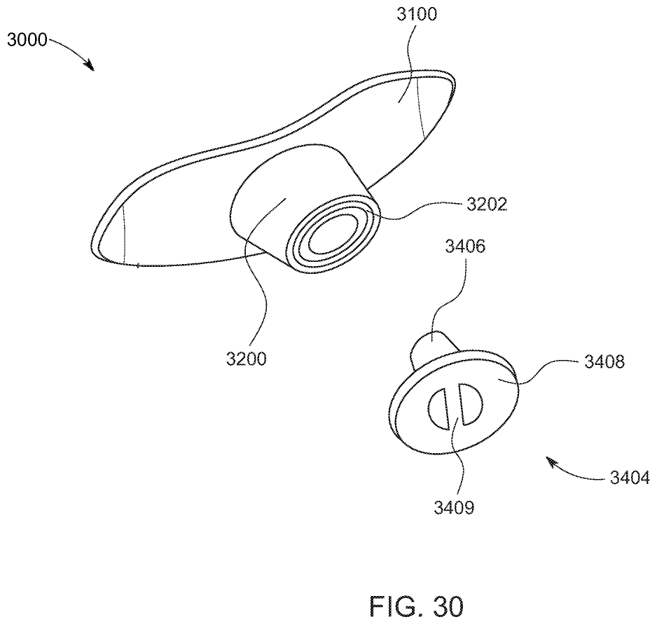

One exemplary form of a patient interface 3000 for use in an EPAP system is shown in FIG. 30. The patient interface 3000 of FIG. 30 comprises a plenum chamber 3200 and seal-forming structure 3100 of any of the forms of technology described elsewhere in this specification.

In the illustrated form, the patient interface 3000 further comprises a vent 3400 in the form of an expiratory resistance valve 3404. The expiratory resistance valve 3404 is configured to allow gas to flow in two directions through the valve. Gas may flow through the expiratory resistance valve 3404 in one direction, from the ambient atmosphere into the plenum chamber 3200, for breathing by the patient. The expiratory resistance valve 3404 is configured so that the flow of gas in this direction is sufficient for a patient to breathe. Gas may also flow through the expiratory resistance valve 3404 in the opposite direction, from the internal volume of the plenum chamber 3200 to ambient, to allow washout of exhaled gases from the patient. The expiratory resistance valve 3404 is configured so that the flow rate of gas permitted in this direction creates and maintains a positive pressure in the plenum chamber 3200 compared to the ambient pressure that is sufficient to provide a therapeutic effect on the patient.

In certain forms of the technology, the expiratory resistance valve 3404 is attached to, for example permanently attached to, the plenum chamber 3200.

In other forms, the expiratory resistance valve 3404 is provided to the plenum chamber 3200 in a removable manner. This may permit replacement and/or cleaning of the expiratory resistance valve 3404. For example, in the form shown in FIG. 30, the expiratory resistance valve 3404 is configured to be provided to the inlet port 3202 of the plenum chamber 3200. This configuration may allow the same assembly of plenum chamber 3200 and seal-forming structure 3100 to be interchangeably used with the expiratory resistance valve 3404, and to connect to an air circuit 4170 and to receive a flow of gas from a RPT device 4000 for the administration of respiratory therapy (e.g. CPAP), depending on the patient's preference.

The expiratory resistance valve 3404 may comprise a tube 3406 and a cap 3408. The tube 3406 may be configured to be inserted into the inlet port 3202 of the plenum chamber 3200 and the cap 3408 may be configured to substantially span across the inlet port 3202 when the tube 3406 of the expiratory resistance valve 3404 is inserted into the inlet port 3202 to prevent over-insertion. The cap 3408 may include valve components 3409, as shown in FIG. 30, which operate to permit the flow of gas into and out of the plenum chamber 3200 in use, and to control the pressure in the plenum chamber 3200 when the patient interface 3000 is in use, as described above.

While the patient interface 3000 of FIG. 30 may be used as a stand-alone apparatus for EPAP therapy, in alternative forms of the technology, the respiratory pressure therapy system 2000 may comprise a patient interface 3000 that receives a flow of breathable gas at a positive pressure from a RPT device 4000 and also an expiratory resistance valve 3404 to increase and help maintain the therapeutic pressure in the plenum chamber 3200. This may offer some benefits of both CPAP and EPAP systems, while reducing the level of power and/or size required for the RPT device 4000. For example, the air circuit 4170 may be attached to the inlet port 3202 with the expiratory resistance module 3404 being connected between the inlet port 3202 and the air circuit 4170.

### 4.3.5.4 Split-Vent

In some forms of the technology in addition to the vent 3400 constructed and arranged to allow for the washout of exhaled gases, which may be referred to as a first vent 3422, there may be a second vent 3424 configured to vent air received from the RPT device 4000 before the air is supplied to the patient interface 3000. The second vent 3424 may be configured and arranged to reduce the flow rate of air from the RPT device 4000 that is received by the patient interface 3000 whilst maintaining the pressure of the flow of air to the patient 1000. The second vent 3424 may be at a different location in the respiratory therapy system from first vent 3422.

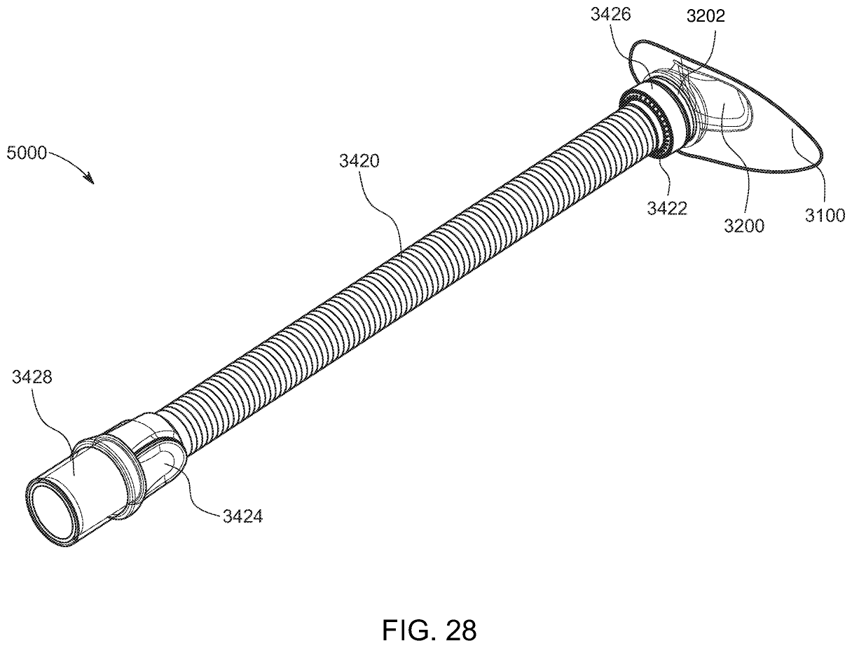

For example, in the form of the technology illustrated in FIG. 28, the patient interface system 5000 comprises a patient interface 3000 and a tube 3420. In use, breathable gas is supplied into the patient interface 3000 from the air circuit 4170 through the tube 3420. The tube 3420 comprises a first end 3426 and a second end 3428. The first end 3426 is connected to the inlet port 3202 of the plenum chamber 3200. The second end 3428 is configured to connect to the air circuit 4170. In some forms, the tube 3420 may be approximately 200-400 mm long, for example approximately 300 mm.

The first vent 3422 may be located at or proximate the first end 3426 of the tube 3420, for example on a ring-shaped cuff at the first end 3426 or on the patient interface 3000, for example on the plenum chamber 3200. The second vent 3424 may be located at or proximate the second end 3428 of the tube 3420. For example, the tube 3420 may comprise a cuff at its second end 3428 that is configured to connect to the air circuit 3170 and the second vent 3424 may be comprised as part of, or may connect to, the cuff.

The second vent 3428 may take the form of any of the vents described elsewhere in this specification, for example.

One advantage provided by the ‘split-vent’ configuration, such as in the form shown in FIG. 28, is that the diameter of tube 3420 that is required to deliver the same pressure or flow to the patient interface 3000 may be reduced compared to systems in which there is no second vent 3424. A narrower tube 3420 located proximate to the patient in use may be desirable for comfort and ease of use.

### 4.3.6 Nasal Prongs/Pillows

In one form of the technology shown in FIGS. 9A to 9D the patient interface 3000 comprises a pair of nasal puffs, nasal prongs or nasal pillows 3250. Each nasal pillow 3250 is constructed and arranged to form a seal with a respective naris of the nose of a patient 1000.

Nasal pillows 3250 in accordance with an aspect of the present technology include: a frusto-cone 3252, at least a portion of which forms a seal on an underside of the patient's nose, a stalk 3254, a flexible region on the underside of the frusto-cone 3252 and connecting the frusto-cone 3252 to the stalk 3254.

In one form, the nasal pillows 3250 of the present technology are formed by a nasal pillow body positioned inside the plenum chamber 3200, as shown in FIGS. 9A and 9B. The frusto-cone 3252 of the nasal pillow 3250 are formed at one end of the nasal pillow body and is configured to align with the first end of the plenum chamber 3200. The end of the stalk 3254 is formed at an opposite end of the nasal pillow body and includes an opening which is configured to align with the inlet port 3202 of the plenum chamber 3200 on the second end. The nasal pillow body is positioned such that, in use, breathable gas flows from the opening of the stalk 3254, to the frusto-cone 3252 and from thereon, to the naris of the patient 1000.

In one form, the plenum chamber 3200 and the nasal pillows 3250 are separate components that may be assembled by the patient 1000 by positioning the nasal pillow body inside the plenum chamber 3200. The nasal pillow body may be sized and shaped to fit snugly inside the plenum chamber 3200 to avoid cavities other than the air flow path inside the nasal pillow body. The nasal pillows 3250 can be slid out of the plenum chamber 3200 easily and this facilitates ease of cleaning the plenum chamber 3200 and/or the nasal pillows 3250.

In certain forms, the nasal pillows 3250 may be flexible. This improves patient comfort, especially when the patient interface 3000 is worn over an extended period of time.

The form of patient interface 3000 shown in FIGS. 9A to 9D comprises a seal-forming structure 3100 comprising an adhesive surface 3102 in addition to the nasal pillows 3250. The seal-forming structure 3100 may be similar to that described in relation to other forms of the technology and may comprise an adhesive surface 3102 that surrounds the nasal pillows 3250. The seal formed by the seal-forming structure 3100 may be additional to the seal formed by the nasal pillows and the nares of the patient. This may enhance the quality of the seal and may reduce the adhesion strength of the adhesive needed, since the nasal pillows 3250 may serve as a back-up seal in the event of loss of adhesion. In alternative forms, the nasal pillows 3250 are configured to provide little or no seal with the nares. In such forms, the nasal pillows nevertheless act to assist in positioning the patient interface 3000 in the correct position on the patient's face. This advantage is also provided in forms where the nasal pillows 3250 form a seal with the nares.

### 4.3.7 Decoupling Structure(s)

In certain forms of the technology, the patient interface 3000 includes one or more decoupling structures 3500 which may be configured to at least partly decouple the seal-forming structure 3100 from the air circuit 4170.

The decoupling structure 3500 may be configured to decouple (or insulate), either fully or partially, the adhesive attachment between the seal-forming structure 3100 and the patient's face from transmission of forces which are incident on the air circuit 4170 or another part of the respiratory therapy system during use. Such forces may include inadvertent tugs or pulls on the air circuit 4170 by the patient, a bed partner, or via contact with an object in the patient's surroundings, for example. If these forces are transmitted to the seal-forming structure 3100 directly, they may lead to the seal-forming structure 3100 either being ripped off from the patient's face or the seal-forming structure pulling hard at the patient's facial skin. Both of these scenarios can cause a significant amount of discomfort, pain and/or skin trauma.

The decoupling structure(s) 3500 of forms of the technology is/are configured to reduce the amount of force transmitted to the seal-forming structure 3100. In some forms, the decoupling structure enable disconnection of the air circuit 4170 and the plenum chamber 3200 when a force greater than a predetermined magnitude is incident on the air circuit 4170. Easy disconnection of the air circuit 4170 and the plenum chamber 3200 is also useful if a patient 1000 wants to disconnect from the respiratory therapy system 2000 temporarily, for instance, to go to the bathroom.

In this context, decoupling may be understood as insulating or reducing the effect of a force which is transmitted on a first component on a second component which is attached in some way to the first component. For example, the components may be the seal-forming structure 3100 and the air circuit 4170.

In certain forms of the technology the decoupling structure 3500 may form part of the plenum chamber 3200 or the air circuit 4170. Alternatively, as in the example of FIG. 10B, the air circuit 4170 and the plenum chamber 3200 may be connected through a decoupling structure 3500 located in between them. Exemplary decoupling structures 3500 will now be described. It will be understood that, although forms of the technology described in relation to some exemplary forms may only include one form of decoupling structure, in other forms a patient interface 3000 may comprise a combination of one or more of the exemplary forms of decoupling structure 3500.

### 4.3.7.1 Deformable Component

In certain forms, the decoupling structure 3500 may include a deformable component which is configured to deform when a first end of the air circuit 4170 is caused to tilt relative to its neutral position relative to the plenum chamber 3200 (i.e. the position it adopts in the absence of forces acting on the air circuit 4170). The first end of the air circuit 4170, as has previously been described, may be configured to connect to the plenum chamber inlet port 3202 and/or the connection port 3600.

The deformable component eliminates or reduces the amount of force transmitted to the seal-forming structure 3100 when the first end of the air circuit 4170 tilts in such a manner. The deformable component is especially useful to insulate the seal-forming structure 3100 from forces acting on the air circuit 4170 in a direction that is perpendicular to the longitudinal axis of the air circuit 4170.

### 4.3.7.1.1 Deformable Plenum Chamber

In one form, the deformable component is the plenum chamber 3200. In such a form, the plenum chamber may be formed in a manner whereby the plenum chamber is substantially flexible, for example the plenum chamber 3200 may be formed from a soft material and/or have thin walls. For example, the plenum chamber 3200 may be formed from a relatively thin layer of silicone or TPE.

In such a form, the flexible plenum chamber 3200 may be configured to distort when a force pushes the first end of the air circuit 4170 in a direction perpendicular to its longitudinal axis (i.e. the first end of the air circuit 4170 tilts relative to its neutral position). For example, portions of the plenum chamber 3200 may be configured to crumple, fold and/or stretch when the position of the air circuit 4170 is disturbed.

In one form, the plenum chamber 3200 may be configured so that, when a force causes the air circuit 4170 to tilt, a side of plenum chamber 3200 which lies on one side of the air circuit 4170 may compress or fold, and a side of the plenum chamber 3200 which is lies on an opposite side of the air circuit 4170 to the first side may stretch.

### 4.3.7.1.2 Tube

In certain forms of the technology, for example the exemplary form shown in FIGS. 10B to 10D, the decoupling structure 3500 comprises a tube 3502 which further includes a flexible section 3504 which is formed to be substantially flexible. For example, the flexible section 3504 may be more flexible than other sections of the tube 3502 and/or the patient interface 3000.

The flexible section 3504 may be formed of a material that is softer than the material used to form other parts of the tube 3502. Additionally, or alternatively, the flexible section 3504 may be formed from a thinner material than used to form other parts of the tube 3502.

In the illustrated exemplary form, the flexible section 3504 is in the form of a deformable neck which has a cross-sectional diameter that is narrower than the cross-sectional diameter of the other sections of the tube 3502. This makes the flexible section 3504 more prone to flexing than other regions of the tube 3502.

The flexible section 3504 acts to flex when a force is imparted on the air circuit 4170 to cause its proximal end to move laterally and this flex occurs before a lateral force is transmitted to the seal-forming structure 3100, thereby acting to at least partially decouple the seal-forming structure 3100 form the air circuit 4170.

### 4.3.7.2 Ball Joint

In one form (not shown in the Figs.) the decoupling structure may include a swivel or a ball joint which is configured to permit the rotational movement of the air circuit 4170 relative to the plenum chamber 3200 in order to reduce the amount of force(s) in a direction perpendicular to the longitudinal axis of the air circuit 4170 from being transmitted from the air circuit 4170 to the seal-forming structure 3100.

In one form (not shown in the Figs.), the ball joint may be a quick-release ball joint which is configured to detach the air circuit 4170 from the plenum chamber 3200 on application of a force (with force components in any direction) greater than a predetermined value on the air circuit 4170. The ball joint may detach on application of a force greater than a predetermined in value in a direction perpendicular to the longitudinal axis of the air circuit 4170 and/or in a direction parallel to the longitudinal axis of the air circuit 4170.

### 4.3.7.3 Magnetic Coupling

In certain forms of the technology, the decoupling structure 3500 comprises a first magnetic member 3510 provided to the connection port 3600. The first magnetic member 3510 is configured to be magnetically coupled to a second magnetic member 3512 that is provided to the air circuit 4170 when the air circuit 4170 is connected to the connection port 3600. The first magnetic member 3510 may be configured to separate from the second magnetic member 3512 when the air circuit 4170 is pulled away from the patient interface 3000 with a force greater than a magnetic force of attraction between the first magnetic member 3510 and the second magnetic member 3512.

One exemplary form of such a decoupling structure 3500 is shown in FIGS. 10A,10E and 10F. The decoupling structure 3500 provides connection between the connection port 3600 and the first end of the air circuit 4170 through magnetic coupling between a first magnetic member 3510 (not shown) and a second magnetic member 3512. In this form of the technology, the plenum chamber inlet port 3202 forms the connection port 3600.

In a second exemplary form, as shown in FIGS. 10B, 10C and 10D, the decoupling structure 3500 comprises a magnetic coupling between the first end of the tube 3502 and the plenum chamber inlet port 3202, which are connected through a magnetic coupling between a first magnetic member 3510 provided to the plenum chamber inlet port 3202 and a second magnetic member 3512 provided to the first end of the tube 3502. In such a form, the plenum chamber inlet port 3202 may be considered to form the connection port 3600.

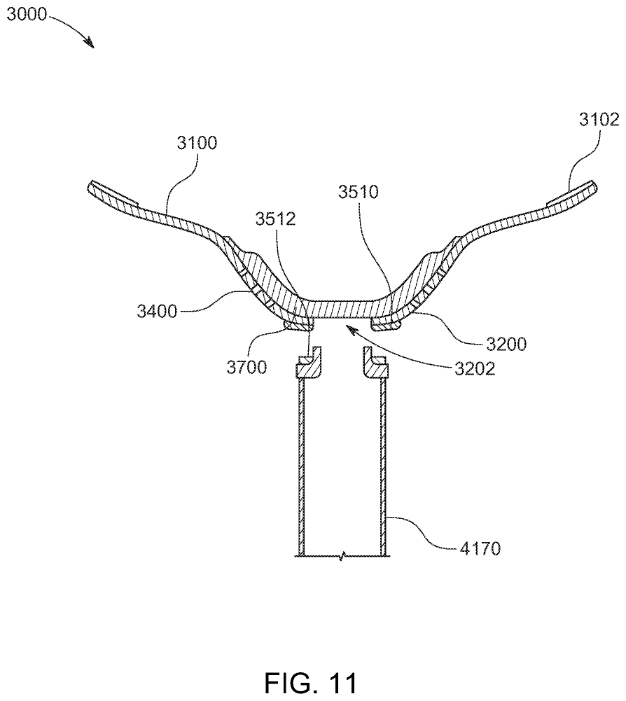

In further exemplary form, as shown in FIG. 11, the decoupling structure 3500 comprises a magnetic coupling between the end of the air circuit 4170 and the plenum chamber inlet port 3202, which are connected through a magnetic coupling between a first magnetic member 3510 provided to the plenum chamber inlet port 3202 and a second magnetic member 3512 provided to the end of the air circuit 4170.

The first magnetic member 3510 and the second magnetic member 3512 are configured to be magnetically coupled to each other in use. For example, the first and second magnetic members may both be magnets, e.g. ferromagnets, with the polarities of the first magnetic member 3510 and the second magnetic member 3512 being similarly aligned so that they attract each other. Alternatively, one of the first or second magnetic members may be a magnet and the other of the first or second magnetic members may be a non-magnet material which is attracted to the magnet, for example a metal able to be attracted to a magnet.

The magnetic members may be any suitable shape. In some forms, the first and second magnetic members 3510 and 3512 are ring-shaped and are positioned to encircle the opening in the respective component they form part of. This shape helps to ensure magnetic attraction around the perimeter of the connecting components and helps reduce gas leaks. In other forms, either or both the first and second magnetic members 3510 and 3512 may comprise a plurality of individual magnetic members arranged in an array, for example to surround the respective opening. In other forms, one or both of the first magnetic member 3510 and second magnetic member 3512 may comprise a plurality of individual magnets. The plurality of magnets may be arranged around the perimeter of the connection port 3600 and/or end of the air circuit 4170.

The magnetic coupling between the first magnetic member 3510 and the second magnetic member 3512 respectively allows the patient 1000 to easily connect and/or disconnect the air circuit 4170 from the patient interface 3000, for example by directly disconnecting the air circuit 4170 and/or tube 3502 if the tube 3502 is present. This ease of use is illustrated in FIGS. 10E and 10F.

In certain forms, the strength of the magnetic attraction between the first magnetic member 3510 and the second magnetic member 3512 respectively is selected so that they disconnect only on application of a force which is more than a pre-determined magnitude on the air circuit 4170. This force may be selected so that the air circuit 4170 is not able to disconnect too easily when subject to typical forces that are likely to be encountered during use, but disconnects if a force that is likely to cause significant discomfort to the patient is encountered. The pre-determined magnitude of force may be selected so that it is less than the amount of force required to pull the seal-forming structure 3100 away from the patient's face when it is adhered thereto.

In some exemplary forms of the technology, some parts of the patient interface 3000 are intended to be disposable and used only a limited number of times before being replaced. For example, in the exemplary form of the technology shown in FIG. 10B, the seal-forming structure 3100 and plenum chamber 3200 may form a single disposable component. There may be excessive manufacturing costs if a part that is intended to be disposable comprises a magnetic member, for example a metal component.

FIGS. 20A to 20F illustrate a patient interface 3000 according to an exemplary form of the technology that addresses this problem. In this form, the patient interface comprises a plenum chamber 3200 and seal-forming structure 3100 that are formed from a material that is cheap to manufacture and may also be recyclable, for example a thermoplastic elastomer (TPE). An adhesive is applied to the adhesive surface 3102 of the seal-forming structure 3100, for example by spraying.

The air circuit 4170 is connected to a tube 3502 and at the end of the tube 3502 is a magnetic member 3512. The tube 3502 may also comprise a flexible section 3502 as has been described previously.

In this form of the technology, magnetic coupling between the air circuit 4170 and the patient interface 3000 is achieved through an adapter 3514, which is shown in a perspective view in FIGS. 20E and 20F and in cross-sectional view in FIG. 20D. The adapter 3514 is configured to be removably inserted into, and to be retained within, the plenum chamber inlet port 3202 and to magnetically couple to the air circuit 4170 in order to retain the patient interface 3000 in connection with the air circuit 4170.

The adapter 3514 may be generally tubular in shape with a central bore 3515 to allow the passage of gas therethrough. The adapter 3514 may comprise a first flange 3712 and a second flange 3714 at each end. In between the first flange 3712 and the second flange 3714 is a neck 3710 that has a narrower diameter than the first and second flanges. The second flange 3714 may have a cross-sectional diameter that narrows continually from the end of the flange towards neck 3710 such that, as best seen in FIG. 20D, the second flange 3714 has a frustoconical shape.

The adapter 3514 is sized so that the adapter 3514 can be inserted into the plenum chamber inlet port 3202. When inserted, the flanges 3712, 3714 engage the plenum chamber inlet port 3202 in a retained, sealed arrangement, for example by a friction fit. In the illustrated form, the edges of the plenum chamber surrounding the plenum chamber inlet port 3202 are sandwiched between the flanges 3712 and 3714.

As described above, in certain forms, the plenum chamber 3200 may taper towards the plenum chamber inlet port 3202, for example to enable the portion of the plenum chamber 3200 proximate the patient to be wide enough to cover the patient's airways and the portion of the plenum chamber 3200 distal from the patient to be narrow enough to connect to the air circuit 4170. Accordingly, the plenum chamber 3200 may have sloping sides tapering away from the patient.

Correspondingly, the adapter 3514 may have sloped sides that are complementary to the sloped sides of the plenum chamber 3200 caused by this tapering. In the illustrated forms of FIGS. 20A to 20F, the second flange 3714 may have sloped sides tapering from a greater width distal from the air circuit 4170 (proximal the patient in use) and a narrower width proximal to the air circuit 4170 (distal the patient in use). In addition to providing a substantially effective fit with the plenum chamber 3200, the angle of the sloped second flange 3714 may enable the adapter 3514 to be slid easily into the plenum chamber inlet port 3202. The sloped second flange 3714 may also prevent the adapter 3514 from easily sliding out of the plenum chamber.

The plenum chamber inlet port 3202 may be able to distort to allow one of the flanges 3712, 3714 of the adapter 3514 to be inserted through it. In some forms, the plenum chamber inlet port 3202 may be provided with one or more small slits around its circumference in order to make such distortion easier. Where present, the slits may not be of sufficient size to create a leakage path for pressurised air inside the plenum chamber 3200.

In one form, as shown in FIG. 20E, the neck 3710 of the adapter 3514 takes the form of a groove extending around some or all of the perimeter of a mid-section of the adapter 3514 between the two ends. The neck 3710 is configured to receive the portion of the plenum chamber 3200 that forms the plenum chamber inlet port 3202 to secure the adapter 3514 to the seal-forming structure 3100 in use.

In certain exemplary forms, such as shown in FIG. 20E, the neck 3710 of the adapter 3514 forms an annular (i.e. circular) groove extending around all of the perimeter of a mid-section of the adapter 3514 between the two ends. The plenum chamber 3200 in this form may comprise an annular ring 3216 that forms the plenum chamber inlet port 3202.

The adapter 3514 may be pushed into the plenum chamber 3200 such that the annular ring 3216 of the plenum chamber 3200 interlocks with the neck 3710. The annular ring 3216 and the neck 3710 may be configured so that this interlock is substantially air-tight, i.e., the passage of gases from the air circuit 4170 through the adapter 3514 and the seal-forming structure 3100 does not leak through the region where the annular ring 3216 and the neck 3710 interlock.

In alternative forms, the neck 3710 of the adapter 3514 may be non-circular, for example oval, or another geometric shape, including shapes with rounded corners, and the plenum chamber inlet portion 3202 may be correspondingly shaped. Non-circular shapes may be useful for ensuring the adapter 3514 and the plenum chamber 3200 are connected with the components in a particular relative orientation. For example, this may help to orient the vent holes to avoid venting exhaled gases towards the patient. In alternative forms, the orientation between the adapter 3514 and the plenum chamber 3200 may be achieved through alternative structural features.

For example, in some forms, the adapter 3514 may include one or more protrusions and the plenum chamber 3200 may include one or more recesses are configured to be received by the protrusions (or vice versa). These arrangements may also prevent relative rotation between the adapter 3514 and the plenum chamber 3200 in use. By preventing relative rotation, the air circuit 4170 may be more difficult to twist, and thereby obstruct and/or damage, during use.

In an alternative form (not illustrated in any of the Figs.), the adapter 3514 may include a protrusion which is configured to be received by an indentation in the plenum chamber 3200 to secure the adapter 3514 to the seal-forming structure 3100 in use. The adapter 3514 may be mounted to the plenum chamber 3200 when the protrusion is received in the indentation such that the assembly of the adapter 3514 and the plenum chamber 3200 is airtight.

A magnetic member 3510 may be provided to one end of the adapter 3514. For example, the magnetic member 3510 may be provided to an end face of first flange 3712. This end of the adapter 3514 protrudes from the anterior side of the plenum chamber inlet port 3202 in use so that the magnetic member 3512 on the tube 3502 may connect to the magnetic member 3510 on the adapter 3514 in order to couple the air circuit 4170 to the patient interface 3000.

The magnetic members 3510 and 3512 on the adapter 3514 and tube 3502 respectively may be annular in shape and positioned to surround the respective openings that allow a flow of breathable gas from the air circuit 3170 into the plenum chamber 3200.

In certain forms, the connecting surfaces of the magnetic members may be oriented substantially orthogonally to the longitudinal axes of the adapter 3514 and tube 3502 respectively.

In other forms, for example as shown in in FIGS. 20A-20F, the connecting surfaces of the magnetic members may be at a non-orthogonal angle to the longitudinal axes of the adapter 3514 and tube 3502 respectively. For example, the magnetic member 3510 in FIGS. 20A-20F has a bevelled surface 3510a and the magnetic member 3512 has a correspondingly shaped surface 3512a. This shaping of the magnetically connecting surfaces helps facilitate magnetic connection between the adapter 3514 and the tube 3502 with the intended alignment.

In certain forms, one of the bevelled surfaces 3510a and 3512a of the magnetic members 3510 and 3512 may be convex while the other of the bevelled surfaces may be correspondingly concave in order to mate with the convex surface of the other magnetic member. In one form, the convex surface may be the bevelled surface 3510a of the adapter 3514 and the concave surface may be the bevelled surface 3512a of the tube 3502. In another form, the concave surface may be the bevelled surface 3510a of the adapter 3514 and the convex surface may be the bevelled surface 3512a of the tube 3502.

In other forms, the surfaces of the magnetic members 3510 and 3512 may be contoured with other shapes.

When the component comprising the plenum chamber 3200 and seal-forming structure 3100 is ready to be disposed of (for example because it is dirty) it can be thrown away or recycled while the adapter 3514 may be removed, retained and used with the replacement plenum chamber/seal-forming structure component. Nevertheless, the adapter 3514 may still need replacement if it becomes dirty and is difficult to clean.

In one form, the magnetic member 3510 may be made of a non-magnetised ferromagnetic material and the magnetic member 3512 may be a permanent magnet. This may be advantageous if the adapter 3514 needs to be replaced. In an alternative form, the magnetic member 3510 may be a permanent magnet, and the magnetic member 3512 may be a non-magnetised piece of ferrous metal. In another alternative form, both the magnetic member 3510 and the magnetic member 3512 may be permanent magnets.

In one exemplary form, the vent 3400 may be located on the adapter 3514. For example, the adapter 3514 may be provided with vent holes through its walls to fluidly connect the central bore 3515 with ambient in use to allow exhaled gases to be exhausted.

In the form illustrated in FIGS. 20A-20F, the vent holes are located in the first flange 3712. This is the flange that protrudes from the anterior side of the plenum chamber inlet port 3202 in use. In this form, the vent holes are arranged in a radial direction relative to the cylindrical geometry of the adapter 3514 in the illustrated form. A diffuser 3410 may be located in each of the vent holes to diffuse the vent flow of gas.

In one form, the adapter 3514 may include a frame 3518 which is best seen in FIGS. 20D and 20E. The frame 3518 may form the body of adapter 3514 and provide it with structure and rigidity to the adapter 3514. The frame 3518 may also form a recess or cavity configured to house a heat and moisture exchanger 3700, which will be described in more detail later.

In one form, the frame 3518 may be an integrally formed component. Alternatively, the frame 3518 may comprise a plurality of frame parts that are fitted together to form the frame 3518.

The adapter 3514 may also comprise a heat and moisture exchanger 3700 as will be described in more detail below.

In an alternative form (not shown in any of the Figs.), the adapter 3514 may be configured so that it, or a substantial part of it, is located outside the plenum chamber 3200. For instance, the adapter may be similar to the adapter 3514 shown in FIGS. 20D and 20E, but configured so that the end of the adapter that is opposite the end that couples to tube 3502 connects to the anterior surface of the plenum chamber, for example it may connect to a ring around the plenum chamber inlet port. In such a form, the adapter 3514 may not be formed with two flanges and a neck between them. One advantage of locating the adapter 3514 outside the plenum chamber 3200 is that no part of the adapter 3514 extends into the space inside plenum chamber 3200 where it risks coming into contact with the patient's nose. A component contacting the nose, particularly a hard component as adapter 3514 may be, may be uncomfortable and undesirable. Alternatively, the plenum chamber 3200 may need to be made larger to ensure such contact between the adapter 3514 and nose does not occur, which may also be undesirable. Furthermore, locating part of adapter 3514 inside the plenum chamber 3200 may stiffen the planum chamber 3200 and prevent it from deforming. As has been described above, a deformable plenum chamber 3200 may be desirable in some forms.

### 4.3.7.4 Hook-and-Loop Fastening

In certain forms of the technology (not shown in the Figs.), connection between the plenum chamber inlet port 3202 and the first end of the air circuit 4170 and/or the first end of the tube 3502 may be provided through hook- and loop-fasteners. In an alternative form of the technology, the seal-forming structure 3100 may be attached to a component forming the plenum chamber 3200 through a hook-and-loop fastening. For example, a non-patient contacting side of the seal-forming structure 3100 may be provided with hook or loop material and the perimeter of the plenum chamber 3200 may be provided with the complementary material (i.e. either loop or hook) so that the components attach together in use.

As with the magnetic coupling describe above, the hook-and-loop fastenings such as those describe above may be configured to detach when a force exceeding a pre-determined magnitude is exerted on the air circuit 4170, and the nature of the hook-and-loop fastening material may be selected accordingly (for example the density of hooks).

### 4.3.7.5 Tether

In certain forms of the technology, the patient interface 3000 may comprise, or be configured to be used with, a tether for tethering the air circuit 4170 relative to the patient 1000. For example, the tether may comprise a strap that is secured around a part of the patient's body, for example their neck or arm, and the tether may further comprise a clip on the strap that is configured to connect to a section of the air circuit 4170. The tether may be positioned in use so that the air circuit 4170 is not pulled tight when the tether is pulled away from the patient to its maximum extent. This means that any excessive force exerted on the air circuit 4170 to pull it away from the patient will be restricted by the tension in the tether when pulled tight against the patient's body.

### 4.3.8 Heat and Moisture Exchanger

In certain forms of the technology, the patient interface 3000 may comprise a heat and moisture exchanger 3700 which is configured to capture humidity and/or heat from gases exhaled by the patient 1000 and deliver humidity to the flow of breathable gas for breathing by the patient 1000.

The heat and moisture exchanger 3700 may comprise a body of heat and moisture exchanging material which absorbs heat and/or moisture from exhaled gases and releases the absorbed heat and moisture to incoming fresh breathable gas from the air circuit 4170.

In one form, a heat and moisture exchanger 3700 may include heat and moisture exchanging material which is deposited at a point along the flow of breathable gas. For instance, in the forms of the technology shown in FIGS. 10B, 10C, 10D the heat and moisture exchanging material is positioned inside the tube 3502. The heat and moisture exchanging material may occupy the full cross-section of a section of the tube 3502 so that gas cannot flow through the tube 3502 without passing through the heat and moisture exchanging material. The heat and moisture exchanging material 3700 may be deposited in a part of the volume inside the tube 3502 uniformly.

In another form, as shown in FIG. 11, the heat and moisture exchanging material 3700 is located on an inner wall of the plenum chamber 3200. For example, the heat and moisture exchanging material 3700 may be located in a cavity formed on the inner wall of the plenum chamber 3200.

It will be appreciated that the heat and moisture exchanging material may be positioned between the patient's airways and vent 3400 so that gas is not exhausted from the respiratory system without passing through the heat and moisture exchanging material 3700. For example, in the case of the forms of the technology shown in FIGS. 10B, 10C and 10D, the heat and moisture exchanging material is positioned in a part of tube 3502 that is upstream compared to the holes in the tube that form vent 3400 when considering the direction of flow of exhaled gases. In the case of the form of the technology shown in FIG. 11, in which the vent 3400 is formed by holes in the plenum chamber 3200, the heat and moisture exchanging material is positioned against the inner walls of the plenum chamber 3200 so as to cover the holes of vent 3400.

In the exemplary form of the technology shown in FIGS. 20A to 20F, a heat and moisture exchanger 3700 is comprised as part of the adapter 3514. For example, heat and moisture exchanging material may be located inside the central bore 3515 of the adapter 3514. In the illustrated form, heat and moisture exchanging material is located in the second flange 3714 in the end of the adapter 3514 that protrudes inwardly into the plenum chamber 3200 in use.

In an alternative form (not shown in any of the Figs.), the adapter 3514 may be configured so that the heat and moisture exchanger 3700 is located outside the plenum chamber 3200, but is still positioned in the path of the flow of gases entering the plenum chamber 3200. For instance, the heat and moisture exchanger 3700 may be comprised as part of an adapter that is located outside the plenum chamber 3200 in the manner explained above.

In certain forms of the technology, the heat and moisture exchanging material may comprise foams (e.g. unsalted or salted foams), paper, or non-woven materials. Other materials may be used in other forms of the technology, including any materials known conventionally for use in heat and moisture exchangers in respiratory systems.

### 4.3.8.1 Separate HME Module

In certain forms (not shown in any of the Figs.), the heat and moisture exchanger 3700 may be distanced from some other components of the patient interface, such as the plenum chamber 3200 and seal-forming structure 3100. In such forms, the patient interface 3000 may comprise a heat and moisture exchange (HME) module comprising the heat and moisture exchanger 3700. The HME module may comprise a housing that is physically separate from the plenum chamber 3200.

In such forms, a conduit may fluidly connect the plenum chamber 3200 with the HME module. The conduit may be connected to the plenum chamber 3200 and/or the HME module using any suitable connection mechanism, for example a magnetic coupling mechanism, including those described earlier. The HME module may also be fluidly connected to the RPT device by the air circuit 4170 by a similar or different mechanism.

In certain forms, the HME module may also comprise the vent 3400.

In certain forms, the HME module may comprise an engagement mechanism for retaining the HME module to a structure. The advantage of this arrangement is that the weight of the heat and moisture exchanger 3700 may be removed from bearing on the adhesion strength of the adhesive surface 3102. In certain forms, the structure may be the patient's apparel, for example apparel worn on the upper body, such as a shirt. This allows use of a relatively short conduit connecting the HME module and the plenum chamber 3200, reducing the risk of tangling.

### 4.3.9 Sensor Module

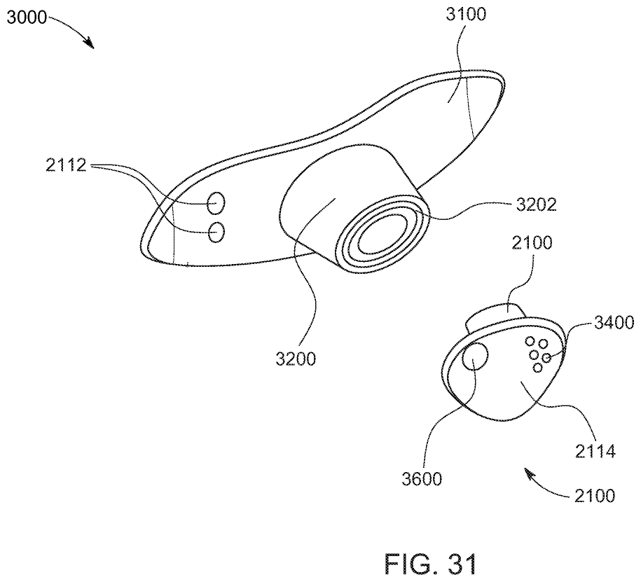

In certain forms, as illustrated in FIG. 31, the patient interface 3000 may be connected to a sensor module 2100 at the inlet port 3202. This allows the sensor module 2100 to detect characteristics of a patient's breathing and/or other characteristics of the patient's health and/or physiology. For instance, the sensor module 2100 may comprise a pulse oximeter to measure the oxygen level in the patient's blood. The sensor module 2100 may additionally, or alternatively, be configured to measure the flow rate of air inhaled or exhaled by the patient over time.

In certain forms, the sensor module 2100 may be configured to detect characteristics that may be used in determining a medical condition of the patient, for example to diagnose a respiratory condition, e.g. obstructive sleep apnea. The sensor module 2100 may comprise any sensor suitable to determine characteristics of the patient's breathing and/or condition necessary to make such a determination.

In the illustrated form, one or more electrodes 2112 may be provided on the seal-forming structure 3100 in a location where, when the patient interface 3000 is worn, the electrodes are in contact with the patient's skin. The electrodes 2112 may, in combination with a pulse oximeter, be configured to detect the oxygen levels of the patient's blood and relay the information to the sensor module 2100. For instance, the electrodes 2112 may be hydrogel contacts or other suitable pads.

In other forms, the sensor module 2100 is provided to the plenum chamber 3200 in a removable manner. This may permit replacement and/or cleaning of the sensor module 2100. For example, in the form shown in FIG. 31, the sensor module 2100 is configured to be provided to the inlet port 3202 of the plenum chamber 3200. This configuration may allow the same assembly of plenum chamber 3200 and seal-forming structure 3100 to be interchangeably used with the sensor module 2100, and to connect to an air circuit 4170 and to receive a flow of gas from a RPT device 4000 for the administration of respiratory therapy (e.g. CPAP).In one form, the sensor module 2100 may comprise a tube 2110 and a cap 2114. The tube 2110 may be configured to be inserted into the inlet port 3202 of the plenum chamber 3200 and the cap 2114 may be configured to substantially span across the inlet port 3202 when the tube 2110 of the sensor module 2100 is inserted into the inlet port 3202 to prevent over-insertion. The cap 2114 may include sensor components necessary to detect certain characteristics of the patient's breathing and/or condition in order to perform its function.

The sensor module 2100 may further comprise a vent 3400. For example, the vent 3400 may be provided in cap 2114. In the form of technology of FIG. 31, the vent 3400 comprises a vent structure configured to allow gas to flow in two directions through the vent. Gas may flow through the vent 3400 in one direction, from the ambient atmosphere into the plenum chamber 3200, for breathing by the patient. The vent 3400 is configured so that the flow of gas in this direction is sufficient for a patient to breathe. Gas may also flow through the vent 3400 in the opposite direction, from the internal volume of the plenum chamber 3200 to ambient, to allow washout of exhaled gases from the patient.

A patient interface 3000 according to forms of the technology comprising a sensor module such as is shown in FIG. 31 may not be used as part of a respiratory therapy system. Instead, it may be used as part of a respiratory diagnosis system. Such a respiratory diagnosis system may not comprise a RPT device or an air circuit as described herein.

In certain forms, the sensor module 2100 may comprise one or more processors configured to process the characteristics detected by its sensors. For example, the processors may be configured to analyse detected characteristics and/or diagnose a medical condition of the patient, for example OSA. In other forms, the sensor module 2100 may comprise a transmitter configured to transmit information detected by the sensors to a processor located remotely from the patient interface 3000. Any suitable wired or wireless communication protocol may be used, e.g. RF communication, Bluetooth, RFID, NFC, etc. In certain forms, the sensor module 2100 may comprise data storage device configured to store data indicative of characteristics detected by the sensors. The data storage device may store the data for later processing or transmission. Additionally, or alternatively, the data storage device may be able to be removed from the sensor module 2100 and taken to a processor where the data stored thereon may be transferred for processing. For example, the data storage device may be an SD card or the like.

### 4.4 Air Circuit

In one form, the patient interface system 5000 includes an air circuit 4170. The air circuit 4170 is configured to convey breathable gas to the patient interface 3000 for delivery to the airways of the patient 1000. For example, the air circuit 4170 of the form of the technology shown in FIG. 1 conveys the breathable gas from the RPT device 4000 to the plenum chamber 3200.

A first end of the air circuit 4170 may be connected to the plenum chamber inlet port 3202 and/or the connection port 3600. A second end of the air circuit 4170, which may be opposite to the first end, may be connected to an RPT device 4000.

In exemplary forms of the technology, the air circuit 4170 is flexible.

In certain forms, the air circuit 4170 may additionally include the conduit connecting an HME module to the plenum chamber 3200.

In certain forms, the geometric dimensions of the air circuit 4170 may depend on the flow parameters of the breathable gas supplied to the patient from the RPT device 4000. For instance, the diameter of the air circuit 4170 may be relatively small in the case of an RPT device 4000 configured to provide a supply of breathable gas at relatively low pressures (e.g. 2 to 6 cmH₂O), i.e. low pressure therapy. The diameter of the air circuit 4170 may be relatively larger for use with RPT devices 4000 configured to supply breathable gas at higher pressures (e.g. 6 to 20 cmH₂O).

### 4.4.1 Additional Tube

In certain forms, as illustrated in FIG. 27, the breathable gas is conveyed by the air circuit 4170 to a tube 3420 which itself conveys the breathable gas to the plenum chamber 3200. The diameter of the tube 3420 may be smaller than that of the air circuit 4170.

In one form, the patient interface 3000 may comprise the tube 3420. For example, the tube 3420 may be removably or permanently attached to the air inlet port 3202. In an alternative form, the air circuit 4170 may comprise the tube 3420, either integrally formed together or attached together (removably or permanently) to form a single assembly.

### 4.4.2 Positioning of Air Circuit

The patient interfaces 3000 described in relation to various forms of the technology herein may be accommodate the positioning of air circuits 4170 in various arrangements with respect to the patient.

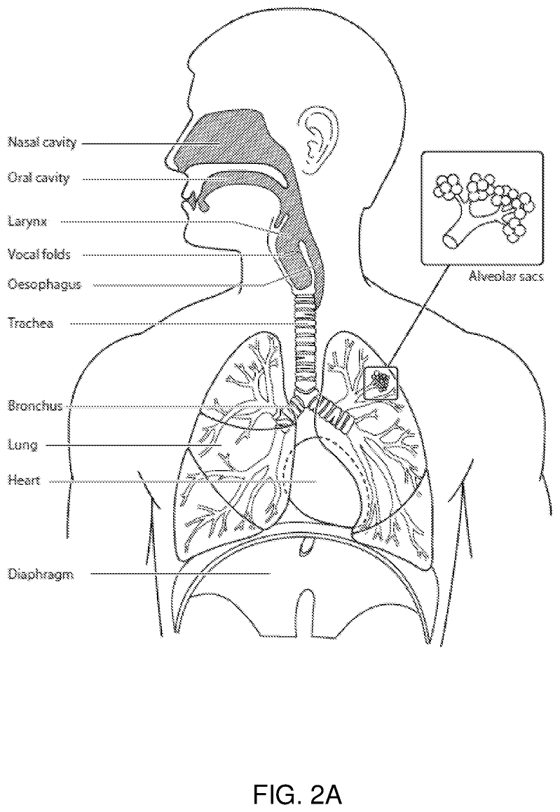

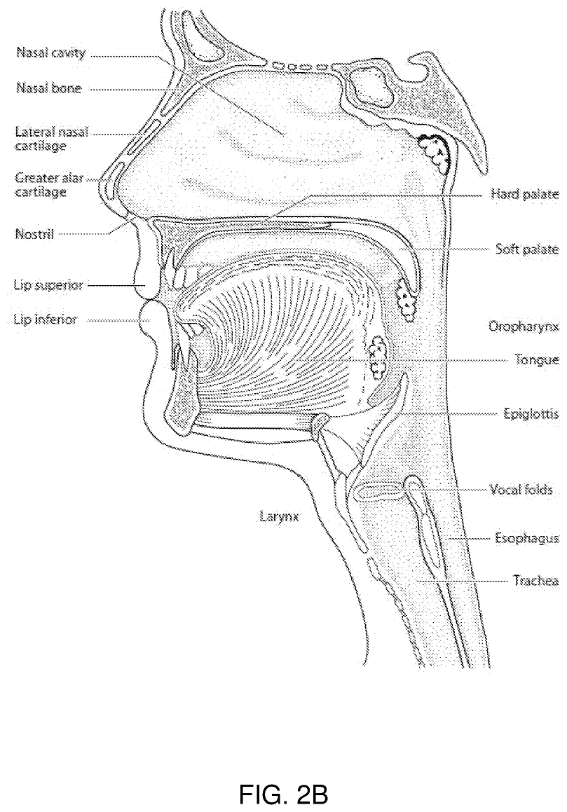

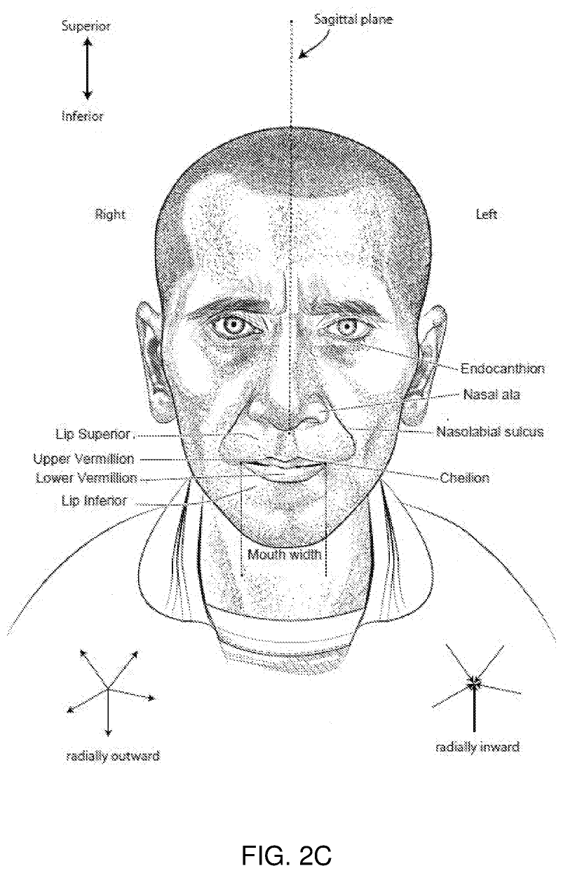

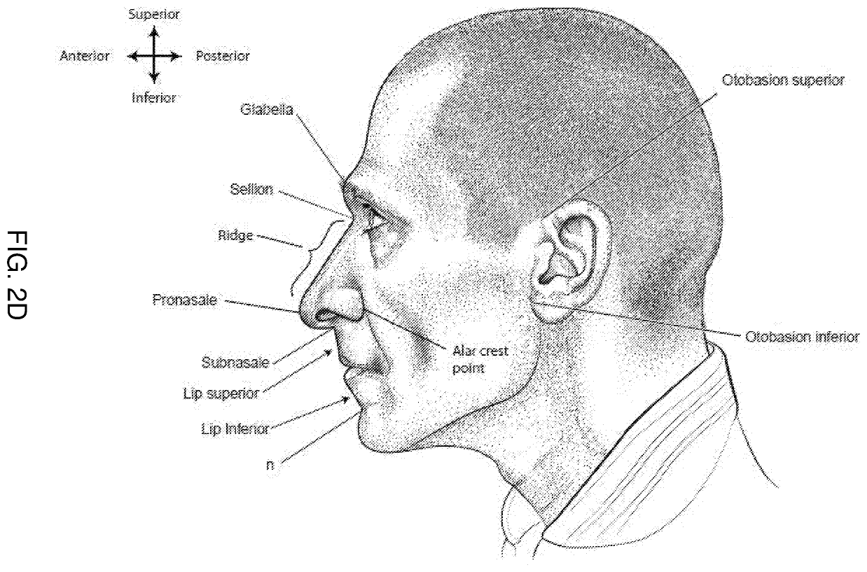

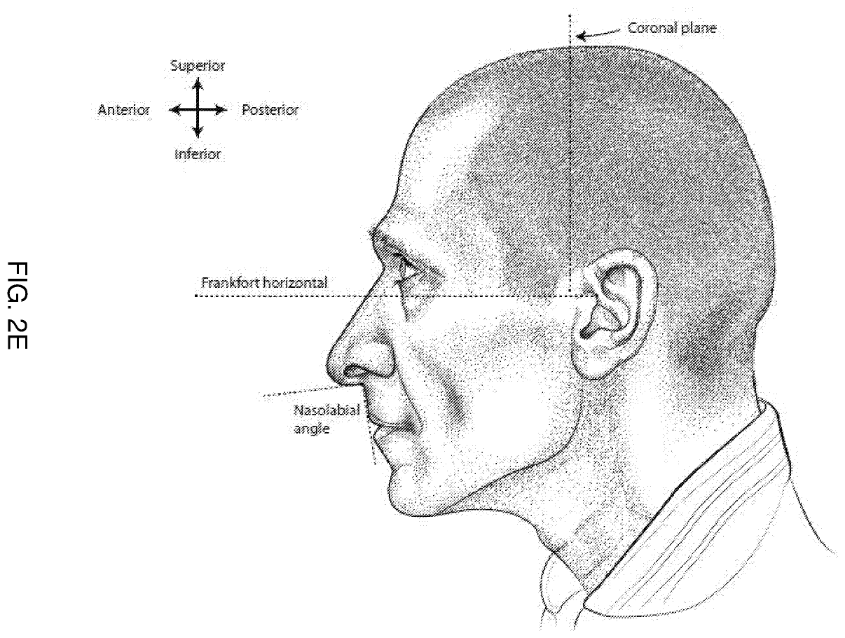

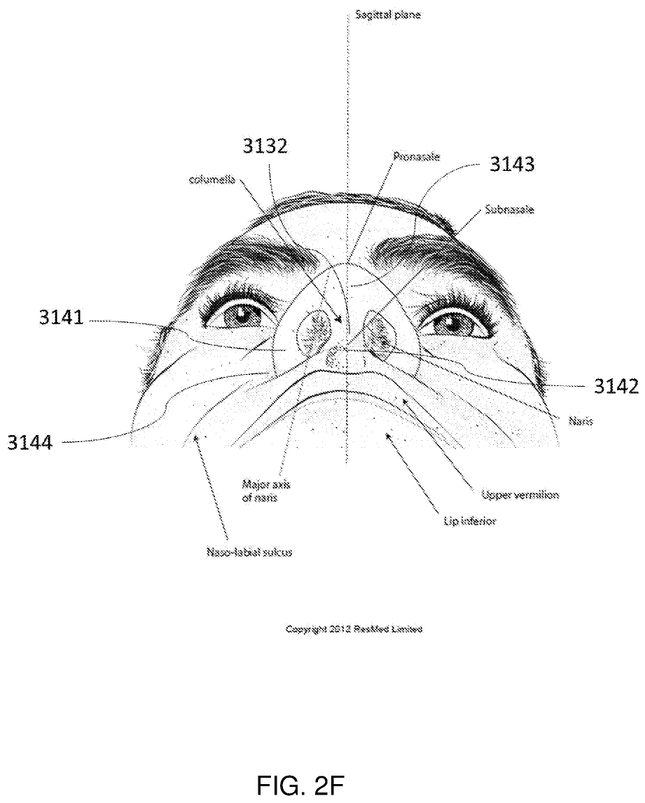

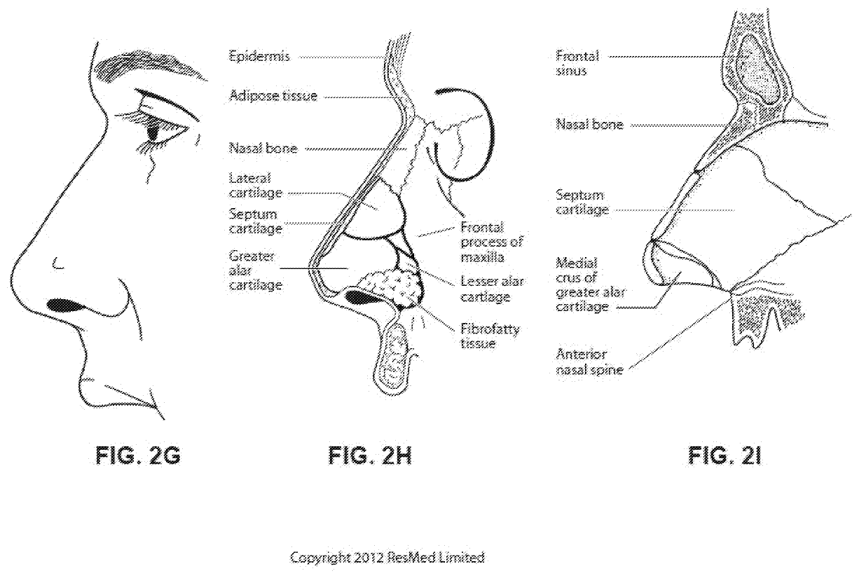

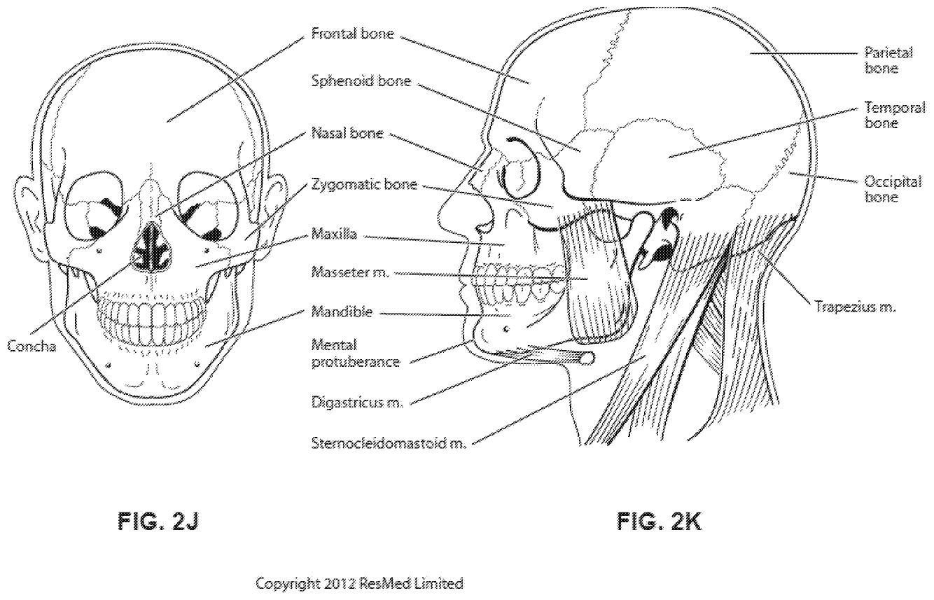

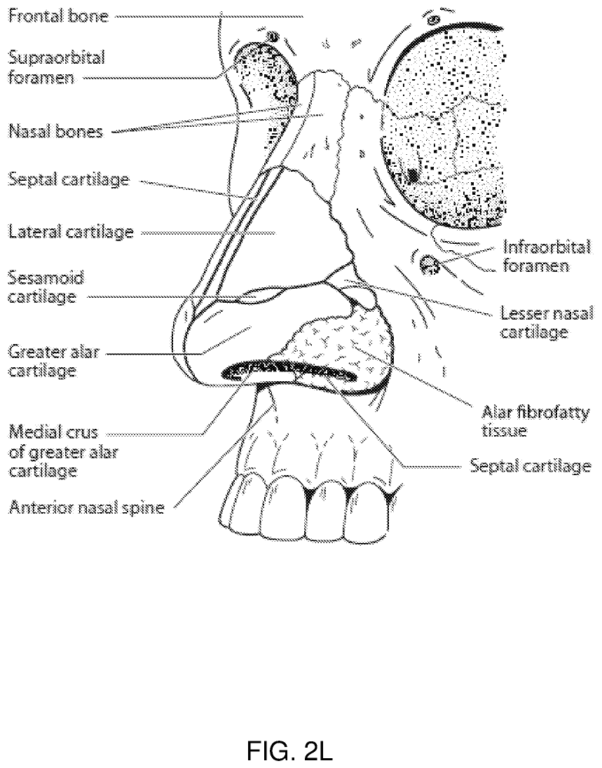

In certain forms, the air circuit 4170 may be configured to be routed to the patient interface 3000 from substantially above a transverse plane which is configured to pass through the patient's nasal and/or mouth regions, e.g. the Frankfort horizontal (see FIG. 2E). In such a form, the patient interface 3000 may comprise a positioning structure configured to hold part of the air circuit 4170 in relation to the patient's head in the desired position, for example the part of the air circuit 4170 may be held in a position superior to the patient's otobasion superior. For example, the positioning structure may comprise one or more straps configured to be worn on the patient's head and to engage the air circuit 4170. In one form, the air circuit 4170 may be configured to pass over the top and/or the back of a patient's head. Alternatively, or additionally, the air circuit 4170 may be configured to pass behind and/or close to the back of a patient's neck when the patient interface system 5000 is in use.

In another form, the air circuit 4170 may be configured to reach the patient interface 3000 from substantially below a transverse plane which is configured to pass through the patient's nasal and/or mouth regions, e.g. the Frankfort horizontal. Such an arrangement is shown in FIGS. 3A, 4A, 4B and 10F, for example. In this form, the patient interface 3000 may not need any positioning structure to secure the air circuit 4170 in place.

In one form, the air circuit 4170 may be routed around and/or proximal to one or both ears of the patient 1000. For instance, the air circuit 4170 may branch into two conduits before the end of the air circuit 4170 that connects to the patient interface 3000. Each of the conduits may be configured to be able to be passed behind respective ears of the patient 1000 in use.

### 4.5 Applicator

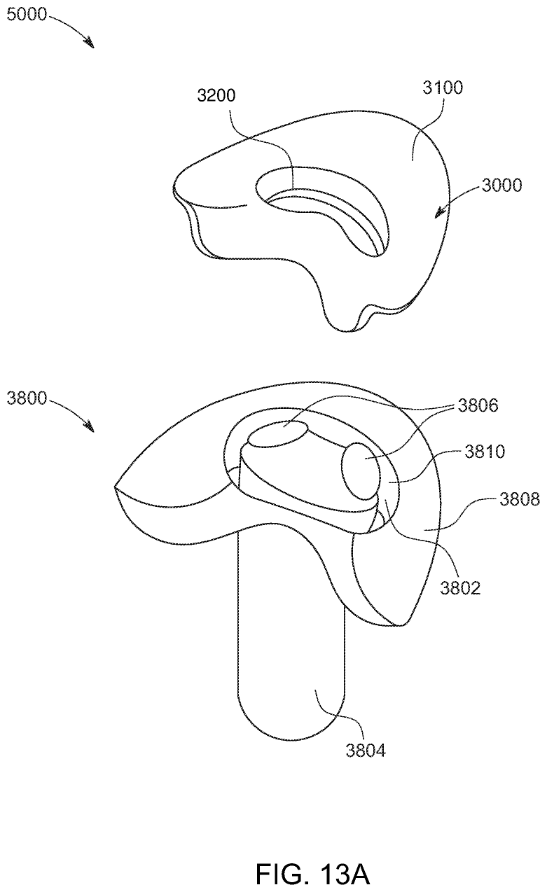

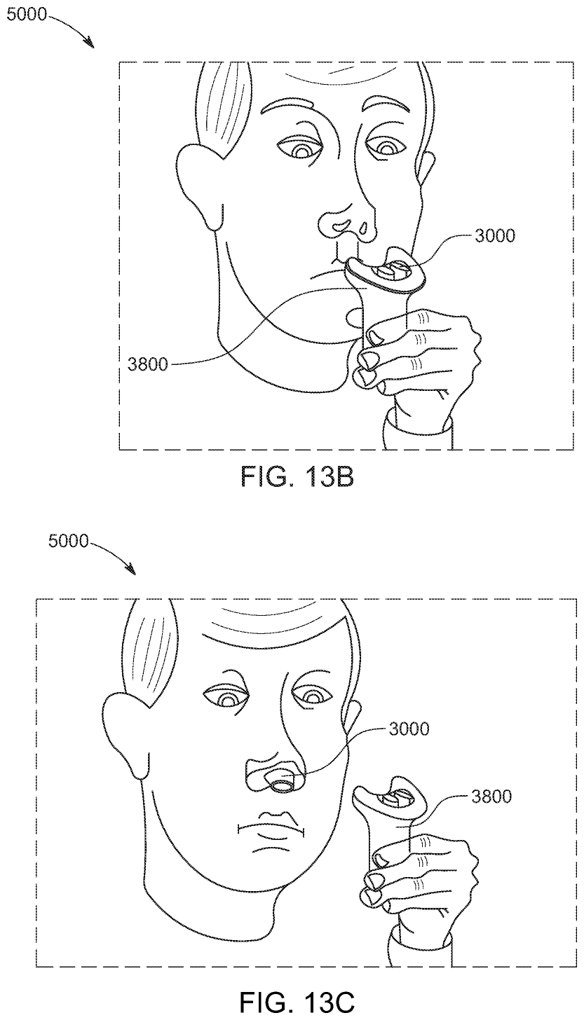

In certain forms of the technology, for example as shown in FIGS. 13A to 14C, 17 and 18A to 18C, the patient interface system 5000 includes an applicator 3800 which is configured to assist the patient 1000 in positioning the patient interface 3000 in a therapeutically effective position on the patient's face.

One of the advantages of the applicator 3800 is that it helps retain the seal-forming structure 3200 in shape from when the removable layer (or liner) 3104 is removed until the seal-forming structure 3100 is affixed to the patient's face. The applicator 3800 may be used as an alternative to or in addition to the shape retainer(s) 3140.

In certain forms, the applicator 3800 comprises a body 3810 comprising a recess 3802 configured to receive and retain the patient interface while the patient interface is put into the therapeutically effective position on the patient's face, a surface 3808 provided around the recess 3802 and a gripping portion 3804.

### 4.5.1 Body

In the exemplary forms of the technology, the applicator 3800 comprises a body 3810. Body 3810 may be formed from a material and having a structure so that the body 3810 is substantially rigid. For example, the body 3810 may be formed from a plastic material, for example polycarbonate. The body 3810 may be formed in a way that it may be molded, for example injection molded.

In exemplary forms of the technology, the body 3810 comprises a recess 3802 and a surface 3808, which will now be described.

In certain forms, the body 3810 may take the form of a front layer 3230, as described earlier in this specification, for example as shown in FIGS. 15B and 15C.

The body 3810, like the shape retainer(s) 3140 or the front layer 3230, is useful in maintaining the shape of the seal-forming structure 3100 and preventing it from crumpling, folding, sagging or in any other way changing its shape before the seal-forming structure 3100 is affixed to the patient's face.

In one form, the body 3810 may be formed as, or comprise, a frame, for example a skeleton-like structure to provide shape-retaining support to the seal-forming structure 3100 when used.

### 4.5.2 Recess

In exemplary forms, for example the illustrated forms, the body 3810 of applicator 3800 includes a recess 3802. The recess 3802 is formed on one side of the body 3810. The recess 3802 is configured to receive and retain the patient interface 3000 while the patient interface 3000 is put into a therapeutically effective position on the patient's face. In the exemplary form shown in FIG. 13A, the recess 3802 is configured to accommodate the plenum chamber 3200 of the patient interface 3000 with the seal-forming structure 3100 positioned out of the recess 3802. The recess 3802 is configured to receive the patient interface 3000 in such a way that the adhesive surface 3102 of the seal-forming structure 3100 faces away from the body 3810 of the applicator 3800. In certain forms, the recess 3802 may also at least partly accommodate the seal-forming structure 3100 when the patient interface 3000 is received and retained.

It will be appreciated that the recess 3802 may be shaped to match the shape of the component(s) that it is intended to receive and retain but in the negative, i.e. to have a recess where the component(s) has a protrusion, and vice versa.

In the form of the technology shown in FIG. 13A, the body 3810 of applicator 3800 includes two prongs 3806 extending out of the recess 3802. The prongs 3806 are arranged with a size and spacing such that they are able to be inserted into the nares of the patient 1000 while the patient interface 3000 is put into the therapeutically effective position on the patient's face. This helps the patient to correctly position the patient interface 3000 on their face.

The retention of the patient interface 3000 within the recess 3802 until the seal-forming structure 3100 is adhered to the patient's face may be achieved through a friction fit, i.e. the recess 3802 may be sized so as to hold the patient interface 3000 in place with a friction fit. The strength of the friction fit should not be stronger than the adhesive bond between the seal-forming structure 3100 and the patient's face. In this way the applicator 3800, as shown in FIGS. 13C and 14C, may be detached from the patient interface 3000 after the seal-forming structure adheres to the patient's face and the applicator 3800 has been pulled away. Alternatively, the patient interface 3000 may sit in the recess 3802 under the influence of gravity with the applicator 3800 held so that the recess 3802 opens upwards.

In another form, the temporary retention between the patient interface 3000 and the applicator 3800 may be achieved through magnetic attraction. For example, as illustrated in the exemplary form shown in FIG. 17, a magnetic member 3812 may be provided within recess 3802, or alternatively to body 3810 proximate recess 3802, in a position where it can magnetically couple with a magnetic member positioned on the patient interface 3000, for example the first magnetic member 3510 of the inlet port 3202 of the plenum chamber 3200. The magnetic coupling between the magnetic members may be broken easily by tipping the applicator 3800 upwards or downwards after the seal-forming structure 3100 adheres to the patient's face.

In certain forms, the applicator 3800 may be removably attached to an anterior surface of the seal-forming structure 3100 and/or the plenum chamber 3200. In certain forms, the anterior surface of the seal-forming structure 3100 and/or the plenum chamber 3200 that is removably attached to the applicator 3800 may be opposite to the adhesive surface 3102. In certain forms, the applicator 3800 may be configured to adhere to the anterior surface of the seal-forming structure 3100 and/or the plenum chamber 3200. For example, the applicator 3800 may be adhered to the anterior surface of the seal-forming structure 3100 and/or the plenum chamber 3200 through a weakly bonded adhesive such that the applicator 3800 is readily removable by the patient once the patient interface 3000 is in position. The adhesive may be provided on the surface 3808 of the applicator 3800 so that, when the applicator 3800 is removed, little or no adhesive can be felt on the anterior surface of the patient interface 3000. This may help maintain the patient interface 3000 still on the applicator 3800 to make it easier for the patient to fit the interface in the desired position.

In an alternative form, the applicator 3800 may be partially attached removably to the adhesive surface 3102 of the seal-forming structure 3100.

### 4.5.3 Surface

In certain forms, the body 3810 of the applicator 3800 further comprises a surface 3808. Surface 3808 is provided around the recess 3802 to support the seal-forming structure 3100 while the patient interface 3000 is placed into the therapeutically effective position on the patient's face. In certain forms, surface 3808 may form one side or end of the body 3810 of applicator 3800. The width of the body 3810 of the applicator 3800 may be substantially similar to the width and/or length of surface 3808. Alternatively, surface 3808 may be the surface of a flange provided at one end of body 3810.

The surface 3808 may be shaped to complement a region of the patient's face with which the patient interface is configured to form a seal. For example, the surface 3808 may be shaped to match or resemble the region of the patient's face. The surface 3808 may be custom-shaped to complement an individual patient's face shape. Alternatively, the surface 3808 may be generically face shaped. Alternatively, a plurality of applicators 3800 may be provided to suit different face shapes and sizes and each individual patient may select the applicator 3800 having a surface 3808 that most closely resembles their face shape/size.

For example, where the seal-forming structure 3100 is configured to be attached to the alar rim region of the nose, the surface 3808 may be shaped to include a part that complements the alar rim shape of the patient 1000 or a generic alar rim shape. The surface 3808 may include complementary shapes to the corners and creases of the alar rim region such that the profile of the surface 3808 causes the seal-forming structure 3100 to engage closely with the alar rim region when the applicator 3800 is positioned towards the patient's face with the seal-forming structure in the desired position. This causes the seal-forming structure 3100 to adhere to the patient's face and, once the seal-forming structure adheres firmly to the alar rim region, the applicator 3800 can be withdrawn. The surface 3808 may comprise surface features that are shaped to complement other parts of the patient's face to which the seal-forming structure 3100 is configured to adhere, including for example a crease 3232 to assist in adhering the seal-forming structure 3100 in the alar crease, as described earlier in relation to the form of the technology shown in FIGS. 15B and 15C.

Facial features, especially around the nasal ala, alar rim, nasal creases and the naso-labial sulcus include creases and corners whose concavity may be more than the convexity of a finger-tip. As a result, it may be difficult for a patient 1000 to adhere the adhesive surface of the seal-forming structure 3100 to conform closely with the creased regions of the face. In certain forms, the applicator 3800 may be configured to enable accurate locating of the seal-forming structure 3100 and to push the seal-forming structure 3100 into contact with the patient's face in a manner that allows it to follow facial features much more closely than human fingers can achieve. Moreover, the applicator 3800 helps the patient to avoid contacting the adhesive surface of the seal-forming structure 3100 when positioning the patient interface 3000, which can help avoid contamination of the adhesive surface with particulates that can cause loss of adhesive effectiveness. This may occur when the patient 1000 handles the seal-forming structure with their fingers.

In the illustrated exemplary forms of the technology, the surface 3808 entirely surrounds recess 3802. In alternative forms of the technology the surface 3808 may extend outwardly from the recess 3802 on only certain sides of the recess 3802. In certain forms, the body 3810 may comprise a plurality of surfaces 3808 each extending outwardly from the recess 3802 on different sides.

In certain forms the applicator 3800 may comprise one or more guides 3814 for positioning the patient interface 3000 in the desired position in respect of applicator 3800. More particularly, the one or more guides 3814 may be positioned around the periphery of the surface 3808 in order to contain the perimeter of seal-forming structure 3100.

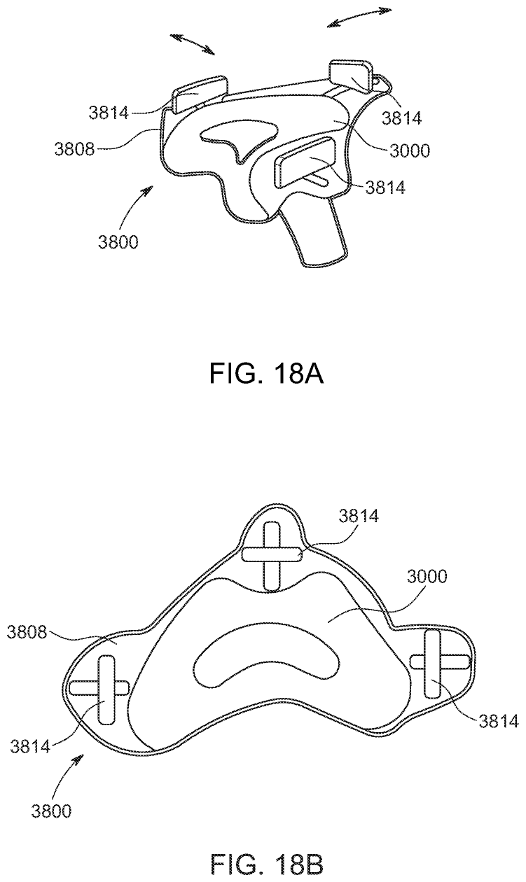

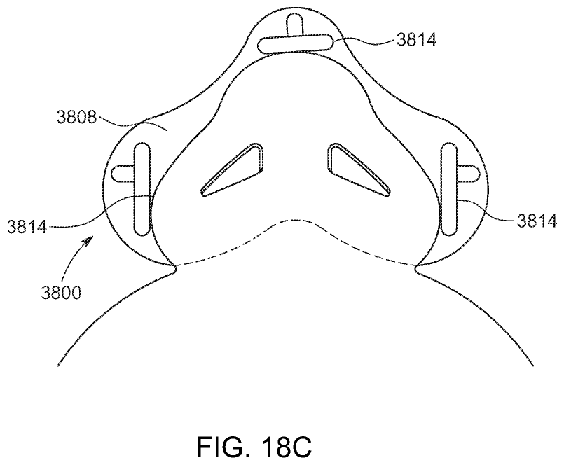

In the exemplary form of the technology shown in FIG. 17, the applicator 3800 comprises a guide 3814 in the form of a ridge extending upwardly from surface 3808 (i.e. in a direction generally perpendicular to the general plane of surface 3808) around a part of the perimeter of surface 3808. In the alternative exemplary form of the technology shown in FIGS. 18A to 18C, the applicator 3800 comprises a plurality of guides 3814. Each guide 3814 comprises a tab extending generally perpendicularly outward from surface 3808. The guides 3814 are arranged in a spaced arrangement around the periphery of surface 3808. In the illustrated form, the tabs are provided on slots and are each able to move along their respective slot to change the size of the area bounded by the tabs within which the seal-forming structure 3100 is to be retained. In use, a patient may alter the position of the tabs in order to most effectively hold the seal-forming structure 3100 of the patient interface 3000 being used in place. The tabs may be held in position through a friction fit within their respective slots.

### 4.5.4 Gripping Portion

In certain forms of the technology, the applicator 3800 further includes a gripping portion 3804 which is configured to be held by the patient 1000 or another user while the patient interface 3000 is put into the therapeutically effective position on the patient's face. The gripping portion 3804 may be provided to the body 3810. In some forms, the gripping portion 3804 and the body 3810 are separate components that are connected together. Alternatively, the gripping portion 3804 and the body 3810 may be integrally formed together, for example molded in one-piece as a single component. The gripping portion 3804 may be provided to the body 3810 on an opposite side to the side of the body 3810 in which the recess 3802 is formed.

In the form of the technology shown in FIGS. 13A, 13B and 13C, the gripping portion 3804 is a handle of length suited to being grasped by a human hand. In the embodiment of FIGS. 14A, 14B and 14C, the gripping portion 3804 is smaller in size and comprises a protrusion on the side of the body 3810 opposite to the recess 3802 that is suitable for being grasped in the fingers of the patient when positioning the patient interface 3000 for use. The gripping portion 3804 may comprise a high friction surface finish.

### 4.6 Manufacturing and/or Shaping the Patient Interface

Forms of the technology provide methods of manufacturing and/or shaping a patient interface 3000 according to the forms of the technology using different methods.

### 4.6.1 Patient Interface Manufacturing Methods

Exemplary methods of manufacturing patient interfaces 3000 according to forms of the technology described elsewhere in this specification will now be described with reference to FIGS. 21A to 21D, 23A, 23B, 24A, 24B, and 25A to 25E.

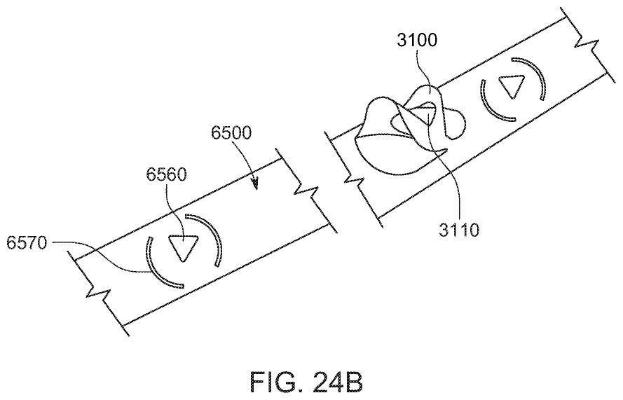

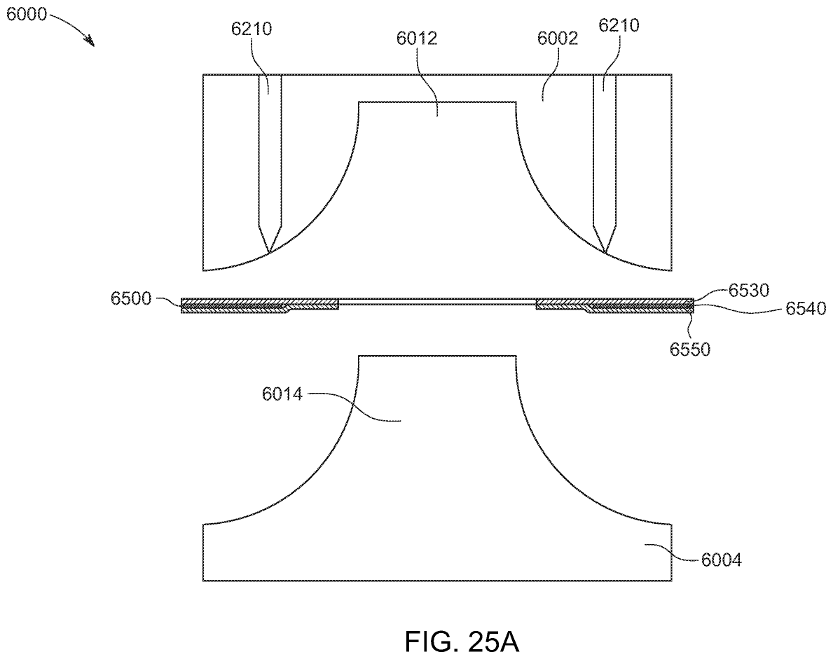

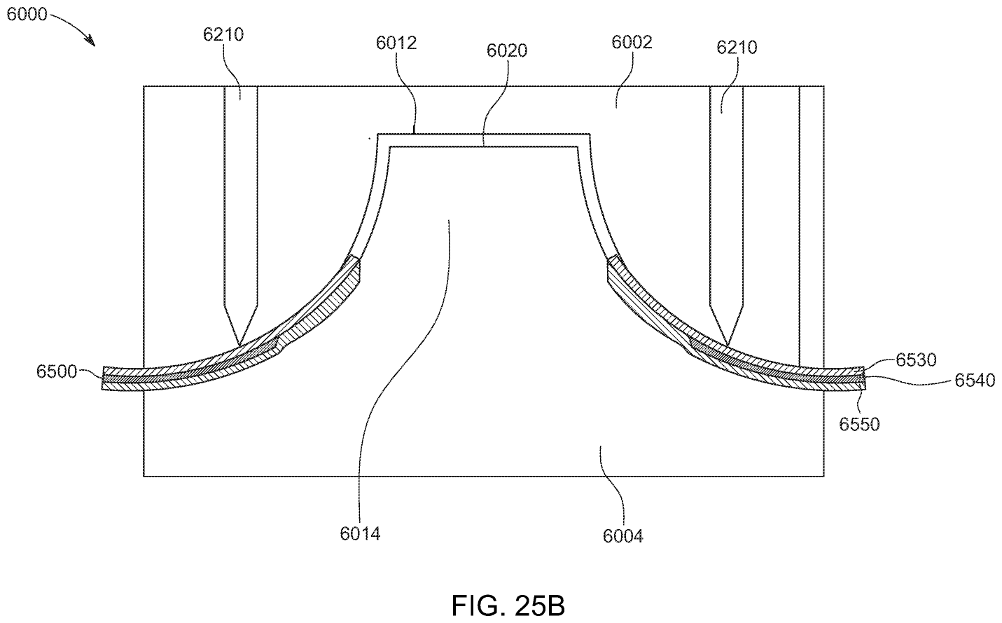

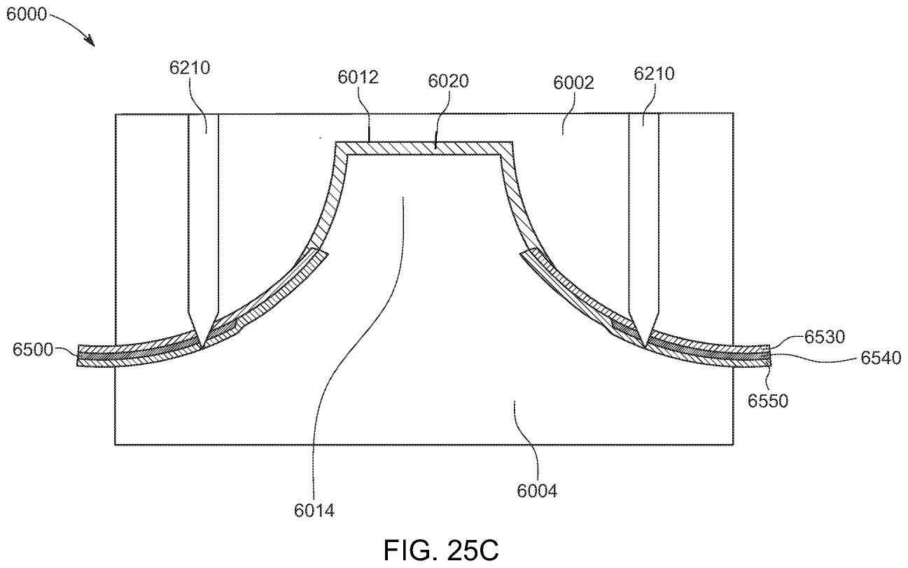

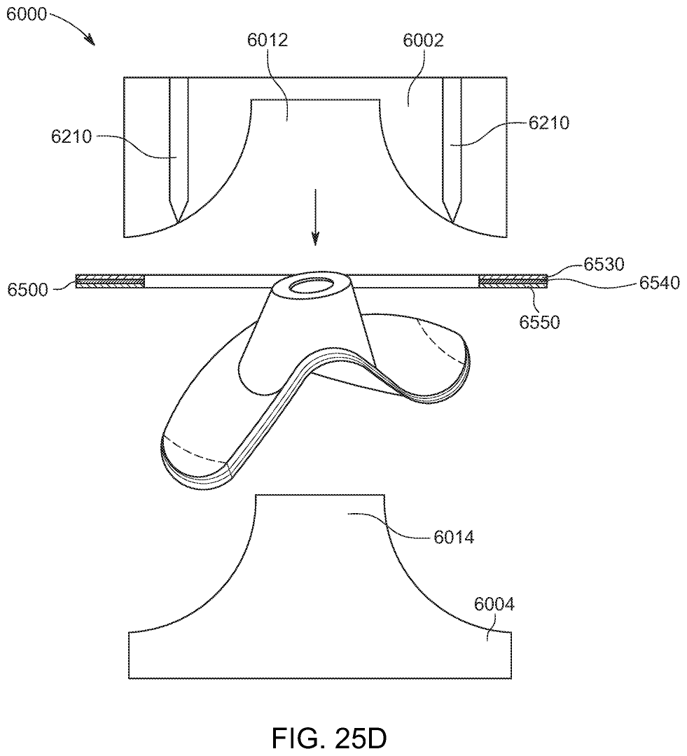

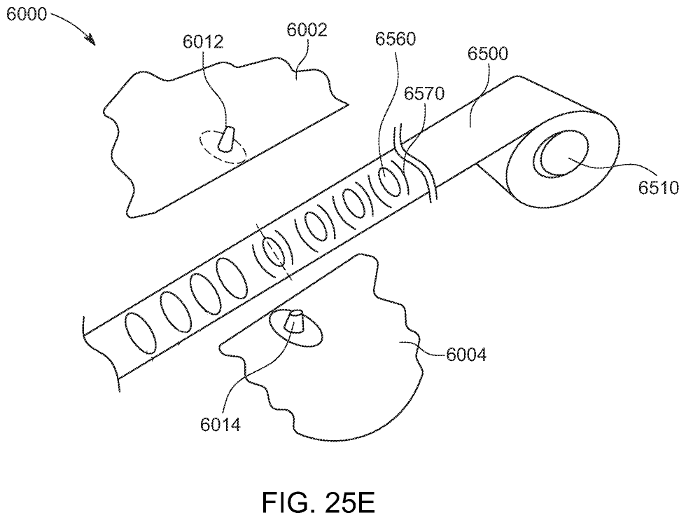

In FIG. 21A, a sheet of material, for example in the form of a tape 6500, from which are formed a plurality of seal-forming structures 3100 is provided. Tape 6500 may be a sheet of adhesive tape or it may be a laminate of multiple layers, including an adhesive layer 6530. Further examples of tape 6500 are illustrated in FIGS. 24A and 25A.

The adhesive layer 6530 may be formed from a backing to which an adhesive is applied. In certain forms, the backing may be formed from polyurethane. This may be useful in forms of the technology using a stretch-release adhesive since polyurethane is pliable and stretchy.

In exemplary forms of the technology, the tape 6500 may be formed as a strip with non-adhesive strips 6540 on one or both sides of the tape 6500. When a seal-forming structure 3100 is cut out of the strip, the cuts may be positioned to form the majority of the seal-forming structure 3100 out of the central section of the tape 6500 comprising an adhesive layer 6530 and to form peripheral sections of the seal-forming structure 3100 from the non-adhesive strips 6540. This process may be used to form a seal-forming structure 3100 with non-adhesive tabs 3108 (formed from the non-adhesive strips 6540) on one or both sides such as described earlier. The non-adhesive strips 6540 may be attached to the adhesive layer 6530 or formed integrally with the adhesive layer 6530 and be regions where no adhesive has been applied.

The tabs 3108, as mentioned earlier, may allow the user to grip the seal-forming structure 3100 to facilitate easy application and/or removal of the seal-forming structure 3100 in use. In addition, if an adhesive (such as an acrylate-based adhesive) is a stretch-release adhesive, the tabs 3108 may be used to grip the seal-forming structure 3100 and pull to stretch it and thereby remove the patient interface from the patient.

In other forms, the non-adhesive strips 6540 may not be present.

The tape 6500 may also comprise a removable layer (liner) 6550, as shown in FIG. 24A and FIG. 25A. The removable layer 6550 covers the adhesive surface of the adhesive layer 6530 to avoid it adhering to anything before use, and to mitigate against loss of adhesion. In addition, the removable layer 6550 may add sufficient strength to the tape 6500 to enable the tape 6500 to be spooled or otherwise manipulated in the manufacturing process with less chance of damaging the tape 6500 than would otherwise be the case.

In the form of technology illustrated in FIG. 25A, the non-adhesive strips 6540 are sandwiched between the adhesive layer 6530 and the removable layer 6550. Once the removable layer 3104 is removed by the patient, the non-adhesive strips 6540 and the adhesive surface 3102 are exposed. The exposed side of the non-adhesive strips 6540 form the tabs 3108 together with the non-adhesive side of the peripheral regions of the adhesive layer 6530 which allow the patient 1000 to grip the patient interface 3000 without touching the adhesive surface 3100.

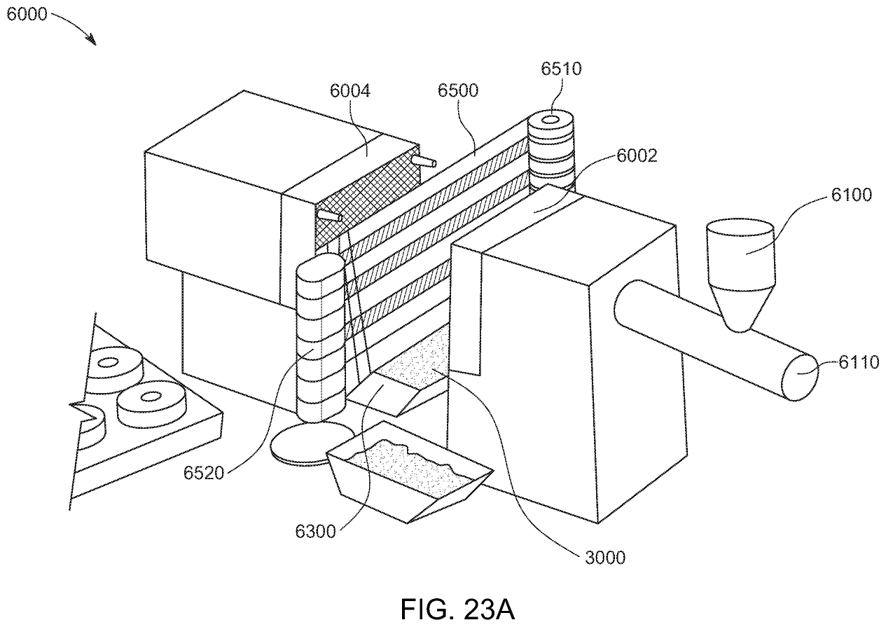

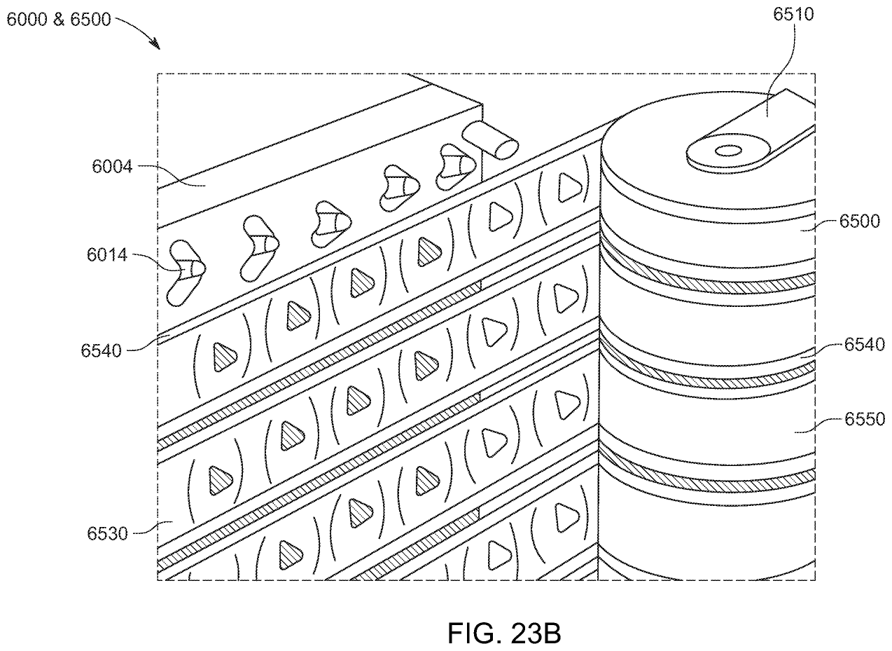

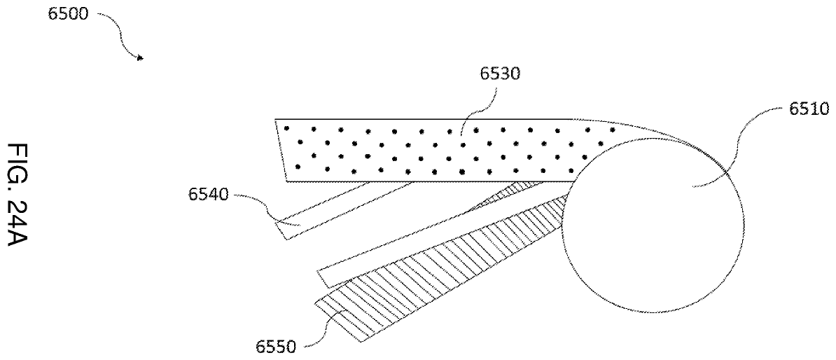

In certain forms, the tape 6500 may be wound onto a first spool 6510. During the manufacturing process, the tape 6500 may wind onto a second spool 6520 as shown in FIG. 23A.

### 4.6.1.1 Cutting

Blanks 3158 for seal-forming structures 3100 may be cut from the sheet of material, for example tape 6500. In exemplary forms, the blanks 3158 may be cut in a form suitable to be shaped into any of the forms of seal-forming structures 3100 described earlier, for example as illustrated by lines indicating the shapes of seal-forming structures in FIG. 21A. In certain forms, the blanks 3158 may be die-cut or laser cut.

In certain forms of the technology, an initial cutting step may involve a partial cut, which may alternatively be referred to as a pre-cut. In the pre-cut step, the blank 3158 is not entirely cut out of the tape 6500. Instead, some parts of the blank 3158 are left connected to the tape 6500. The result of a pre-cut step is shown in FIGS. 23B and 24B according to exemplary forms of the technology. In these figures, tape 6500 is cut to form blanks 3158 still connected to the rest of tape 6500. A pre-cut hole 6560 may be entirely cut out of the middle of each blank 3158. Pre-cut edges 6570 may be cut around the outside of the blanks 3158 in one or more arcs that do not meet up at the ends to leave the blank 3158 still attached to the tape 6500. Alternatively, the tape 6500 may be indented or otherwise marked prior to a cutting step, an advantage of which is explained below.

In certain exemplary forms, as will now be described, the remainder of the cutting of the blanks 3158 from the tape 6500 may occur in a later manufacturing step, for example simultaneously with, or after, a molding process for applying a plenum chamber 3200 to the seal-forming structure blank.

### 4.6.1.2 Molding

The cut-out, or partially cut-out blank 3158 may next be molded into the desired shape for the seal-forming structure 3100.

FIG. 21B illustrates blank 3158 being positioned in between two parts of a molding device 6000. The two parts may be a cavity component 6002, which includes a cavity 6012 into which the blank 3158 may be inserted during the moulding process, and a core component 6004, which includes a protrusion 6014 configured to be inserted into the cavity 6012 in the cavity component 6002 with the blank 3158 positioned in between the cavity and core components. A similar arrangement is illustrated in FIG. 25A.

In the form of the technology illustrated in FIGS. 23A and 23B, the partially cut-out blank is positioned relative to cavity component 6002 and core component 6004 by virtue of spools 6510 and 6520. For example, the spools are arranged to feed the tape 6500 between the cavity component 6002 and core component 6004, which are arranged facing each other with a gap in between. The cavity component 6002 may include multiple cavities 6012, and the core component 6004 may correspondingly include multiple protrusions 6014. Each of the multiple protrusions 6014 are received by a cavity 6014 of the cavity component 6002. The spools may be controlled so that they stop spooling so that the pre-cut blanks 3158 are positioned in line with cavities 6012 and protrusions 6014 on the cavity component 6002 and core component 6004 respectively. Another advantage to pre-cutting (or alternatively pre-indenting or pre-marking) the tape 6500 is that the cuts, indents and/or marks allow a digital sensor to detect the location of the tape 6500 as it moves between the core component 6004 and the cavity component 6002. The digital sensor may be configured to provide data to a processor which controls the first spool 6510 and/or the second spool 6520 to run and/or stop. This allows the tape 6500 to be positioned in such a manner that the holes 6560 are aligned with the protrusions 6014 and cavities 6012 when the core component 6004 is received by the cavity component 6002.

Next the cavity and core components are brought together. This is illustrated in FIGS. 21C and 25B. This step sandwiches the blank 3158 between the cavity and core components, moulding it into the desired shape as dictated by the shape of the cavity 6012 and protrusion 6014. In some forms, a heat forming step may be used in which heat is applied to the blank 3158 to assist with the molding process in some forms. For example, the cavity and core components may conduct heat and there may be heating elements positioned in thermal contact with them. In other forms, no heat forming is applied. It may be desirable not to use heat forming if the adhesive is affected by the application of heat, for example.

In some forms, for example as shown in FIG. 21C, some peripheral parts of the blank 3158 may project outwardly from between the cavity and core components, for example peripheral parts including the tabs 3108.

In some forms, the molding device may comprise, or have provided to it, a cutter. The cutter may comprise cutting blades 6210 having cutting ends 6200. The cutting blades 6210 may be provided on either side of each cavity 6012 on the cavity component 6002. In another form, the cutting ends 6200 may be configured to surround the cavity component 6002, completely or partially. In another form, the cutting ends may be arranged to align with those parts of the blanks 3158 that remain joined to the tape 6500. The cutting blades 6210 may alternatively be provided to the core component 6004 instead. Such a form is illustrated in FIGS. 25A to 25E. In this form, the cutting blades 6210 may be positioned inside channels in the cavity component 6002 (or core component 6004) and be configured to move, longitudinally, inside the channels towards the core component 6004 (or cavity component 6002).

In use, as shown in FIG. 25C, the cutting ends 6200 may be made to move towards the blank 3158, thereby cutting the blank 3158 entirely out of the tape 6500. This may occur before, simultaneously with, or after the overmolding process that will now be described. Alternatively, or additionally, a similar cutting step may trim excessive tap material from the blank 3158 in order to form the seal-forming structure 3100.

In certain forms of the technology, while the blank 3158 for the seal-forming structure 3100 is being held in the molding device, the plenum chamber 3200 is provided to the seal-forming structure 3100 by an overmolding process. Such a process step is illustrated in FIGS. 21D and 25C. The cavity component 6002 may comprise a channel 3908 through which a molten overmold material, for example TPE or silicon may be injected into contact with the blank 3158. The molten overmold material may be injected from channel 3908 into a gap between the cavity 6012 and the blank 3158. This gap may be shaped in the desired shape for the plenum chamber 3200 so that the plenum chamber 3200 is formed as an overmold on the seal-forming structure 3100.

As illustrated in FIG. 23A, the molding device 6000 may include a hopper 6100 which is a reservoir of the molten overmold material. The molten overmold material is made to flow into the gap 6020 through a conduit 6110 and channel 3908.

When the cavity component 6002 and core component 6004 are pulled apart, the shaped, overmolded plenum chamber 3200 and seal-forming structure 3100 is formed and the molding device 6000 may be formed so that this component falls into a collector 6300.

In certain forms of the technology, shape retainers 3140 may be overmolded onto blank 3158 in a similar fashion to that described above for the plenum chamber 3200, with the cavity component 6002 forming one or more gaps with the blank 3158 suitable for receiving the overmold material in order to form the shape retainers 3140.

### 4.6.2 Patient Interface Customisation

In one form, a patient interface 3000 is customised to fit an individual patient 1000. In another form, a patient interface 3000 is manufactured which is generically shaped and designed to fit a general face shape. In a still further form, a patient interface 3000 is manufactured in a limited plurality of different shapes and sizes so that there is a limited plurality of pre-formed patient interfaces 3152 and any given patient 1000 selects the pre-formed patient interface 3150 out of the plurality of pre-formed patient interfaces 3152 that best suits their needs.

As mentioned before, manufacturing, supplying and distributing generically shaped and sized pre-formed patient interfaces 3150 on a large scale may be less expensive than custom-making seal-forming structures 3100 to correspond to the exact facial features of each individual patient 1000. However, seal-forming structures 3100 that imitate facial features have a number of advantages, as mentioned earlier. Shaping pre-formed patient interfaces 3150 in a plurality of different shapes and sizes may provide a useful compromise to leverage some of the advantages of economies of scale while still providing seal-forming structures 3100 that may fit an individual patient to a sufficient degree. The greater the plurality of shapes and sizes provided, the greater the prospect of a patient being able to select a patient interface that closely fits them. The fewer the plurality of shapes and sizes, the greater the economies of scale.

Methods of customizing a patient interface 3000 for use in delivering breathable gas to a patient according to exemplary forms of the technology will now be described in further detail.

### 4.6.2.1 Receiving Patient Data

In one exemplary form, a method of customizing a patient interface 3000 includes a first step of receiving data indicative of shape and size of a region of a patient's face surrounding an entrance to the patient's airways. The method may further include a step of scanning the region of the patient's face to generate the data indicative of the shape and size of the region of the patient's face.

### 4.6.2.2 Selecting a Pre-Formed Patient Interface Assembly

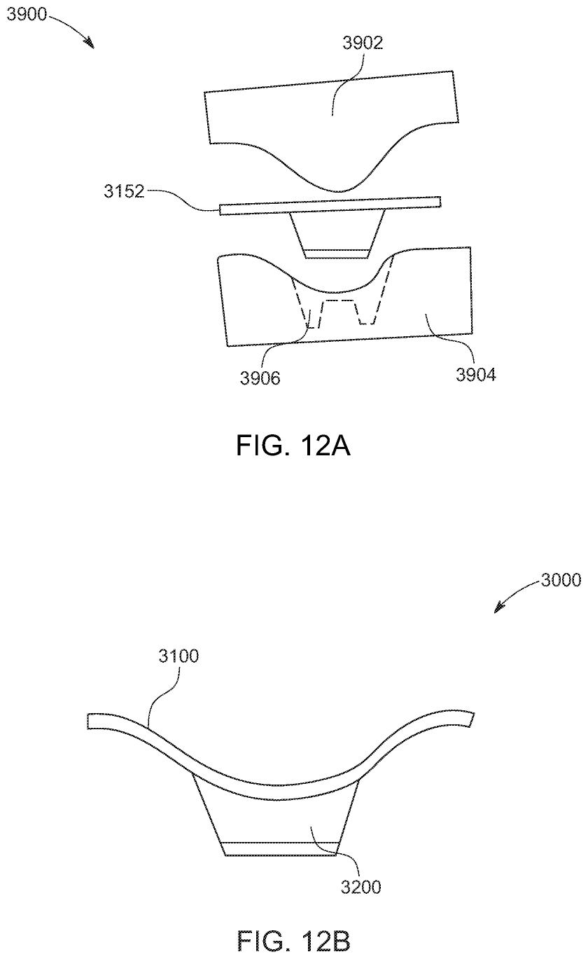

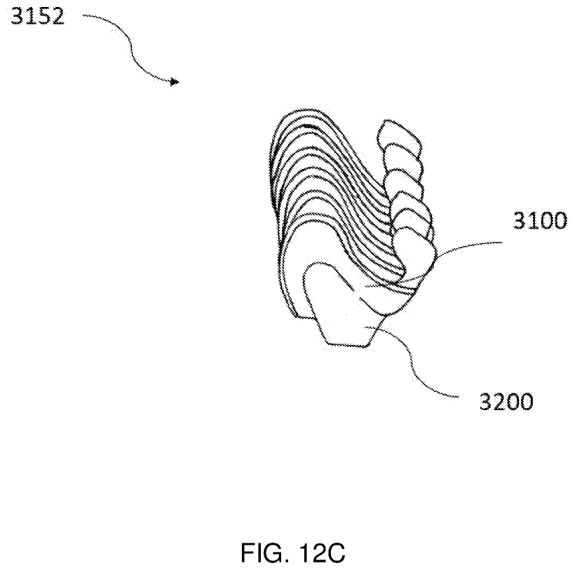

Further, the method may include a second step of selecting a pre-formed patient interface 3150 from a plurality of pre-formed patient interfaces 3152, as shown in FIG. 12C. Each pre-formed patient interface 3150/3152 comprises a plenum chamber 3200 and a seal-forming structure 3100 according to any of the forms of technology described elsewhere in this document.

In one form of the technology, each of the plurality of pre-formed patient interfaces 3152 may have a different shape and/or size. For example, the plurality of pre-formed patient interfaces 3152 may be a series in which each pre-formed patient interface 3150 varies in dimension from the next pre-formed patient interface 3150 in the series. The plurality of pre-formed patient interfaces 3152 may also vary in shape according to typical facial types in the population. For example, there may be pre-formed patient interfaces 3152 corresponding to different sexes, ages, ethnicities, nose types, etc.

A pre-formed patient interface 3150 which most closely conforms to the region of the patient's face with which the seal-forming structure 3100 is to adhere is chosen from the plurality of pre-formed patient interfaces 3152.

In an alternative form of the technology, each of the plurality of pre-formed patient interfaces 3152 is identical or there are a very small number of different pre-formed patient interfaces 3152. This reduces the cost of manufacturing the pre-formed patient interfaces 3152 (for example the cost of making molds) but means that it may be more difficult to find a closely confirming pre-formed patient interface for some patients.

### 4.6.2.3 Molding

In a subsequent step in exemplary forms, the selected pre-formed patient interface 3152, for example the seal-forming structure thereof, is shaped to more closely match/resemble the facial region of the individual patient 1000, the facial regions being those to which the seal-forming structure 3100 when shaped from the pre-formed patient interface 3150 is to adhere.

In one exemplary form, the step of shaping the selected pre-formed patient interface 3152 comprises molding the pre-formed patient interface 3152 to the desired shape.

For example, in the form of the technology shown in FIGS. 12A and 12B, a two-part molding device 3900 is used to shape the selected pre-formed patient interface 3152. The molding device 3900 includes a first part 3902 and a second part 3904. The selected pre-formed patient interface 3152, as shown in FIG. 12A, is placed between the first part 3902 and the second part 3904. The two parts 3902, 3904 of the molding device 3900 are pressed together to shape the selected pre-formed patient interface 3152. More particularly, the molding device 3900 may be used to shape the seal-forming structure 3100 of the selected pre-formed patient interface 3152. This may be the most important part to shape since the seal-forming structure 3100 is the part of the patient interface 3000 that contacts the patient's face in use. In one form, as shown in FIGS. 12A and 12B, the second part 3904 of the molding device 3900 includes a recess 3906 to receive the plenum chamber 3200 so that the plenum chamber 3200 remains unchanged during the molding process. The plenum chamber 3200 of the selected pre-formed patient interface 3152 may additionally, or alternatively be shaped by the molding device 3900 in some forms of the technology.

The first part 3902 and the second part 3904 of the molding device 3900 may be shaped to produce a seal-forming structure 3100 which is substantially close to the facial regions to which the seal-forming structure 3100 is attached to. In order to achieve this, the first part 3902 and/or the second part 3904 are shaped according to the facial regions using the data indicative of the shape and size of the region of the patient's face.

In one form, the concerned facial regions of the patient 1000 are 3-D image scanned and the first part 3902 and the second part 3904 may be formed in accordance to the scanned virtual images. The first part 3902 and the second part 3904 may be 3-D printed.

In certain forms, the molding device 3900 may be a thermo-forming device which thermo-forms the selected pre-formed patient interface 3152, or a part thereof, for example the seal-forming structure 3100, into the desired shape.

In alternative forms, the molding device 3900 may be an injection molding device which shapes the pre-formed patient interface 3512 and retains its shape by pouring an injection molding substrate over the shaped pre-formed patient interface 3152. In some forms, the substrate is overmolded over the shaped pre-formed patient interface 3152.

In one form of the technology, the pre-formed patient interface 3152 may be formed by cutting, shaping and optionally overmolding the blanks 3158 in the manner explained above, and in relation to FIGS. 21A to 21D or 23A to 25E, for example.

### 4.6.2.4 Laser Bending

In one form (not shown in the Figs), a selected pre-formed patient interface 3152 may be shaped through laser bending.

### 4.6.2.5 3-D Printing

In one form (not shown in the Figs), a patient interface 3152 may be produced through 3-D printing.

### 4.7 RPT Device

An RPT device 4000 in accordance with one aspect of the present technology comprises mechanical, pneumatic, and/or electrical components and is configured to execute one or more algorithms, such as any of the methods, in whole or in part, described herein. The RPT device 4000 may be configured to generate a flow of air for delivery to a patient's airways, such as to treat one or more of the respiratory conditions described elsewhere in the present document.

In certain forms, the RPT device 4000 may be configured to deliver a flow of air to the patient interface 3000 at a positive pressure with respect to ambient. The RPT device 4000 may be configured to deliver air at a therapeutic pressure, for example at least 6 cmH₂O with respect to ambient. Conventional RPT devices 4000 may be used for this purpose.

In other forms, the RPT device 4000 may be configured to deliver a flow or air to the patient interface 3000 at a lower pressure (but still at a positive pressure relative to ambient), for example 2 to 6 cmH₂O with respect to ambient. Respiratory therapy systems incorporating RPT devices 4000 delivering a flow of air at such pressures may be useful for providing low level therapy. For example, such systems may be useful for treating, or ameliorating snoring, or other mild respiratory conditions. Compared to an RPT device that is able to deliver air at higher pressures, for example RPT devices that may be suitable for treating obstructive sleep apnea, such systems may be cheaper to manufacture, use less power and be more compact in size.

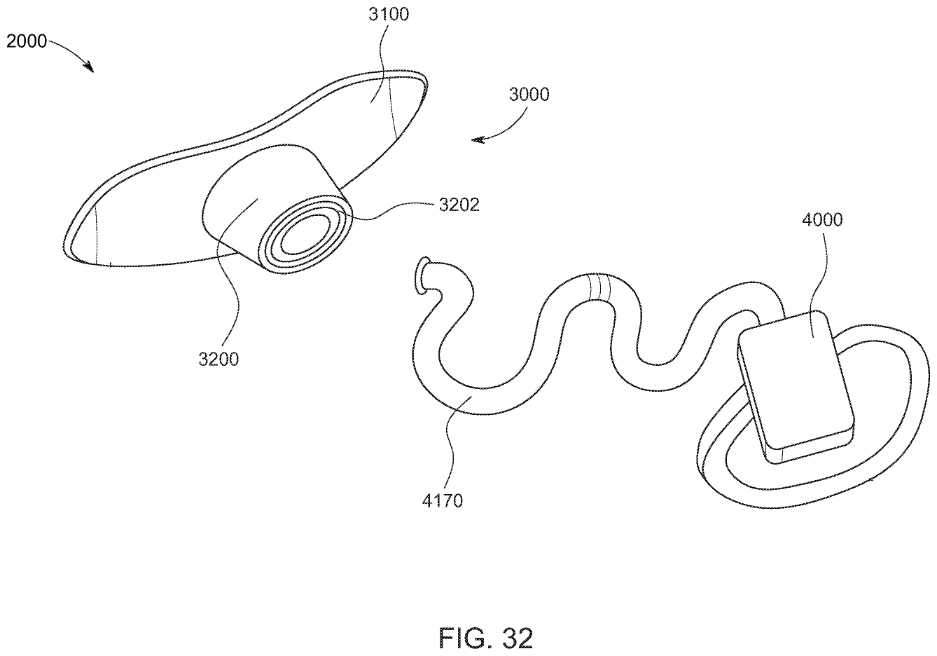

FIG. 32 illustrates a respiratory therapy system 2000 incorporating a RPT device 4000 of the type just described. In the form shown in FIG. 32, breathable gas is conveyed to the patient interface 3000 RPT device 4000 which is compact in size and may therefore be portable, i.e. able to be carried by the patient during use, e.g. mounted on the patient's person or clothing. For example, the RPT device 4000 may be strapped around the patient's neck or arm, or carried in a pocket, in use.

Breathable gas from the RPT device 4000 may be conveyed to the patient interface 3000 through an air circuit 4170. The inlet port 3202 of the plenum chamber 3200 may be connected to an end of the air circuit 4170 with the other end of the air circuit 4170 being connected to the RPT device. Since the flow rate and/or pressure of the supply of air may be lower than with a conventional RPT device 4000 (e.g. a CPAP device), the air circuit 4170 may have a reduced diameter compared to conventional air circuits. For example, in certain forms, the air circuit 4170 may have a diameter in the range 5-15 mm, for example 10 mm. A smaller diameter tube provides more impedance to the flow of air than a larger diameter tube but this is acceptable if the flow rate and/or pressure to be delivered is also low. A smaller diameter tube may be desirable as being less bulky and obtrusive, easier to store or package, and cheaper to manufacture.

### 4.8 Glossary

For the purposes of the present technology disclosure, in certain forms of the present technology, one or more of the following definitions may apply. In other forms of the present technology, alternative definitions may apply.

### 4.8.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB (Sleep Disordered Breathing) events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm2 and hectopascal. 1 cmH₂O is equal to 1 g-f/cm2 and is approximately 0.98 hectopascal (1 hectopascal=100 Pa=100 N/m2=1 millibar˜0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.8.1.1 Materials

Silicone or Silicone Elastomer: A synthetic rubber. In this specification, a reference to silicone is a reference to liquid silicone rubber (LSR) or a compression molded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 4.8.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH₂O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 4.8.2 Anatomy

### 4.8.2.1 Anatomy of the Face

Ala: the external outer wall or “wing” of each nostril (plural: alar)

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius):

Lip, upper (labrale superius):

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture. Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

4.8.2.2 Anatomy of the respiratory system

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air-filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 4.8.3 Patient Interface

Anti-asphyxia valve (AAV): The component or sub-assembly of a mask system that, by opening to atmosphere in a failsafe manner, reduces the risk of excessive CO2 rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Functional dead space: (description to be inserted here)

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.9 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 4.10 REFERENCE SIGNS LIST

1000 Patient
3132 Septum Region
3141 Alar Rim Region
3142 Superior-most Region of Lip Superior
3143 Anterior Region of the Nose Inferior to the Pronasale
3144 Region Inferior to the Alar Crest Point
3145 Regions of the Cheeks Adjacent the Nasal Alar Crest Point
2000 Respiratory Therapy System
2100 Sensor Module
2110 Tube of Sensor Module
2112 Electrodes of Sensor Module
2114 Cap of Sensor Module
3000 Patient Interface
3100 Seal-Forming Structure
3102 Adhesive Surface
3104 Removable Layer
3104T Tab
3105 First Part
3106 Second Part
3105T First Tab
3106T Second Tab
3108 Tabs
3110 Opening of the Seal-Forming Structure
3112 Flange
3114 Inner Surface of the Flange
3116 Outer Surface of the Flange
3120 First Seal-Forming Structure
3130 Second Seal-Forming Structure
3140 Shape Retainer
3150 Pre-Formed Patient Interface
3152 Plurality of Pre-Formed Patient Interfaces
3158 Blank
3160 Notch
3172 Apex Portion of the Seal-Forming Structure
3174 Shoulder Portions of the Seal-Forming Structure
3176 Corner Portions of the Seal-Forming Structure
3178 Stem Portion of the Seal-Forming Structure
3180 Recess of the Seal-Forming Structure
3182 Convex Protrusion of the Seal-Forming Structure
3184 Edge of the Seal-Forming Structure
3186 Obtuse Corners of the Seal-Forming Structure
3188 Concave Recess of the Seal-Forming Structure
3190 Concave Protrusion of the Seal-Forming Structure
3200 Plenum Chamber
3202 Plenum Chamber Inlet Port
3216 Annular Ring
3230 Front Layer
3232 Crease(s)
3250 Nasal Pillows
3252 Frusto-cone
3254 Stalk
3300 Integral/Single Component
3400 Vent
3402 Anti-Asphyxia Valve
3404 Expiratory Resistance Valve
3406 Tube of Expiratory Resistance Valve
3408 Cap of Expiratory Resistance Valve
3409 Valve Components of Expiratory Resistance Valve
3410 Diffuser
3420 Tube
3422 First Vent
3424 Second Vent
3426 First End of Tube
3428 Second End of Tube
3500 Decoupling Structure
3502 Tube
3504 Flexible section
3510 First Magnetic Member
3512 Second Magnetic Member
3514 Adapter
3518 Frame
3520 Prongs of the Air Circuit
3522 Receiver of the Tube
4170 Air Circuit
3600 Connection Port
3700 Heat and Moisture Exchanger
3710 Neck of the Adapter
3712 First flange of the Adapter
3714 Second flange of the Adapter
3800 Applicator
3802 Recess of the Applicator
3804 Gripping Portion
3806 Prongs of the Applicator
3808 Surface of the Applicator
3810 Body of the Applicator
3812 Magnetic Member
3814 Guide(s) of Applicator
3908 Channel
4000 RPT device
5000 Patient Interface System
6000 Molding device
6002 Cavity Component
6004 Core Component
6012 Cavity (or Cavities)
6014 Protrusion
6020 Gap
6100 Hopper
6110 Conduit
6200 Cutting End(s)
6210 Cutting Blade
6300 Collector
6500 Tape
6510 First Spool
6520 Second Spool
6530 Adhesive Layer
6532 First Layer Assembly
6532A Backing Layer of First Layer Assembly
6532B Adhesive Layer of First Layer Assembly
6534 Second Layer Assembly
6534A Backing Layer of Second Layer Assembly
6534B Adhesive Layer of Second Layer Assembly
6540 Non-Adhesive Strips
6550 Removable Layer
6560 Pre-cut hole(s)
6570 Pre-cut edge(s)

1.-46. (canceled)

47. A patient interface for use in delivering breathable gas to a patient, the patient interface comprising:
a plenum chamber pressurisable to a therapeutic pressure of at least 6 cmH₂O above ambient air pressure, said plenum chamber including a plenum chamber inlet port configured to receive a flow of breathable gas at the therapeutic pressure for breathing by the patient;
a seal-forming structure provided to the plenum chamber, wherein the seal-forming structure is configured to form a seal with a region of the patient's face surrounding an entrance to the patient's airways, said seal-forming structure having an opening therein such that the flow of breathable gas is delivered to at least an entrance to the patient's nares, the seal-forming structure being configured to maintain said therapeutic pressure in the plenum chamber throughout the patient's respiratory cycle in use; and
a vent structure to allow a continuous flow of gases exhaled by the patient from an interior of the plenum chamber to ambient, said vent structure being configured to maintain the therapeutic pressure in the plenum chamber in use,
wherein the seal-forming structure comprises at least one adhesive surface configured in use to adhere to a region of the patient's face to form the seal, and
wherein the region of the patient's face surrounds both the patient's nares and the region comprises side regions of the patient's nasal alar and a superior region of the patient's lip superior.

48. A patient interface as claimed in claim 47, wherein the region of the patient's face comprises the patient's pronasale region.

49. A patient interface as claimed in claim 48, wherein the region of the patient's face comprises an anterior region of the nose that is immediately inferior to the patient's pronasale.

50. A patient interface as claimed in claim 47, wherein the region of the patient's face comprises inferiorly facing surfaces of the nasal alar.

51. A patient interface as claimed in claim 47, wherein the seal-forming structure is configured such that, when the seal-forming structure is adhered to the patient's face, a superior-most portion of the seal-forming structure adheres to the patient's alar crease.

52. A patient interface as claimed in claim 47, wherein the region of the patient's face comprises regions of the patient's cheeks adjacent to the nasal alar.

53. A patient interface as claimed in claim 52, wherein the region of the patient's face comprises regions between the patient's nasal alar and the patient's nasolabial sulcus.

54. A patient interface as claimed in claim 47, wherein the seal-forming structure is configured so that the shape of the at least one adhesive surface substantially resembles the region of the patient's face.

55. A patient interface as claimed in claim 47, wherein the seal-forming structure is a substantially D-shaped band.

56. A patient interface as claimed in claim 47, wherein the seal-forming structure comprises a notch in an edge of a region of the seal-forming structure configured to adhere to a region of the patient's nose substantially inferior to the patient's pronasale in use.

57. A patient interface as claimed in claim 47, wherein a first region of the at least one adhesive surface adheres to the patient's face during use with a greater adhesion strength than a second region of the at least one adhesive surface.

58. A patient interface as claimed in claim 47, wherein the seal-forming structure has a thickness in the range of approximately 0.2 mm to 0.3 mm when the patient interface is worn by the patient.

59. A patient interface as claimed in claim 47, wherein the seal-forming structure and the plenum chamber are integrally formed as a single component.

60. A patient interface as claimed in claim 47, wherein the adhesive surface of the seal-forming structure is provided with an adhesive that is suitable for at least one re-application of the seal-forming structure on the patient's face.

61. A patient interface as claimed in claim 47, wherein the seal-forming structure is a first seal-forming structure and the patient interface further comprises a second seal-forming structure, wherein the first and second seal-forming structures are interchangeably connectable to the plenum chamber.

62. A patient interface as claimed in claim 47, wherein the seal-forming structure is formed from silicone or a thermoplastic elastomer.

63. A patient interface as claimed in claim 47, wherein the adhesive surface comprises any adhesives selected from the following group: rubber zinc oxide adhesive; acrylic adhesive; acrylate adhesive; and silicone adhesive.

64. A patient interface for use in delivering breathable gas to a patient, the patient interface comprising:
a plenum chamber pressurisable to a therapeutic pressure of at least 6 cmH₂O above ambient air pressure, said plenum chamber including a plenum chamber inlet port configured to receive a flow of breathable gas at the therapeutic pressure for breathing by the patient;
a seal-forming structure provided to the plenum chamber, wherein the seal-forming structure is configured to form a seal with a region of the patient's face surrounding an entrance to the patient's airways, said seal-forming structure having an opening therein such that the flow of breathable gas is delivered to at least an entrance to the patient's nares, the seal-forming structure being configured to maintain said therapeutic pressure in the plenum chamber throughout the patient's respiratory cycle in use; and
a vent structure to allow a continuous flow of gases exhaled by the patient from an interior of the plenum chamber to ambient, said vent structure being configured to maintain the therapeutic pressure in the plenum chamber in use,
wherein the seal-forming structure comprises at least one adhesive surface configured in use to adhere to a region of the patient's face to form the seal, and
wherein the region of the patient's face surrounds both the patient's nares and the region comprises inferiorly facing surfaces of the patient's nasal alar and a superior region of the patient's lip superior.

65. A patient interface as claimed in claim 64, wherein the region of the patient's face comprises side regions of the nasal alar.

66. A patient interface as claimed in claim 64, wherein the region of the patient's face comprises the patient's pronasale region
